(19) United States
(12) Patent Application Publication
(10) Pub. No.: US 2014/0228349 A1
(43) Pub. Date: Aug. 14, 2014

(54) 5,7-SUBSTITUTED-IMIDAZO[1,2-C]PYRIMIDINES

(71) Applicant: Array BioPharma Inc., Boulder, CO (US)

(72) Inventors: Mark Laurence Boys, Boulder, CO (US); Laurence E. Burgess, Boulder, CO (US); C. Todd Eary, Boulder, CO (US); Robert Groneberg, Boulder, CO (US); Bruno P. Hache, Boulder, CO (US); Darren Harvey, Boulder, CO (US); Erik James Hicken, Boulder, CO (US); Christopher F. Kraser, Boulder, CO (US); Ellen Laird, Boulder, CO (US); David A. Moreno, Boulder, CO (US); Mark C. Munson, Boulder, CO (US); Li Ren, Boulder, CO (US); John E. Robinson, Boulder, CO (US); Stephen T. Schlachter, Boulder, CO (US)

(21) Appl. No.: 14/351,415

(22) PCT Filed: Oct. 9, 2012
(86) PCT No.: PCT/US12/59282

§ 371 Date: Apr. 11, 2014

Related U.S. Application Data
(60) Provisional application No. 61/546,426, filed on Oct. 12, 2011.

(51) Int. Cl. C07D 487/04

(52) U.S. Cl. CPC C07D 487/04
USPC 514/210.18; 544/281; 514/210.21; 514/259.1

(57) ABSTRACT

Compounds of Formula I: and stereoisomers and pharmaceutically acceptable salts and solvates thereof in which R¹, R², R³, R⁴, R⁵, R⁶, X¹ and X² have the meanings given in the specification, are inhibitors of one or more JAK kinases and are useful in the treatment of autoimmune diseases, inflammatory diseases, rejection of transplanted organs, tissues and cells, as well as hematologic disorders and malignancies and their co-morbidities.

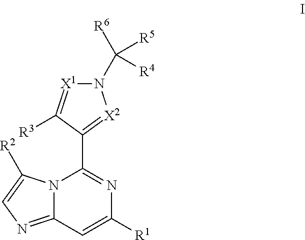

The present invention relates to novel compounds, to pharmaceutical compositions comprising the compounds, to processes for making the compounds, and to the use of the compounds in therapy. More particularly, it relates to certain 5,7-substituted-imidazo[1,2-c]pyrimidine compounds which are inhibitors of JAK kinases. In particular, the compounds are inhibitors of Tyk2, JAK1, JAK2, and/or JAK3, and are useful in the treatment of JAK kinase-associated diseases such as autoimmune diseases, inflammatory diseases, organ, tissue and cell transplant rejection, and hematological disorders and malignancies.

The members of the Janus kinase (JAK) family of non-receptor, intracellular tyrosine kinases are components of cytokine signal transduction. Four family members have been identified: JAK1, JAK2, JAK3 and Tyk2. The JAKs play a key role in the intracellular signaling mediated through Type I and Type II cytokine receptors. Specific cytokine receptor chains are associated with particular JAK kinases (reviewed in O'Sullivan et al., Mol. Immunol., 2007, 44:2497; Murray J., Immunol., 2007, 178:2623). Upon binding of cytokines to their receptors, JAKs are activated and phosphorylate the receptors, creating docking sites for other signaling molecules, in particular members of the signal transducer and activator of transcription (STAT) family. Upon phosphorylation, STATs dimerize, translocate to the nucleus and activate expression of genes involved in development, growth, differentiation, and maintenance of a variety of cell types. The cytokine-induced responses mediated by JAK kinases are important in host defense and, when dysregulated, play a role in pathogenesis of immune or inflammatory diseases, immune deficiencies, and malignancy (O'Sullivan et al., Mol. Immunol. 2007, 44:2497). Elevated or decreased levels of JAK/STAT-utilizing cytokines have been implicated in a number of disease states. In addition, mutations or polymorphisms in Type 1 and II cytokine receptors, JAK kinases, STAT proteins, and JAK/STAT regulatory proteins such as phosphotyrosine phosphatases, SOCS proteins, PIAS proteins have been reported in a variety of diseases. When dysregulated, JAK-mediated responses can positively or negatively effect cells leading to over-activation and malignancy or immune and hematopoietic deficiencies, respectively, and suggests the utility for use of inhibitors of JAK kinases. The JAK/STAT signaling pathway is involved in a variety of hyperproliferative and cancer-related processes including cell-cycle progression, apoptosis, angiogenesis, invasion, metastasis and evasion of the immune system (Haura et al., Nature Clinical Practice Oncology, 2005, 2(6), 315-324; Verna et al., Cancer and Metastasis Reviews, 2003, 22, 423-434). In addition, the JAK/STAT signaling pathway is important in the genesis and differentiation of hematopoietic cells and regulating both pro- and antiinflammatory and immune responses (O'Sullivan et al., Molecular Immunology 2007, 44:2497. Because cytokines utilize different patterns of JAK kinases (O'Sullivan et al., Mol. Immunol., 2007, 44:2497; Murray J., Immunol., 2007, 178:2623), there may be utility for antagonists of JAK kinases with differing intra-family selectivity profiles in diseases associated with particular cytokines or in diseases associated with mutations or polymorphisms in the JAK/STAT pathways.

JAK3 deficient mice exhibit a severe combined immunodeficiency syndrome (scid). The failure of lymphocyte development in an otherwise healthy animal supports the utility of targeting JAK3 for diseases associated with lymphocyte activation.

In addition to the scid phenotype of the JAK3-deficient mice, the elevated expression of cytokines which signal through the JAK3-associated gamma common chain in inflammatory and immune responses suggests that inhibitors of JAK3 could impede T-cell activation and prevent rejection of grafts following transplant surgery, or to provide therapeutic benefit to patients suffering autoimmune or inflammatory disorders (reviewed in O'Sullivan et al., Mol. Immunol., 2007, 44:2497; Murray J., Immunol., 2007, 178:2623).

Inhibitors of the tyrosine kinase JAK3 have been described to be useful as immunosuppressants (see, for example, U.S. Pat. No. 6,313,129; Borie et al., Curr. Opin. Investigational Drugs, 2003, 4:1297). JAK3 has also been shown to play a role in mast-cell mediated allergic reactions and inflammatory diseases.

JAK1- and JAK2-deficient animals are not viable. Studies have identified a high prevalence of an acquired activating JAK2 mutation (JAK2V617F) in myeloproliferative disorders such as polycythemia vera, essential thrombocythemia and idiopathic myelofibrosis and to a lesser extent in several other diseases. The mutant JAK2 protein is able to activate downstream signaling in the absence of cytokine stimulation, resulting in autonomous growth and/or hypersensitivity to cytokines and is believed to play a role in driving these diseases (Percy, M. J. and McMullin M. F., Hematological Oncology, 2005, 23(3-4), 91-93). Additional mutations or translocations resulting dysregulated JAK2 function have been described in other malignancies (Ihle J. N. and Gilliland D. G., Curr. Opin. Genet. Dev., 2007, 17:8; Sayyah J. and Sayeski P. P., Curr. Oncol. Rep., 2009, 11:117). Inhibitors of JAK2 have been described to be useful in myeloproliferative diseases (Santos et al., Blood, 2010, 115:1131; Barosi G. and Rosti V., Curr. Opin. Hematol., 2009, 16:129, Atallah E. and Versotvsek S., 2009 Exp. Rev. Anticancer Ther. 9:663). More rarely, mutations in JAK1 and JAK3 have been reported in hematologic malignancies (Vainchecker et al., Semin. Cell Dev. Biol., 2008, August 1; 9(4):385-93). JAK family kinase inhibitors may be useful in these settings (Sayyah J. and Sayeski P. P., Curr. Oncol. Rep., 2009, 11:117). In addition, over expression of cytokines which utilize JAK2 for signaling have been implicated in disease states (JAK2 utilizing cytokines are reviewed in O'Sullivan et al., Mol. Immunol., 2007, 44:2497; Murray J., Immunol., 2007, 178:2623).

JAK1 has been reported to signal with other JAK1 molecules or in collaboration with JAK2 or JAK3 depending on the cytokine input (JAK1 utilizing cytokines reviewed in O'Sullivan 2007, Murray 2007). Elevated levels of cytokines which signal through JAK1 have been implicated in a number of immune and inflammatory diseases. JAK1 or JAK family kinase antagonists may be useful for modulating or treating in such diseases.

Tyk2-deficient animals exhibit blunted immune responses to several types of pathogens and are less susceptible to some autoimmune diseases. This phenotype supports the utility of inhibiting Tyk2 in particular disease settings. Particularly, targeting Tyk2 appears to be a promising strategy for the treatment of IL-12-, IL-23- or Type 1 IFN-mediated diseases or diseases. These include but are not limited to rheumatoid arthritis, multiple sclerosis, lupus, psoriasis, psoriatic arthritis, inflammatory bowel disease, uveitis, and sarcoidosis (Shaw, M. et al., Proc. Natl. Acad. Sci. USA, 2003, 100, 11594-11599; Ortmann, R. A., and Shevach, E. M. Clin. Immunol., 2001, 98, 109-118; Watford et al., Immunol. Rev., 2004, 202:139).

There remains a need for compounds and methods for the treatment of autoimmune diseases, inflammatory diseases, organ, tissue and cell transplant rejection, and hematologic disorders and malignancies.

### SUMMARY OF THE INVENTION

It has now been found that 5,7-substituted-imidazo[1,2-c]pyrimidine compounds are inhibitors of one or more JAK kinases and are useful for treating autoimmune diseases, inflammatory diseases, rejection of transplanted organs, tissues and cells, as well as hematologic disorders and malignancies and their co-morbidities.

More specifically, one aspect of the present invention provides compounds of Formula I:

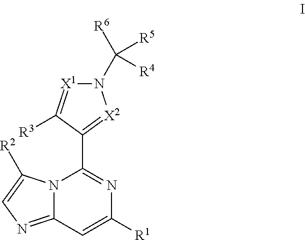

and stereoisomers and pharmaceutically acceptable salts and solvates thereof, wherein R¹, R², R³, R⁴, R⁵, R⁶, X¹ and X² are as defined herein.

Another aspect of the present invention provides compounds of Formula IA:

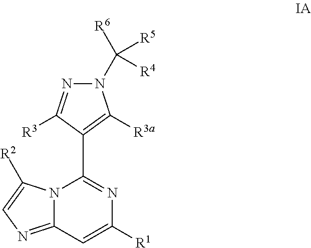

and stereoisomers and pharmaceutically acceptable salts and solvates thereof, wherein R¹, R², R³, R^{3a}, R⁴, R⁵, and R⁶ are as defined herein.

Another aspect of the present invention provides methods of treating a disease or disorder modulated by one or more JAK kinases, comprising administering to a mammal in need of such treatment an effective amount of a compound of this invention or pharmaceutically acceptable salt or solvate thereof. In one embodiment, the disease or disorder is selected from autoimmune diseases, inflammatory diseases, and organ, tissue and cell transplant rejection. In another embodiment, the disease or disorder is selected from hematological disorders and malignancies.

Another aspect of the present invention provides a pharmaceutical composition comprising a compound of the present invention or a pharmaceutically acceptable salt or solvate thereof, and a pharmaceutically acceptable carrier, diluent or excipient.

Another aspect of the present invention provides compounds of the present invention for use in therapy.

Another aspect of the present invention provides compounds of the present invention for use in the treatment of diseases or disorders selected from autoimmune diseases, inflammatory diseases, and organ, tissue and cell transplant rejection.

Another aspect of the present invention provides compounds of the present invention for use in the treatment of hematological disorders and malignancies.

Another aspect of the present invention provides the use of a compound of this invention in the manufacture of a medicament for the treatment of diseases or disorders selected from autoimmune diseases, inflammatory diseases, and organ, tissue and cell transplant rejection.

Another aspect of the present invention provides the use of a compound of this invention in the manufacture of a medicament for the treatment of hematological disorders and malignancies.

Another aspect of the present invention provides intermediates for preparing compounds of Formula I.

Another aspect of the present invention includes methods of preparing, methods of separation, and methods of purification of the compounds of this invention.

### DETAILED DESCRIPTION OF THE INVENTION

Provided herein are compounds, and pharmaceutical compositions thereof, which are useful in the treatment of diseases and disorders selected from autoimmune diseases, inflammatory diseases, organ, tissue and cell transplant rejection, and hematological disorders and malignancies.

Accordingly, one embodiment of this invention provides a compound of the general Formula I

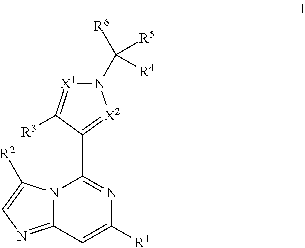

and stereoisomers and pharmaceutically acceptable salts and solvates thereof, wherein:

X¹ is N or CR^{3b};

X² is N or CR^{3a};

R¹ is hetAr¹, hetAr², hetAr³, Ar¹, Ar², (3-6C)cycloalkyl or N-(1-3C alkyl)pyridinonyl;

hetAr¹ is a 5 membered heteroaryl ring having 1-3 ring heteroatoms independently selected from N, O and S and optionally substituted with one or more substituents independently selected from halogen, (1-6C)alkyl, fluoro(1-6C)alkyl, difluoro(1-6C)alkyl, trifluoro(1-6C)alkyl, (1-4C alkoxy)(1-6C)alkyl, trimethylsilyl(1-4C alkoxy)(1-6C)alkyl, (3-6C)cycloalkyl, a 4-6 membered oxacyclic ring, hetCyc^{a}(1-2C)alkyl, hetAr^{a}(1-2C)alkyl and (1-4C alkylsulfonyl)(1-6C alkyl);

hetCyc^{a} is a 6 membered heterocycle having 1-2 ring heteroatoms independently selected from N and O and is optionally substituted with (1-6C)alkyl;

hetAr^{a} is a 6 membered heteroaryl having 1-2 ring nitrogen atoms;

hetAr² is a 9-membered bicyclic partially unsaturated or fully unsaturated heterocyclic ring having 3 ring nitrogen atoms and optionally substituted with one or more substituents independently selected from (1-6C)alkyl;

hetAr³ is a 6 membered heteroaryl having 1-2 ring nitrogen atoms and optionally substituted with one or more substituents independently selected from (1-6C)alkyl, hetCyc^{b} and (1-6C)alkoxy;

hetCyc^{b} is a 6-membered heterocycle having 1-2 ring nitrogen atoms and optionally substituted with one or more substituents independently selected from (1-6C)alkyl;

Ar¹ is phenyl substituted with a substituent selected from halogen, hetCyc^{c}, hetCyc^{d}, hetAr^{b}, trifluoro(1-6C)alkyl and (1-6C)alkoxy;

hetCyc^{c} is a 6 membered heterocycle having 1-2 ring heteroatoms independently selected from N and O and optionally substituted with one or more substituents independently selected from (1-6C)alkyl;

hetCyc^{d} is an 8-membered bridged heterocyclic ring having 1-2 ring heteroatoms independently selected from N and O;

hetAr^{b} is a 5-membered heteroaryl ring having 1-2 ring nitrogen atoms and optionally substituted with one or more substituents independently selected from (1-6C)alkyl;

Ar² is a benzo ring fused to a 5-6 membered azacyclic ring and is optionally substituted with one or more substituents independently selected from (1-6C)alkyl;

R² is hydrogen, halogen, (1-4C)alkyl, CF₃, CN, or (3-4C)cycloalkyl;

R³, R^{3a} and R^{3b} are independently hydrogen, (1-6C)alkyl, CF₃, F, Cl, CN or (3-6C)cycloalkyl;

R⁴ is hydrogen, and

R⁵ is hydrogen, (3-6C)cycloalkyl (optionally substituted by one or more halogens), (3-6C)cycloalkylCH₂— (optionally substituted by one or more halogens), (1-6C)alkyl, a 4-6 membered heterocycle having 1-2 ring heteroatoms independently selected from N, O and S, or phenyl optionally substituted with one or more halogens,

or R⁴ and R⁵ together with the carbon atom to which they are attached form a 4- or 5-membered azacyclic ring substituted with a substituent selected from fluoro(1-6C)alkyl, difluoro (1-6C)alkyl, trifluoro (1-6C)alkyl, (1-6Calkyl)C(═O)O—, —SO₂R^{c}, (1-6C)alkyl, (1-6Calkyl)C(═O)—, phenylC(═O)—, cyclopropyl-C(═O)—, (1-6C alkyl)NHC(═O)—, di(1-6C alkyl)NC(═O)—, or cyano(1-6Calkyl),

or R⁴ and R⁵ together with the carbon atom to which they are attached form a 3-6-membered carbocyclic ring optionally substituted with one or more substitutents independently selected from methyl and halogen;

R^{c} is H, fluoro (1-3C)alkyl, difluoro (1-3C)alkyl trifluoro (1-3C)alkyl, (3-6C)cycloalkyl, cyclopropylamino, cyclopropylmethyl, (1-6C)alkyl, or a 5-membered heteroaryl having 1-2 ring heteroatoms independently selected from N, O and S, wherein said 5-membered heteroaryl is optionally substituted with one or more substituents independently selected from (1-6C)alkyl; and

R⁶ is H, (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, hydroxy(1-6C)alkyl, (1-3C alkoxy)(1-6C)alkyl, (1-3C alkylsufanyl)(1-3C)alkyl, (1-3C alkyl)OC(═O)(1-3C)alkyl, carboxy(1-6C)alkyl, fluoro(2-6C)alkenyl, difluoro(2-6C)alkenyl or (1-6C)alkylC(═O)CH₂—.

In one embodiment, compounds of Formula B include compounds of the general Formula IA

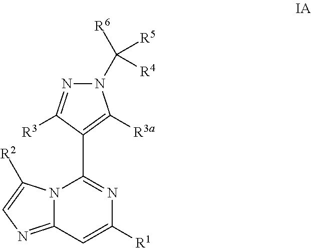

and stereoisomers and pharmaceutically acceptable salts and solvates thereof, wherein:

R¹ is hetAr¹, hetAr², hetAr³, Ar¹, Ar², (3-6C)cycloalkyl or N-(1-3C alkyl)pyridinonyl;

hetAr¹ is a 5 membered heteroaryl ring having 1-3 ring heteroatoms independently selected from N, O and S and optionally substituted with one or more substituents independently selected from halogen, (1-6C)alkyl, fluoro(1-6C)alkyl, difluoro(1-6C)alkyl, trifluoro(1-6C)alkyl, (1-4C alkoxy)(1-6C)alkyl, trimethylsilyl(1-4C alkoxy)(1-6C)alkyl, (3-6C)cycloalkyl, a 4-6 membered oxacyclic ring, hetCyc^{a}(1-2C)alkyl, hetAr^{a}(1-2C)alkyl and (1-4C alkylsulfonyl)(1-6C alkyl);

hetCyc^{a} is a 6 membered heterocycle having 1-2 ring heteroatoms independently selected from N and O and is optionally substituted with (1-6C)alkyl;

hetAr^{a} is a 6 membered heteroaryl having 1-2 ring nitrogen atoms;

hetAr² is a 9-membered bicyclic partially unsaturated or fully unsaturated heterocyclic ring having 3 ring nitrogen atoms and optionally substituted with one or more substituents independently selected from (1-6C)alkyl;

hetAr³ is a 6 membered heteroaryl having 1-2 ring nitrogen atoms and optionally substituted with one or more substituents independently selected from (1-6C)alkyl, hetCyc^{b} and (1-6C)alkoxy;

hetCyc^{b} is a 6-membered heterocycle having 1-2 ring nitrogen atoms and optionally substituted with one or more substituents independently selected from (1-6C)alkyl;

Ar¹ is phenyl substituted with a substituent selected from halogen, hetCyc^{c}, hetCyc^{d}, hetAr^{b}, trifluoro(1-6C)alkyl and (1-6C)alkoxy;

hetCyc^{c} is a 6 membered heterocycle having 1-2 ring heteroatoms independently selected from N and O and optionally substituted with one or more substituents independently selected from (1-6C)alkyl;

hetCyc^{d} is an 8-membered bridged heterocyclic ring having 1-2 ring heteroatoms independently selected from N and O;

hetAr^{b} is a 5-membered heteroaryl ring having 1-2 ring nitrogen atoms and optionally substituted with one or more substituents independently selected from (1-6C)alkyl;

Ar² is a benzo ring fused to a 5-6 membered azacyclic ring and is optionally substituted with one or more substituents independently selected from (1-6C)alkyl;

R² is hydrogen, halogen, (1-4C)alkyl, CF₃, CN, or (3-4C)cycloalkyl;

R³ and R^{3a} are independently hydrogen, (1-6C)alkyl, CF₃, F, Cl, CN or (3-6C)cycloalkyl;

R⁴ is hydrogen, and

R⁵ is hydrogen, (3-6C)cycloalkyl (optionally substituted by one or more halogens), or (3-6C)cycloalkylCH₂— (optionally substituted by one or more halogens),

or R⁴ and R⁵ together with the carbon atom to which they are attached form a 4- or 5-membered azacyclic ring substituted with a substituent selected from fluoro(1-6C)alkyl, difluoro(1-6C)alkyl, trifluoro(1-6C)alkyl, (1-6Calkyl)C(═O)O— and —SO₂R^{c};

R^{c} is fluoro(1-3C)alkyl, difluoro(1-3C)alkyl trifluoro(1-3C)alkyl, or (3-6C)cycloalkyl; and

R⁶ is (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, hydroxy(1-6C)alkyl, (1-3C alkoxy)(1-6C)alkyl, (1-3C alkylsufanyl)(1-3C)alkyl, (1-3C alkyl)OC(═O)(1-3C)alkyl, or carboxy(1-6C)alkyl.

In one embodiment of Formula I, R¹ is hetAr¹, wherein hetAr¹ is a 5 membered heteroaryl ring having 1-3 ring heteroatoms independently selected from N, O and S and optionally substituted with one or more substituents independently selected from halogen, (1-6C)alkyl, fluoro(1-6C)alkyl, difluoro(1-6C)alkyl, trifluoro(1-6C)alkyl, (1-4C alkoxy)(1-6C)alkyl, trimethylsilyl(1-4C alkoxy)(1-6C)alkyl, (3-6C)cycloalkyl, a 4-6 membered oxacyclic ring, hetCyc^{a}(1-2C)alkyl, hetAr^{a}(1-2C)alkyl and (1-4C alkylsulfonyl)(1-6C alkyl). In one embodiment, hetAr¹ is substituted with one or two of said substituents. In one embodiment, hetAr¹ is substituted with one of said substituents.

Particular examples of halogen substituents for hetAr¹ include F, Cl, and Br.

Particular examples of (1-6C)alkyl substituents for hetAr¹ include methyl, ethyl, propyl, isopropyl, butyl, isobutyl, and tert-butyl.

Particular examples of fluoro(1-6C)alkyl substituents for hetAr¹ include fluoromethyl and fluoroethyl.

Particular examples of difluoro(1-6C)alkyl substituents for hetAr¹ include difluoromethyl and difluoroethyl.

Particular examples of trifluoro(1-6C)alkyl substituents for hetAr¹ include trifluoromethyl and 2,2,2-trifluoroethyl.

Particular examples of (1-4C alkoxy)(1-6C)alkyl substituents for hetAr¹ include methoxymethyl, ethoxyethyl, ethoxyethyl, (2-isopropoxy)ethyl, methoxymethyl, and 2-methoxyprop-2-yl. In one embodiment, the (1-4C alkoxy)(1-6C)alkyl substituents are selected from methoxymethyl, ethoxyethyl, ethoxyethyl and (2-isopropoxy)ethyl.

A particular example of a trimethylsilyl(1-4C alkoxy)(1-6C)alkyl substituent for hetAr¹ is trimethylsilylethoxymethyl.

Particular examples of (3-6C)cycloalkyl substituents for hetAr¹ include cyclopropyl, cyclobutyl, cyclopentyl and cyclohexyl.

Particular examples of 4-6 membered oxacyclic ring substituents for hetAr¹ include oxetanyl, tetrahydrofuranyl and tetrahydropyranyl groups.

Particular examples of hetCyc^{a}(1-2C)alkyl substituents for hetAr¹ include piperidinylmethyl, piperidinylethyl, piperazinylmethyl, piperazinylmethyl and morpholinylmethyl. A particular example is (4-methylpiperazinyl)ethyl.

Particular examples of hetAr^{a}(1-2C)alkyl substituents for hetAr¹ include pyridinylmethyl, pyridinylethyl, pyrimidinylmethyl and pyrimidinylethyl. A particular example is pyrid-3-ylmethyl.

Particular examples of (1-4C alkylsulfonyl)(1-6C alkyl) substituents for hetAr¹ include CH₃SO₂(1-6C alkyl), for example CH₃SO₂CH₂CH₂—.

In one embodiment, hetAr¹ is pyrazolyl, thiazolyl, oxazolyl, thiadiazolyl, imidazolyl, pyrrolyl or thiophenyl optionally substituted with one or more substituents independently selected from halogen, (1-6C)alkyl, fluoro(1-6C)alkyl, difluoro(1-6C)alkyl, trifluoro(1-6C)alkyl, (1-4C alkoxy)(1-6C)alkyl, trimethylsilyl(1-4C alkoxy)(1-6C)alkyl, (3-6C)cycloalkyl, a 4-6 membered oxacyclic ring, hetCyc^{a}(1-2C)alkyl, hetAr^{a}(1-2C)alkyl and (1-4C alkylsulfonyl)(1-6C alkyl).

In one embodiment, hetAr¹ is pyrazolyl, thiazolyl, oxazolyl, thiadiazolyl or imidazolyl optionally substituted with one or more substituents independently selected from halogen, (1-6C)alkyl, fluoro(1-6C)alkyl, difluoro(1-6C)alkyl, trifluoro(1-6C)alkyl, (1-4C alkoxy)(1-6C)alkyl, trimethylsilyl(1-4C alkoxy)(1-6C)alkyl, (3-6C)cycloalkyl, a 4-6 membered oxacyclic ring, hetCyc^{a}(1-2C)alkyl, hetAr^{a}(1-2C)alkyl and (1-4C alkylsulfonyl)(1-6C alkyl).

In one embodiment, hetAr¹ is pyrazol-4-yl, thiazol-5-yl, imidazol-1-yl or 1,3,4-thiadiazol-2-yl optionally substituted with one or more substituents independently selected from halogen, (1-6C)alkyl, fluoro(1-6C)alkyl, difluoro(1-6C)alkyl, trifluoro(1-6C)alkyl, (1-4C alkoxy)(1-6C)alkyl, trimethylsilyl(1-4C alkoxy)(1-6C)alkyl, (3-6C)cycloalkyl, a 4-6 membered oxacyclic ring, hetCyc^{a}(1-2C)alkyl, hetAr^{a}(1-2C)alkyl and (1-4C alkylsulfonyl)(1-6C alkyl).

In one embodiment, hetAr¹ is pyrazol-4-yl, thiazol-5-yl, or imidazol-1-yl optionally substituted with one or more substituents independently selected from halogen, (1-6C)alkyl, fluoro(1-6C)alkyl, difluoro(1-6C)alkyl, trifluoro(1-6C)alkyl, (1-4C alkoxy)(1-6C)alkyl, trimethylsilyl(1-4C alkoxy)(1-6C)alkyl, (3-6C)cycloalkyl, a 4-6 membered oxacyclic ring, hetCyc^{a}(1-2C)alkyl, hetAr^{a}(1-2C)alkyl and (1-4C alkylsulfonyl)(1-6C alkyl).

In one embodiment, hetAr¹ is pyrazol-4-yl, thiazol-5-yl, imidazol-1-yl or 1,3,4-thiadiazol-2-yl optionally substituted with one or more substituents independently selected from F, Cl, Br, methyl, ethyl, isopropyl, isobutyl, 2,2,2-trifluoroethyl, (2-isopropoxy)ethyl, trimethylsilylethoxymethyl, cyclobutyl, 4-tetrahydro-2H-pyranyl, (4-methylpiperazinyl)ethyl, pyrid-3-ylmethyl and CH₃SO₂CH₂CH₂—.

In one embodiment, hetAr¹ is pyrazol-4-yl, thiazol-5-yl or imidazol-1-yl optionally substituted with one or more substituents independently selected from F, Cl, Br, methyl, ethyl, isopropyl, isobutyl, 2,2,2-trifluoroethyl, (2-isopropoxy)ethyl, trimethylsilylethoxymethyl, cyclobutyl, 4-tetrahydro-2H-pyranyl, (4-methylpiperazinyl)ethyl, pyrid-3-ylmethyl and CH₃SO₂CH₂CH₂—.

In one embodiment, hetAr¹ is pyrazol-4-yl optionally substituted a substituent selected from halogen, (1-6C)alkyl, fluoro(1-6C)alkyl, difluoro(1-6C)alkyl, trifluoro(1-6C)alkyl, (1-4C alkoxy)(1-6C)alkyl, trimethylsilyl(1-4C alkoxy)(1-6C)alkyl, (3-6C)cycloalkyl, a 4-6 membered oxacyclic ring, hetCyc^{a}(1-2C)alkyl, hetAr^{a}(1-2C)alkyl and (1-4C alkylsulfonyl)(1-6C alkyl).

In one embodiment, hetAr¹ is pyrazol-4-yl optionally substituted a substituent selected from F, Cl, Br, methyl, ethyl, isopropyl, isobutyl, 2,2,2-trifluoroethyl, (2-isopropoxy)ethyl, trimethylsilylethoxymethyl, cyclobutyl, oxetanyl, 4-tetrahydro-2H-pyranyl, (4-methylpiperazinyl)ethyl, pyrid-3-ylmethyl and CH₃SO₂CH₂CH₂—.

In one embodiment, hetAr¹ is pyrazol-4-yl optionally substituted a substituent selected from methyl, ethyl, isopropyl, isobutyl, 2,2,2-trifluoroethyl, (2-isopropoxy)ethyl, trimethylsilylethoxymethyl, and cyclobutyl.

In one embodiment, hetAr¹ is pyrazol-4-yl optionally substituted a substituent selected from methyl, ethyl, isopropyl, isobutyl and 2,2,2-trifluoroethyl.

In one embodiment, hetAr¹ is pyrazol-4-yl optionally substituted with a substituent selected from (1-6C)alkyl. In one embodiment, hetAr¹ is pyrazol-4-yl optionally substituted with methyl.

Particular examples of R¹ when represented by hetAr¹ include the structures:

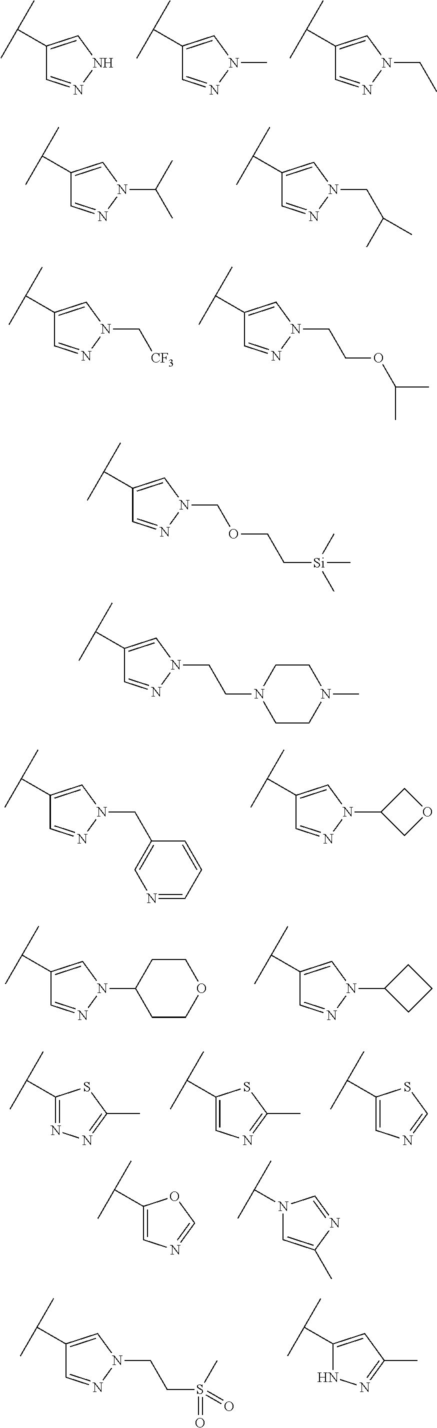

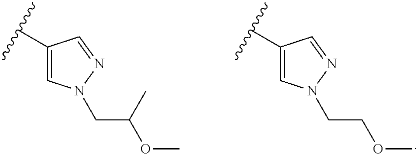

In one embodiment, R¹ is hetAr², wherein hetAr² is a 9-membered bicyclic partially unsaturated or fully unsaturated heterocyclic ring having 3 ring nitrogen atoms and optionally substituted with one or more substituents independently selected from (1-6C)alkyl.

In one embodiment, hetAr² is 5,6,7,8-tetrahydroimidazo[1,2-a]pyrazinyl optionally substituted with one or more substituents independently selected from (1-6C)alkyl, such as methyl or ethyl.

Particular examples of R¹ when represented by hetAr² include the structures:

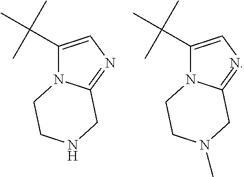

In one embodiment, R¹ is hetAr³, wherein hetAr³ is a 6 membered heteroaryl having 1-2 ring nitrogen atoms and optionally substituted with one or more substituents independently selected from (1-6C)alkyl, hetCyc^{b} and (1-6C)alkoxy.

In one embodiment, hetAr³ is pyridyl or pyrimidyl optionally substituted with one or more substituents independently selected from (1-6C)alkyl, hetCyc^{b} and (1-6C)alkoxy.

Examples of (1-6C)alkyl substituents for hetAr³ include methyl and ethyl.

Examples of hetCyc^{b} substituents for hetAr³ include piperidinyl and piperazinyl rings optionally substituted with one or more substituents independently selected from (1-6C)alkyl, such as methyl or ethyl. A particular example of hetCyc^{b} includes 4-methylpiperazinyl.

Examples of (1-6C)alkoxy substituents for hetAr³ include methoxy and ethoxy.

In one embodiment, hetAr³ is pyridyl optionally substituted with methyl, 4-methylpiperazinyl or methoxy.

Particular examples of R¹ when represented by hetAr³ include the structures:

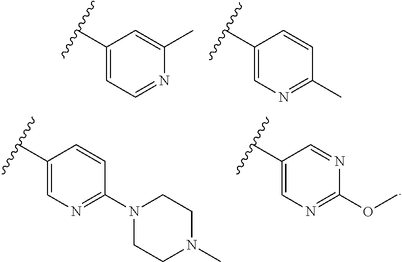

In one embodiment, R¹ is Ar¹, wherein Ar¹ is phenyl substituted with a substituent selected from halogen, hetCyc^{c}, hetCyc^{d}, hetAr^{b}, trifluoro(1-6C)alkyl and (1-6C)alkoxy.

Particular examples of halogen substituents for Ar¹ include F, Cl and Br.

In one embodiment, Ar¹ is phenyl substituted with hetCyc^{c}, wherein hetCyc^{c} is a 6 membered heterocycle having 1-2 ring heteroatoms independently selected from N and O and optionally substituted with one or more substituents independently selected from (1-6C)alkyl. Examples of hetCyc^{c} include piperidinyl, piperazinyl and morpholinyl rings optionally substituted with one or more substituents independently selected from (1-6C)alkyl, for example methyl and ethyl. Particular examples of hetCyc^{c} include 1-methylpiperidin-4-yl, 1-methylpiperazin-4-yl and morpholinyl.

In one embodiment, Ar¹ is phenyl substituted with hetCyc^{d}, where hetCyc^{d} is an 8-membered bridged heterocyclic ring having 1-2 ring heteroatoms independently selected from N and O. An example of hetCyc^{d} is 8-oxa-3-azabicyclo[3.2.1]octanyl.

In one embodiment, Ar¹ is phenyl substituted with hetAr^{b}, wherein hetAr^{b} is a 5-membered heteroaryl ring having 1-2 ring nitrogen atoms and optionally substituted with one or more substituents independently selected from (1-6C)alkyl. Examples of hetAr^{b} include pyrrolyl and pyrazolyl rings optionally substituted with one or more substituents independently selected from (1-6C)alkyl, for example methyl and ethyl. A particular example of hetAr^{b} is 1-methylpyrazol-3-yl.

In one embodiment, Ar¹ is phenyl optionally substituted with a substituent selected from (i) morpholinyl, (ii) piperidinyl optionally substituted with (1-6C)alkyl, (iii) piperazinyl optionally substituted with (1-6C)alkyl, (iv) oxa-3-azabicyclo[3.2.1]octane, (v) pyrazolyl optionally substituted with (1-6C)alkyl, (vi) trifluoro(1-6C)alkyl, and (vi) (1-6C)alkoxy.

In one embodiment Ar¹ is phenyl substituted with a substituent selected from morpholin-4-yl, 1-methylpiperidin-4-yl, 1-methylpiperazin-4-yl, 8-oxa-3-azabicyclo[3.2.1 ]octanyl, 1-methyl-1H-pyrazolyl, methoxy or trifluoromethyl.

In one embodiment, Ar¹ is phenyl substituted with trifluoro(1-6C)alkyl or (1-6C)alkoxy. In one embodiment, Ar¹ is phenyl substituted with methoxy or trifluoromethyl.

Particular examples of R¹ when represented by Ar¹ include the structures:

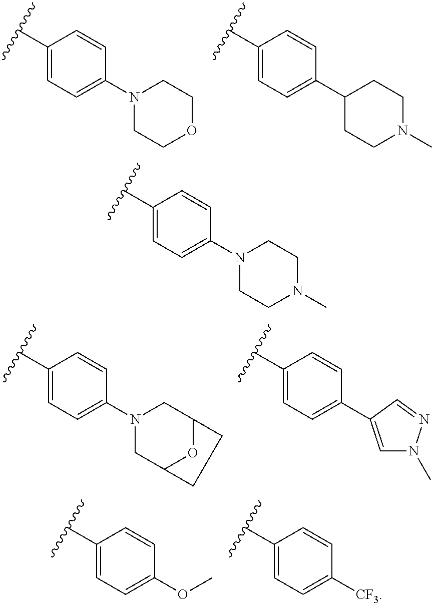

In one embodiment, R¹ is Ar², wherein Ar² is a benzo ring fused to a 5-6 membered azacyclic ring and is optionally substituted with one or more substituents independently selected from (1-6C)alkyl, such as methyl or ethyl. In one embodiment, Ar² is 1,2,3,4-tetrahydroisoquinolin-6-yl or 1,2,3,4-tetrahydroisoquinolin-7-yl optionally substituted with one or more substituents independently selected from (1-6C)alkyl. Particular examples of R¹ when represented by Ar² include the structures:

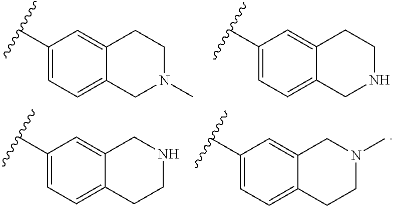

In one embodiment, R¹ is selected from hetAr¹, hetAr², hetAr³, Ar¹ and Ar².

In one embodiment, R¹ is selected from hetAr¹ and hetAr².

In one embodiment, R¹ is selected from Ar¹ and Ar².

In one embodiment, R¹ is N-(1-3C alkyl)pyridinonyl. In one embodiment, R¹ is N-methylpyridonyl. In one embodiment, R¹ is 1-methylpyridin-2(1H)-on-5-ly or 1-dimethylpyridin-2(1H)-one-4-yl, which can be represented by the structures:

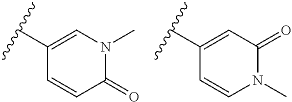

respectively.

In one embodiment, R¹ is (3-6C) cycloalkyl. In one embodiment, R¹ is cyclopropyl.

In one embodiment, R² is hydrogen.

In one embodiment, R² is halogen. In one embodiment, R² is F, Cl or Br. In one embodiment, R² is F or Cl. In one embodiment, R² is F. In one embodiment, R² is Cl.

In one embodiment of Formula I, R² is (1-4C)alkyl. In one embodiment, R² is methyl, ethyl, propyl, isopropyl, butyl, isobutyl or tert-butyl. In one embodiment of Formula I, R² is (1-3C)alkyl. In one embodiment, R² is methyl.

In one embodiment of Formula I, R² is CF₃.

In one embodiment of Formula I, R² is CN.

In one embodiment of Formula I, R² is (3-4C)cycloalkyl. In one embodiment of Formula I, R² is cyclopropyl.

In one embodiment of Formula I, R² is selected from hydrogen, halogen, (1-4C)alkyl, CF₃ and CN.

In one embodiment of Formula I, R² is selected from hydrogen, F, Cl, methyl, CF₃ and CN.

In one embodiment of Formula I, R² is hydrogen, F, Cl, Br, methyl or CN.

In one embodiment of Formula I, R² is hydrogen, F, Cl or CN.

In one embodiment of Formula I, R² is hydrogen, Cl or CN.

In one embodiment, R³ is hydrogen.

In one embodiment, R³ is (1-6C)alkyl. A particular example is methyl.

In one embodiment, R³ is CF₃.

In one embodiment, R³ is F.

In one embodiment, R³ is Cl.

In one embodiment, R³ is CN.

In one embodiment, R³ is (3-6C)cycloalkyl. In one embodiment, R³ is cyclopropyl.

In one embodiment, R³ is hydrogen or methyl.

In one embodiment, R³ is selected from hydrogen, (1-6C)alkyl, CF₃, F and Cl.

In one embodiment, R³ is selected from hydrogen, methyl, F and Cl.

In one embodiment, X¹ is N and X² is CR^{3a}, such that the residue at the 5-position of the imidazo[1,2-c]pyrimidine ring has the structure shown as structure A:

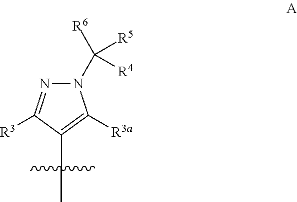

wherein the wavy line indicates the point of attachment to the 5-position of the imidazo[1,2-c]pyrimidine ring and R³, R^{3a}, R⁴, R⁵ and R⁶ are as defined for Formula I.

In one embodiment of structure A, R^{3a} is hydrogen.

In one embodiment of structure A, R^{3a} is (1-6C)alkyl. A particular example is methyl.

In one embodiment of structure A, R^{3a} is CF₃.

In one embodiment of structure A, R^{3a} is F.

In one embodiment of structure A, R^{3a} is Cl.

In one embodiment of structure A, R^{3a} is CN.

In one embodiment of structure A, R^{3a} is (3-6C)cycloalkyl. In one embodiment, R^{3a} is cyclopropyl.

In one embodiment of structure A, R³ and R^{3a} are independently selected from hydrogen, (1-6C alkyl), CF₃, F, and Cl. In one embodiment, R³ and R^{3a} are independently selected from hydrogen, F, Cl, CF₃ and methyl. In one embodiment, R³ and R^{3a} are independently selected from hydrogen and (1-6C alkyl). In one embodiment, R³ and R^{3a} are independently selected from hydrogen and methyl.

In one embodiment of structure A, R³ and R^{3a} are both hydrogen.

In one embodiment, X¹ is CR^{3b} and X² is CR^{3a}, such that the group at the 5-position of the imidazo[1,2-c]pyrimidine ring has the structure shown as structure B:

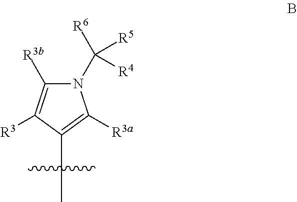

wherein the wavy line indicates the point of attachment to the 5-position of the imidazo[1,2-c]pyrimidine ring and R³, R^{3a}, R^{3b}, R⁴, R⁵ and R⁶ are as defined for Formula I.

In one embodiment of structure B, R³, R^{3a} and R^{3b} are hydrogen.

In one embodiment, X¹ is CR^{3b} and X² is N, such that the residue at the 5-position of the imidazo[1,2-c]pyrimidine ring has the structure shown as structure C:

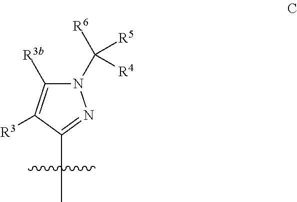

wherein the wavy line indicates the point of attachment to the 5-position of the imidazo[1,2-c]pyrimidine ring and R³, R^{3b}, R⁴, R⁵ and R⁶ are as defined for Formula I.

In one embodiment of structure C, R³ and R^{3b} are hydrogen.

In one embodiment, X¹ is N and X² is N, such that the residue at the 5-position of the imidazo[1,2-c]pyrimidine ring has the structure shown as structure D:

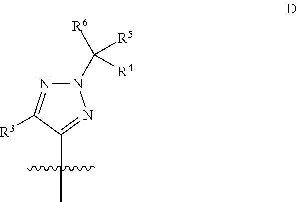

wherein the wavy line indicates the point of attachment to the 5-position of the imidazo[1,2-c]pyrimidine ring and R³, R⁴, R⁵ and R⁶ are as defined for Formula I.

In one embodiment of structure D, R³ is hydrogen.

In one embodiment of Formula I, R⁴ is hydrogen and R⁵ is hydrogen, (3-6C)cycloalkyl (optionally substituted by one or more halogens), (3-6C)cycloalkylCH₂-(optionally substituted by one or more halogens), (1-6C)alkyl, a 5-6 membered heterocycle having 1-2 ring heteroatoms independently selected from N, O and S, or phenyl optionally substituted with one or more halogens.

In one embodiment, R⁴ is hydrogen and R⁵ is hydrogen, (3-6C)cycloalkyl (optionally substituted by one or more halogens), or (3-6C)cycloalkylCH₂— (optionally substituted by one or more halogens).

In one embodiment, R⁴ is hydrogen and R⁵ is hydrogen.

In one embodiment, R⁴ is hydrogen and R⁵ is (3-6C)cycloalkyl optionally substituted with one or more halogens. In one embodiment, R⁴ is hydrogen and R⁵ is (3-6C)cycloalkyl optionally substituted with one or more fluorines. In one embodiment, R⁴ is hydrogen and R⁵ is cyclopropyl, 2,2-difluorocyclopropyl, cyclobutyl, cyclopentyl or cyclohexyl. In one embodiment, R⁴ is hydrogen and R⁵ is cyclopropyl.

In one embodiment, R⁴ is hydrogen and R⁵ is (3-6C)cycloalkylCH₂— which is optionally substituted with one or more halogens. In one embodiment, R⁴ is hydrogen and R⁵ is (3-6C)cycloalkylCH₂— which is optionally substituted with one or more fluorines. In one embodiment, R⁴ is hydrogen and R⁵ is cyclopropylmethyl.

In one embodiment, R⁴ is hydrogen and R⁵ is (1-6C)alkyl. In one embodiment, R⁴ is hydrogen and R⁵ is ethyl.

In one embodiment, R⁴ is hydrogen and R⁵ is a 4-6 membered heterocycle having 1-2 ring heteroatoms independently selected from N, O and S. In one embodiment, R⁴ is hydrogen and R⁵ is a 5-membered heterocycle having 1-2 ring heteroatoms independently selected from N, O and S. In one embodiment, R⁴ is hydrogen and R⁵ is tetrahydropyranyl.

In one embodiment, R⁴ is hydrogen and R⁵ is phenyl optionally substituted with one or more halogens. In one embodiment, R⁴ is hydrogen and R⁵ is phenyl optionally substituted with one or more fluorines.

In one embodiment, R⁴ is hydrogen and R⁵ is hydrogen, cyclopropyl or cyclopropylmethyl.

In one embodiment, R⁴ and R⁵ together with the carbon atom to which they are attached form a 4- or 5-membered azacyclic ring substituted with a substituent selected from fluoro(1-6C)alkyl, difluoro(1-6C)alkyl, trifluoro(1-6C)alkyl, (1-6Calkyl)C(═O)O—, —SO₂R^{c}, (1-6C)alkyl, (1-6Calkyl)C(═O)—, phenylC(═O)—, cyclopropyl-C(═O)—, (1-6C alkyl)NHC(═O)—, di(1-6C alkyl)NC(═O)—, or cyano(1-6Calkyl).

In one embodiment, R⁴ and R⁵ together with the carbon atom to which they are attached form a 4-membered azacyclic ring substituted with a substituent selected from fluoro(1-6C)alkyl, difluoro(1-6C)alkyl, trifluoro(1-6C)alkyl, (1-6C alkyl)C(═O)O— and —SO₂R^{c}. In one embodiment, the substituent is coupled to the nitrogen atom of the 4-membered azacyclic ring.

In one embodiment, R⁴ and R⁵ together with the carbon atom to which they are attached form a 4-membered azacyclic ring substituted with a substituent selected from fluoro(1-6C)alkyl, difluoro(1-6C)alkyl and trifluoro(1-6C)alkyl. In one embodiment, R⁴ and R⁵ together with the carbon atom to which they are attached form a 4-membered azacyclic ring substituted with a substituent selected from fluoromethyl, 3-fluoropropyl, 2-fluoroethyl, 2,2-difluoroethyl, 1,3-difluoroprop-2-yl, 2,2,2-trifluoroethyl, and 3,3,3-trifluoropropyl. In one embodiment, the substituent is coupled to the nitrogen atom of the 4-membered azacyclic ring.

In one embodiment, R⁴ and R⁵ together with the carbon atom to which they are attached form a 4-membered azacyclic ring substituted with a substituent selected from (1-6Calkyl)C(═O)O—. In one embodiment, R⁴ and R⁵ together with the carbon atom to which they are attached form a 4-membered azacyclic ring substituted with (CH₃)₃CC(═O)O—. In one embodiment, the substituent is coupled to the nitrogen atom of the 4-membered azacyclic ring.

In one embodiment, R⁴ and R⁵ together with the carbon atom to which they are attached form a 4-membered azacyclic ring substituted with —SO₂R^{c}, where R^{c} is H, fluoro(1-3C)alkyl, difluoro(1-3C)alkyl trifluoro(1-3C)alkyl, (3-6C)cycloalkyl, cyclopropylamino, cyclopropylmethyl, (1-6C)alkyl, or a 5-membered heteroaryl having 1-2 ring heteroatoms independently selected from N, O and S. In one embodiment, the substituent is coupled to the nitrogen atom of the 4-membered azacyclic ring. In one embodiment, R⁴ and R⁵ together with the carbon atom to which they are attached form a 4-membered azacyclic ring substituted with —SO₂CH₃, —SO₂CH₂CH₃, —SO₂CH₂CH₂CH₃, —SO₂CH(CH₃)₂, SO₂CHH₂CF₃, —SO₂CF₃, —SO₂CF₂CF₃, SO₂CF₂H, SO₂CH₂CF₃, —SO₂-cyclopropyl, cyclpropylamino, cyclopropylmethyl, methyl, isopropyl, or a pyrazolyl group optionally substituted with one or more methyls.

In one embodiment, R⁴ and R⁵ together with the carbon atom to which they are attached form a 4-membered azacyclic ring substituted with —SO₂R^{c}, where R^{c} is fluoro(1-3C)alkyl, difluoro(1-3C)alkyl trifluoro(1-3C)alkyl, or (3-6C)cycloalkyl. In one embodiment, the substituent is coupled to the nitrogen atom of the 4-membered azacyclic ring. In one embodiment, R⁴ and R⁵ together with the carbon atom to which they are attached form a 4-membered azacyclic ring substituted with —SO₂CH₃, —SO₂CH₂CH₃, —SO₂CH₂CH₂CH₃, —SO₂CH(CH₃)₂, —SO₂CHH₂CF₃, —SO₂CF₃, —SO₂CF₂CF₃, SO₂CF₂H, SO₂CH₂CF₃ or —SO₂-cyclopropyl. In one embodiment, R⁴ and R⁵ together with the carbon atom to which they are attached form a 4-membered azacyclic ring substituted with —SO₂CF₃, SO₂CF₂H or —SO₂-cyclopropyl. In one embodiment, the substituent is coupled to the nitrogen atom of the 4-membered azacyclic ring.

In one embodiment, R⁴ and R⁵ together with the carbon atom to which they are attached form a 4-membered azacyclic ring substituted with a substituent selected from fluoromethyl, 3-fluoropropyl, 2-fluoroethyl, 2,2-difluoroethyl, 1,3-difluoroprop-2-yl, 2,2,2-trifluoroethyl, 3,3,3-trifluoropropyl, SO₂CH₃, SO₂CH₂CH₃, SO₂CH₂CH₂CH₃, SO₂CH(CH₃)₂, SO₂CH₂CH₂CF₃, SO₂CF₃, SO₂CF₂CF₃, SO₂CF₂H and —SO₂cyclopropyl. In one embodiment, the substituent is coupled to the nitrogen atom of the 4-membered azacyclic ring.

In one embodiment, R⁴ and R⁵ together with the carbon atom to which they are attached form a 4-membered azacyclic ring substituted with (1-6C)alkyl. In one embodiment, R⁴ and R⁵ together with the carbon atom to which they are attached form a 4-membered azacyclic ring substituted with ethyl. In one embodiment, the substituent is coupled to the nitrogen atom of the 4-membered azacyclic ring.

In one embodiment, R⁴ and R⁵ together with the carbon atom to which they are attached form a 4-membered azacyclic ring substituted with (1-6Calkyl)C(═O)—. In one embodiment, R⁴ and R⁵ together with the carbon atom to which they are attached form a 4-membered azacyclic ring substituted with CH₃C(═O)—. In one embodiment, the substituent is coupled to the nitrogen atom of the 4-membered azacyclic ring.

In one embodiment, R⁴ and R⁵ together with the carbon atom to which they are attached form a 4-membered azacyclic ring substituted with phenylC(═O)—. In one embodiment, the substituent is coupled to the nitrogen atom of the 4-membered azacyclic ring.

In one embodiment, R⁴ and R⁵ together with the carbon atom to which they are attached form a 4-membered azacyclic ring substituted with cyclopropyl-C(═O)—. In one embodiment, the substituent is coupled to the nitrogen atom of the 4-membered azacyclic ring.

In one embodiment, R⁴ and R⁵ together with the carbon atom to which they are attached form a 4-membered azacyclic ring substituted with (1-6C alkyl)NHC(═O)—. In one embodiment, R⁴ and R⁵ together with the carbon atom to which they are attached form a 4-membered azacyclic ring substituted with CH₃CH₂NHC(═O)—. In one embodiment, the substituent is coupled to the nitrogen atom of the 4-membered azacyclic ring.

In one embodiment, R⁴ and R⁵ together with the carbon atom to which they are attached form a 4-membered azacyclic ring substituted with di(1-6C alkyl)NC(═O)—. In one embodiment, R⁴ and R⁵ together with the carbon atom to which they are attached form a 4-membered azacyclic ring substituted with Me₂NC(═O). In one embodiment, the substituent is coupled to the nitrogen atom of the 4-membered azacyclic ring.

In one embodiment, R⁴ and R⁵ together with the carbon atom to which they are attached form a 4-membered azacyclic ring substituted with cyano(1-6Calkyl). In one embodiment, R⁴ and R⁵ together with the carbon atom to which they are attached form a 4-membered azacyclic ring substituted with CNCH₂—. In one embodiment, the substituent is coupled to the nitrogen atom of the 4-membered azacyclic ring.

In one embodiment, R⁴ and R⁵ together with the carbon atom to which they are attached form a 5-membered azacyclic ring substituted with a substituent selected from fluoro(1-6C)alkyl, difluoro(1-6C)alkyl, trifluoro(1-6C)alkyl, (1-6Calkyl)C(═O)O— and —SO₂R^{c}. In one embodiment, the substituent is coupled to the nitrogen atom of the 5-membered azacyclic ring.

In one embodiment, R⁴ and R⁵ together with the carbon atom to which they are attached form a 5-membered azacyclic ring substituted with a substituent selected from fluoro(1-6C)alkyl, difluoro(1-6C)alkyl and trifluoro(1-6C)alkyl. In one embodiment, R⁴ and R⁵ together with the carbon atom to which they are attached form a 5-membered azacyclic ring substituted with a substituent selected from fluoromethyl, 3-fluoropropyl, 2-fluoroethyl, 2,2-difluoroethyl, 1,3-difluoroprop-2-yl, 2,2,2-trifluoroethyl, and 3,3,3-trifluoropropyl. In one embodiment, the substituent is coupled to the nitrogen atom of the 5-membered azacyclic ring.

In one embodiment, R⁴ and R⁵ together with the carbon atom to which they are attached form a 5-membered azacyclic ring substituted with a substituent selected from (1-6Calkyl)C(═O)O—. In one embodiment, R⁴ and R⁵ together with the carbon atom to which they are attached form a 5-membered azacyclic ring substituted with (CH₃)₃CC(═O)O—. In one embodiment, the substituent is coupled to the nitrogen atom of the 5-membered azacyclic ring.

In one embodiment, R⁴ and R⁵ together with the carbon atom to which they are attached form a 5-membered azacyclic ring substituted with —SO₂R^{c}, where R^{c} is H, fluoro(1-3C)alkyl, difluoro(1-3C)alkyl trifluoro(1-3C)alkyl, (3-6C)cycloalkyl, cyclopropylamino, cyclopropylmethyl, (1-6C)alkyl, or a 5-membered heteroaryl having 1-2 ring heteroatoms independently selected from N, O and S. In one embodiment, the substituent is coupled to the nitrogen atom of the 5-membered azacyclic ring.

In one embodiment, R⁴ and R⁵ together with the carbon atom to which they are attached form a 5-membered azacyclic ring substituted with —SO₂R^{c}, where R^{c} is fluoro(1-3C)alkyl, difluoro(1-3C)alkyl trifluoro(1-3C)alkyl, or (3-6C)cycloalkyl. In one embodiment, the substituent is coupled to the nitrogen atom of the 5-membered azacyclic ring. In one embodiment, R⁴ and R⁵ together with the carbon atom to which they are attached form a 5-membered azacyclic ring substituted with —SO₂CH₃, —SO₂CH₂CH₃, —SO₂CH₂CH₂CH₃, —SO₂CH(CH₃)₂, —SO₂CH₂CH₂CF₃, —SO₂CF₃, —SO₂CF₂CF₃, SO₂CF₂H or —SO₂-cyclopropyl. In one embodiment, R⁴ and R⁵ together with the carbon atom to which they are attached form a 5-membered azacyclic ring substituted with —SO₂CF₃, SO₂CF₂H or —SO₂-cyclopropyl. In one embodiment, the substituent is coupled to the nitrogen atom of the 5-membered azacyclic ring.

In one embodiment, R⁴ and R⁵ together with the carbon atom to which they are attached form a 5-membered azacyclic ring substituted with a substituent selected from fluoromethyl, 3-fluoropropyl, 2-fluoroethyl, 2,2-difluoroethyl, 1,3-difluoroprop-2-yl, 2,2,2-trifluoroethyl, 3,3,3-trifluoropropyl, SO₂CH₃, SO₂CH₂CH₃, SO₂CH₂CH₂CH₃, SO₂CH(CH₃)₂, SO₂CH₂CH₂CF₃, SO₂CF₃, SO₂CF₂CF₃, SO₂CF₂H and —SO₂cyclopropyl. In one embodiment, the substituent is coupled to the nitrogen atom of the 5-membered azacyclic ring.

In one embodiment, R⁴ and R⁵ together with the carbon atom to which they are attached form a 3-6-membered carbocyclic ring optionally substituted with one or more substitutents independently selected from methyl and halogen. In one embodiment, R⁴ and R⁵ together with the carbon atom to which they are attached form a cyclopentyl ring. In one embodiment, R⁴ and R⁵ together with the carbon atom to which they are attached form a cyclobutyl ring.

In one embodiment, R⁴ and R⁵ together with the carbon atom to which they are attached form a 5-membered azacyclic ring substituted with (1-6C)alkyl. In one embodiment, R⁴ and R⁵ together with the carbon atom to which they are attached form a 4-membered azacyclic ring substituted with ethyl. In one embodiment, the substituent is coupled to the nitrogen atom of the 5-membered azacyclic ring.

In one embodiment, R⁴ and R⁵ together with the carbon atom to which they are attached form a 5-membered azacyclic ring substituted with (1-6Calkyl)C(═O)—. In one embodiment, R⁴ and R⁵ together with the carbon atom to which they are attached form a 5-membered azacyclic ring substituted with CH₃C(═O)—. In one embodiment, the substituent is coupled to the nitrogen atom of the 5-membered azacyclic ring.

In one embodiment, R⁴ and R⁵ together with the carbon atom to which they are attached form a 5-membered azacyclic ring substituted with phenylC(═O)—. In one embodiment, the substituent is coupled to the nitrogen atom of the 5-membered azacyclic ring.

In one embodiment, R⁴ and R⁵ together with the carbon atom to which they are attached form a 5-membered azacyclic ring substituted with cyclopropyl-C(═O)—. In one embodiment, the substituent is coupled to the nitrogen atom of the 5-membered azacyclic ring.

In one embodiment, R⁴ and R⁵ together with the carbon atom to which they are attached form a 5-membered azacyclic ring substituted with (1-6C alkyl)NHC(═O)—. In one embodiment, R⁴ and R⁵ together with the carbon atom to which they are attached form a 5-membered azacyclic ring substituted with CH₃CH₂NHC(═O)—. In one embodiment, the substituent is coupled to the nitrogen atom of the 5-membered azacyclic ring.

In one embodiment, R⁴ and R⁵ together with the carbon atom to which they are attached form a 5-membered azacyclic ring substituted with di(1-6C alkyl)NC(═O)—. In one embodiment, R⁴ and R⁵ together with the carbon atom to which they are attached form a 5-membered azacyclic ring substituted with Me₂NC(═O). In one embodiment, the substituent is coupled to the nitrogen atom of the 5-membered azacyclic ring.

In one embodiment, R⁴ and R⁵ together with the carbon atom to which they are attached form a 5-membered azacyclic ring substituted with cyano(1-6Calkyl). In one embodiment, R⁴ and R⁵ together with the carbon atom to which they are attached form a 5-membered azacyclic ring substituted with CNCH₂—. In one embodiment, the substituent is coupled to the nitrogen atom of the 5-membered azacyclic ring.

In one embodiment, R⁶ is hydrogen.

In one embodiment, R⁶ is (1-6C)alkyl. Examples include methyl, ethyl, propyl, isopropyl, butyl, isobutyl, sec-butyl, pentyl and hexyl. In one embodiment, R⁶ is methyl or ethyl.

In one embodiment, R⁶ is (2-6C)alkenyl. Examples include ethenyl, propenyl and butenyl. In one embodiment, R⁶ is 1-propen-3-yl. In one embodiment, R⁶ is propen-1-yl, propen-2-yl or 2-buten-1-yl.

In one embodiment, R⁶ is (2-6C)alkynyl. In one embodiment, R⁶ is 1-propyn-3-yl or butyn-2-yl.

In one embodiment, R⁶ is (3-6C)cycloalkyl. In one embodiment, R⁶ is cyclopropyl.

In one embodiment, R⁶ is fluoro(1-6C)alkyl. In one embodiment, R⁶ is 2-fluoroethyl or 3-fluoropropyl.

In one embodiment, R⁶ is difluoro(1-6C)alkyl. In one embodiment, R⁶ is 2,2-difluoroethyl or 3,3-difluoropropyl.

In one embodiment, R⁶ is trifluoro(1-6C)alkyl. In one embodiment, R⁶ is 2,2,2-trifluoroethyl or 3,3,3-trifluoropropyl.

In one embodiment, R⁶ is (3-6C cycloalkyl)(1-3C)alkyl. In one embodiment, R⁶ is cyclopropylmethyl.

In one embodiment, R⁶ is hydroxy(1-6C)alkyl. In one embodiment, R⁶ is hydroxymethyl, 2-hydroxyethyl, 2-hydroxyprop-1-yl, 2-hydroxyprop-2-yl or 3-hydroxyprop-1-yl.

In one embodiment, R⁶ is (1-3C alkoxy)(1-6C)alkyl. In one embodiment, R⁶ is 2-methoxyethyl or 2-ethoxyethyl.

In one embodiment, R⁶ is (1-3C alkylsufanyl)(1-3C)alkyl. In one embodiment, R⁶ is 2-(methylsulfanyl)ethyl(MeS—CH₂CH₂—).

In one embodiment, R⁶ is (1-3C alkyl)OC(═O)(1-3C)alkyl. In one embodiment, R⁶ is CH₃CH₂OC(═O)CH₂— or CH₃OCH(═O)CH₂—.

In one embodiment, R⁶ is carboxy(1-6C)alkyl. In one embodiment, R⁶ is HOC(═O)CH₂—.

In one embodiment, R⁶ is fluoro(2-6C)alkenyl. In one embodiment, R⁶ is 3-fluoropropyn-2-yl.

In one embodiment, R⁶ is difluoro(2-c)alkenyl. In one embodiment, R⁶ is 3,3-difluoropropyn-2-yl.

In one embodiment, R⁶ is (1-6C)alkylC(═O)CH₂—. In one embodiment, R⁶ is CH₃C(═O)CH₂—.

In one embodiment, R⁶ is (1-6C)alkyl, (2-6C)alkenyl, (2-6C)alkynyl or (3-6C)cycloalkyl.

In one embodiment, R⁶ is fluoro(1-6C)alkyl, difluoro(1-6C)alkyl or trifluoro(1-6C)alkyl.

In one embodiment, R⁶ is hydroxy(1-6C)alkyl, (1-3C alkoxy)(1-6C)alkyl, (1-3C alkylsufanyl)(1-3C)alkyl, (1-3C alkyl)C(═O)O(1-3C)alkyl or carboxy(1-6C)alkyl.

In one embodiment, R⁶ is selected from methyl, ethyl, 1-propen-3-yl, 1-propyn-3-yl, cyclopropyl, 2-fluoroethyl or 3-fluoropropyl, 2,2-difluoroethyl, 3,3-difluoropropyl, 2,2,2-trifluoroethyl, 3,3,3-trifluoropropyl, cyclopropylmethyl, hydroxymethyl, 2-hydroxyethyl, 2-methoxyethyl, 2-methylsulfanylethyl, CH₃CH₂C(═O)CH₂— and HOC(═O)CH₂—.

Particular examples of the residue at the 5-position of the imidazo[1,2-c]pyrimidine ring of Formula I include the structures:

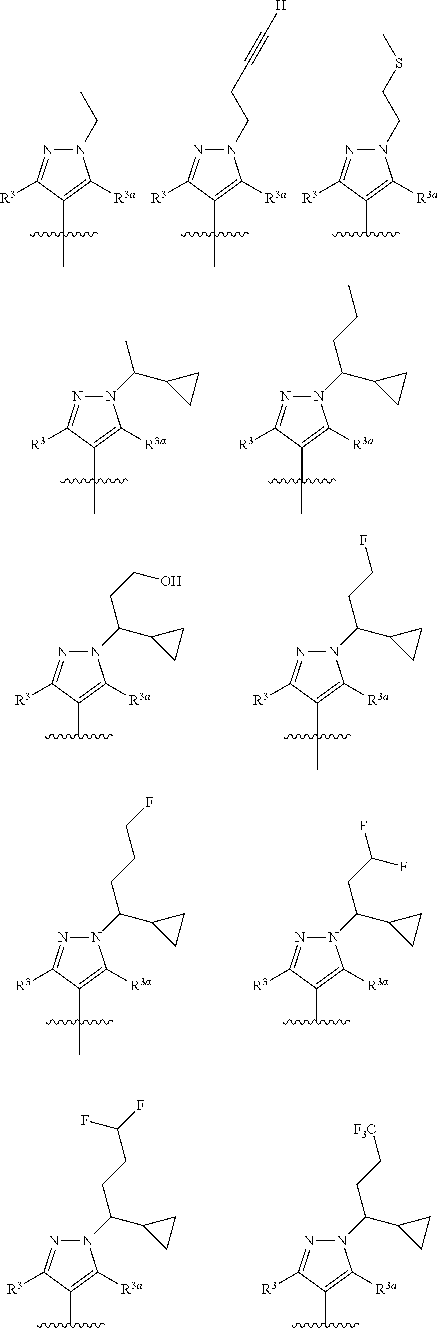

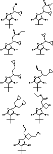

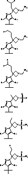

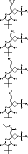

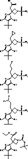

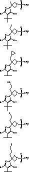

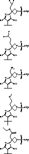

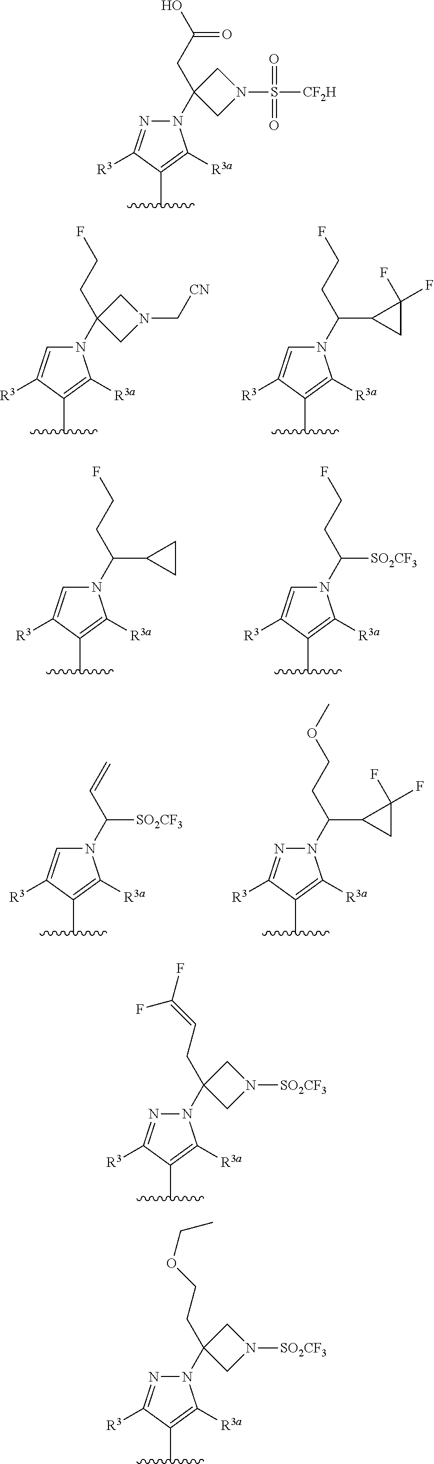

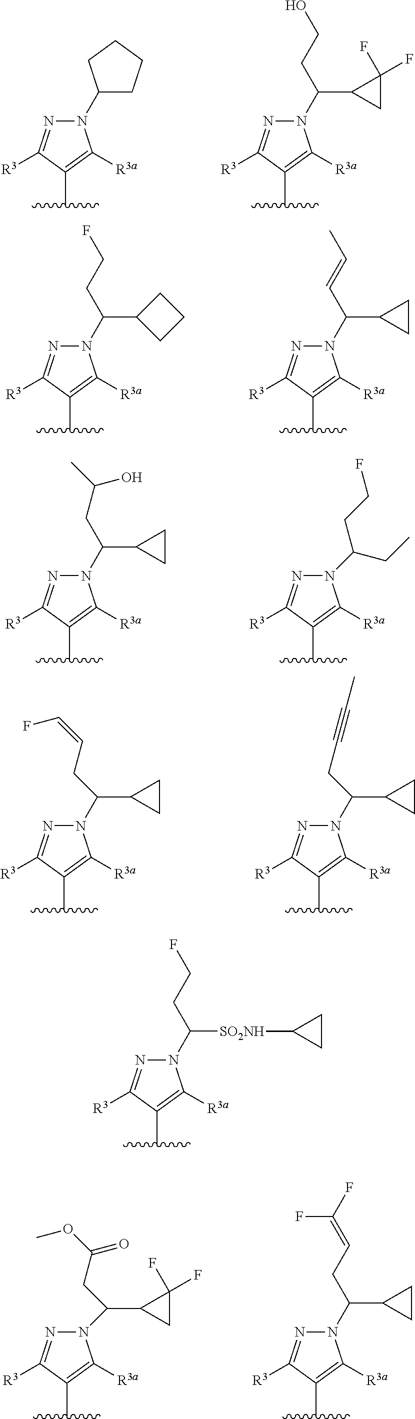

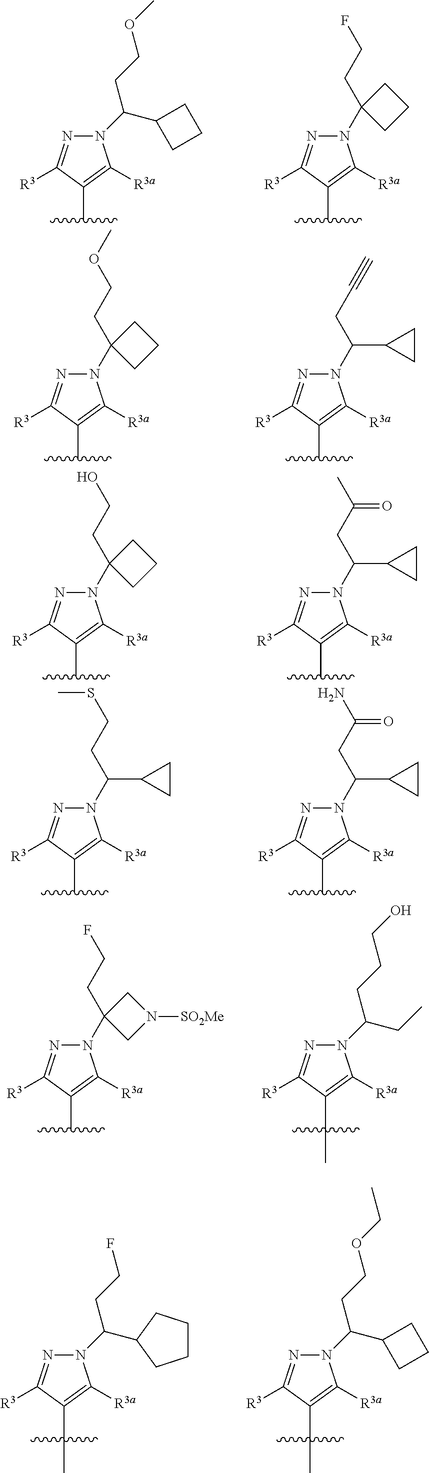

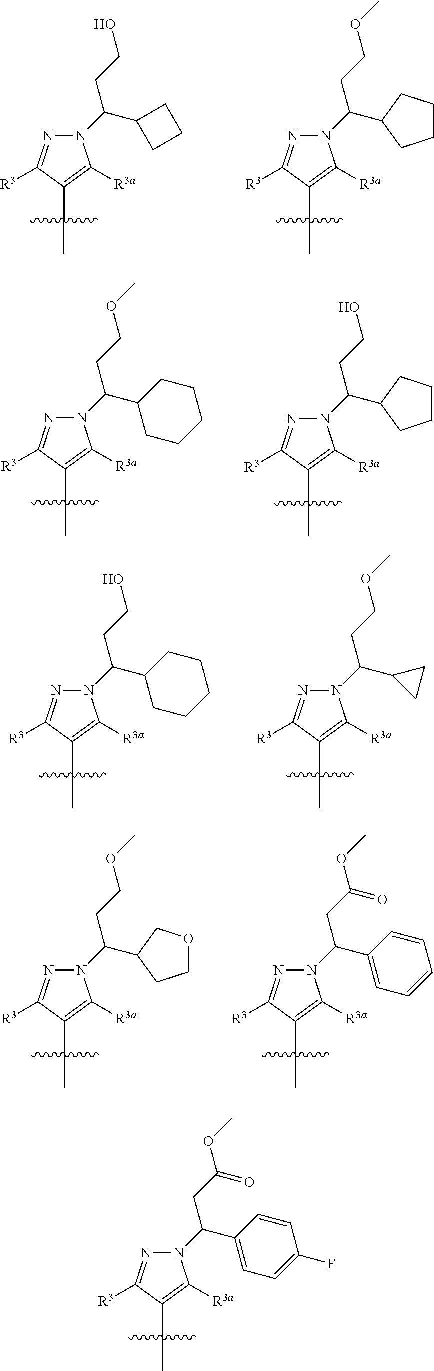

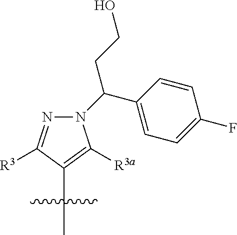

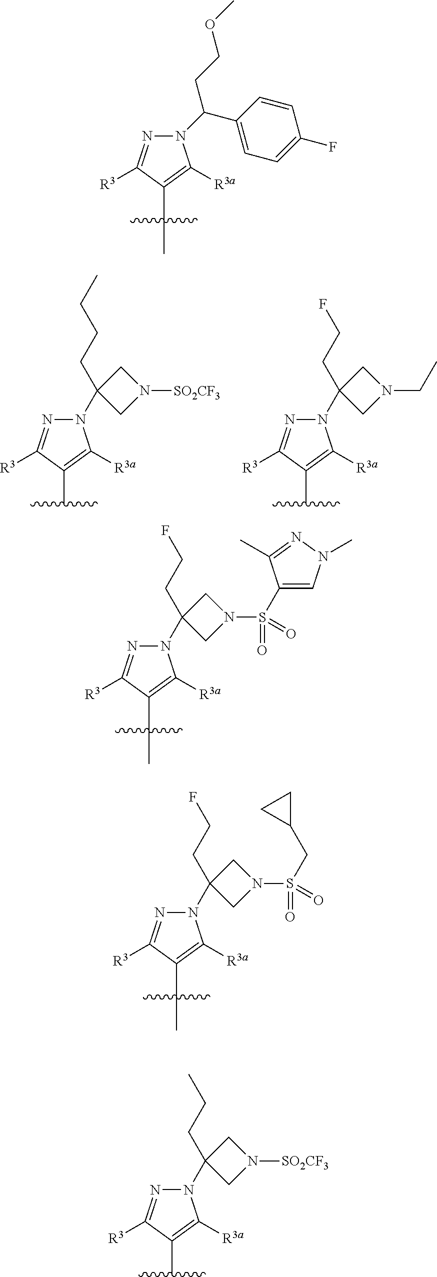

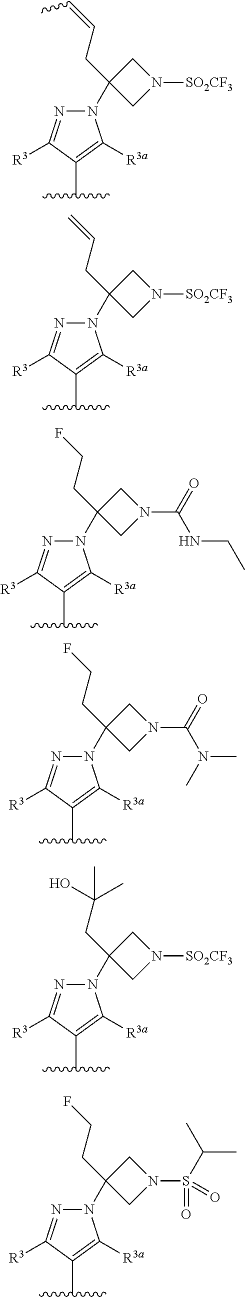

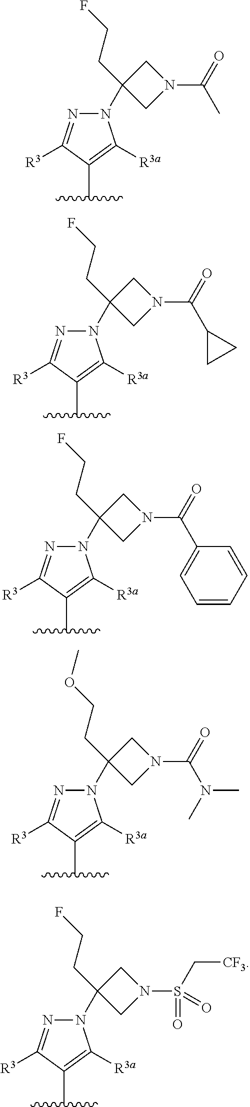

including enantiomers thereof, where the wavy line indicates the point of attachment to the imidazo[1,2-c]pyrimidine ring of Formula I. In certain embodiments of the above residues, R³ and R^{3a} are hydrogen.

In one embodiment of Formula I, R¹ is selected from hetAr¹, hetAr², hetAr³, Ar¹ and Ar²; R² is hydrogen; R³ is hydrogen; R^{3a} is hydrogen; R⁴ is hydrogen; R⁵ is hydrogen, (3-6C)cycloalkyl (optionally substituted by one or more halogens), or (3-6C)cycloalkylCH₂— (optionally substituted by one or more halogens); and R⁶ is (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, hydroxy(1-6C)alkyl, (1-3C alkoxy)(1-6C)alkyl, (1-3C alkylsufanyl)(1-3C)alkyl, (1-3C alkyl)OC(═O)(1-3C)alkyl or carboxy(1-6C)alkyl.

In one embodiment of Formula I, R¹ is hetAr¹; R² is hydrogen; R³ is hydrogen; R^{3a} is hydrogen; R⁴ is hydrogen; R⁵ is hydrogen, (3-6C)cycloalkyl (optionally substituted by one or more halogens), or (3-6C)cycloalkylCH₂— (optionally substituted by one or more halogens); and R⁶ is (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, hydroxy(1-6C)alkyl, (1-3C alkoxy)(1-6C)alkyl, (1-3C alkylsufanyl)(1-3C)alkyl, (1-3C alkyl)OC(═O)(1-3C)alkyl or carboxy(1-6C)alkyl.

In one embodiment of Formula I, R¹ is selected from hetAr¹, hetAr², hetAr³, Ar¹ and Ar²; R² is hydrogen; R³ is hydrogen; R^{3a} is hydrogen; R⁴ and R⁵ together with the carbon atom to which they are attached form a 4-membered azacyclic ring substituted with a substituent selected from fluoro(1-6C)alkyl, difluoro(1-6C)alkyl, trifluoro(1-6C)alkyl, (1-6Calkyl)C(═O)O—, and —SO₂R^{c}; and R⁶ is (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, hydroxy(1-6C)alkyl, (1-3C alkoxy)(1-6C)alkyl, (1-3C alkylsufanyl)(1-3C)alkyl, (1-3C alkyl)OC(═O)(1-3C)alkyl or carboxy(1-6C)alkyl.

In one embodiment of Formula I, R¹ is hetAr¹; R² is hydrogen; R³ is hydrogen; R^{3a} is hydrogen; R⁴ and R⁵ together with the carbon atom to which they are attached form a 4-membered azacyclic ring substituted with a substituent selected from fluoro(1-6C)alkyl, difluoro(1-6C)alkyl, trifluoro(1-6C)alkyl, (1-6C alkyl)C(═O)O—, and —SO₂R^{c}; and R⁶ is (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, hydroxy(1-6C)alkyl, (1-3C alkoxy)(1-6C)alkyl, (1-3C alkylsufanyl)(1-3C)alkyl, (1-3C alkyl)OC(═O)(1-3C)alkyl or carboxy(1-6C)alkyl.

In one embodiment of Formula I, R¹ is pyrazol-4-yl, thiazol-5-yl, imidazol-1-yl or 1,3,4-thiadiazol-2-yl optionally substituted with one or more substituents independently selected from methyl, ethyl, isopropyl, isobutyl, 2,2,2-trifluoroethyl, (2-isopropoxy)ethyl, trimethylsilylethoxymethyl, cyclobutyl, oxetanyl, 4-tetrahydro-2H-pyranyl, (4-methylpiperazinyl)ethyl and pyrid-3-ylmethyl; R² is hydrogen; R³ and R^{3a} are hydrogen; R⁴ is hydrogen; R⁵ is hydrogen, (3-6C)cycloalkyl (optionally substituted by one or more halogens), or (3-6C)cycloalkylCH₂— (optionally substituted by one or more halogens); and R⁶ is (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, hydroxy(1-6C)alkyl, (1-3C alkoxy)(1-6C)alkyl, (1-3C alkylsufanyl)(1-3C)alkyl, (1-3C alkyl)OC(═O)(1-3C)alkyl or carboxy(1-6C)alkyl.

In one embodiment of Formula I, R¹ is pyrazol-4-yl, thiazol-5-yl, imidazol-1-yl or 1,3,4-thiadiazol-2-yl optionally substituted with one or more substituents independently selected from methyl, ethyl, isopropyl, isobutyl, 2,2,2-trifluoroethyl, (2-isopropoxy)ethyl, trimethylsilylethoxymethyl, cyclobutyl, oxetanyl, 4-tetrahydro-2H-pyranyl, (4-methylpiperazinyl)ethyl and pyrid-3-ylmethyl; R² is hydrogen; R³ and R^{3a} are hydrogen; R⁴ and R⁵ together with the carbon atom to which they are attached form a 4-membered azacyclic ring substituted with a substituent selected from fluoromethyl, 3-fluoropropyl, 2-fluoroethyl, 2,2-difluoroethyl, 1,3-difluoroprop-2-yl, 2,2,2-trifluoroethyl, and 3,3,3-trifluoropropyl; and R⁶ is (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, hydroxy(1-6C)alkyl, (1-3C alkoxy)(1-6C)alkyl, (1-3C alkylsufanyl)(1-3C)alkyl, (1-3C alkyl)OC(═O)(1-3C)alkyl or carboxy(1-6C)alkyl.

In one embodiment of Formula I, R¹ is pyrazol-4-yl optionally substituted with one or more substituents independently selected from (1-6C)alkyl, fluoro(1-6C)alkyl, difluoro(1-6C)alkyl, trifluoro(1-6C)alkyl, (1-4C alkoxy)(1-6C)alkyl, trimethylsilyl(1-4C alkoxy)(1-6C)alkyl, (3-6C)cycloalkyl, a 4-6 membered oxacyclic ring, hetCyc^{a}(1-2C)alkyl, hetAr^{a}(1-2C)alkyl and (1-4C alkylsulfonyl)(1-6C alkyl); R² is hydrogen; R³ and R^{3a} are hydrogen; R⁴ is hydrogen; R⁵ is hydrogen, (3-6C)cycloalkyl (optionally substituted by one or more halogens), or (3-6C)cycloalkylCH₂— (optionally substituted by one or more halogens); and R⁶ is (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, hydroxy(1-6C)alkyl, (1-3C alkoxy)(1-6C)alkyl, (1-3C alkylsufanyl)(1-3C)alkyl, (1-3C alkyl)OC(═O)(1-3C)alkyl or carboxy(1-6C)alkyl.

In one embodiment of Formula I, R¹ is pyrazol-4-yl optionally substituted with one or more substituents independently selected from (1-6C)alkyl, fluoro(1-6C)alkyl, difluoro(1-6C)alkyl, trifluoro(1-6C)alkyl, (1-4C alkoxy)(1-6C)alkyl, trimethylsilyl(1-4C alkoxy)(1-6C)alkyl, (3-6C)cycloalkyl, a 4-6 membered oxacyclic ring, hetCyc^{a}(1-2C)alkyl, hetAr^{a}(1-2C)alkyl and (1-4C alkylsulfonyl)(1-6C alkyl); R² is hydrogen; R³ and R^{3a} are hydrogen; R⁴ and R⁵ together with the carbon atom to which they are attached form a 4-membered azacyclic ring substituted with —SO₂CH₃, —SO₂CH₂CH₃, —SO₂CH₂CH₂CH₃, —SO₂CH(CH₃)₂, —SO₂CH₂CH₂CF₃, —SO₂CF₃, —SO₂CF₂CF₃ SO₂CF₂H or —SO₂-cyclopropyl; and R⁶ is (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, hydroxy(1-6C)alkyl, (1-3C alkoxy)(1-6C)alkyl, (1-3C alkylsufanyl)(1-3C)alkyl, (1-3C alkyl)OC(═O)(1-3C)alkyl or carboxy(1-6C)alkyl.

In one embodiment of Formula I, R¹ is pyrazol-4-yl optionally substituted with one or more substituents independently selected from methyl, ethyl, isopropyl, isobutyl, 2,2,2-trifluoroethyl, (2-isopropoxy)ethyl, trimethylsilylethoxymethyl, cyclobutyl, oxetanyl, 4-tetrahydro-2H-pyranyl, (4-methylpiperazinyl)ethyl and pyrid-3-ylmethyl; R² is hydrogen; R³ and R^{3a} are hydrogen; R⁴ is hydrogen; R⁵ is hydrogen, (3-6C)cycloalkyl (optionally substituted by one or more halogens), or (3-6C)cycloalkylCH₂— (optionally substituted by one or more halogens); and R⁶ is (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, hydroxy(1-6C)alkyl, (1-3C alkoxy)(1-6C)alkyl, (1-3C alkylsufanyl)(1-3C)alkyl, (1-3C alkyl)OC(═O)(1-3C)alkyl or carboxy(1-6C)alkyl.

In one embodiment of Formula I, R¹ is pyrazol-4-yl optionally substituted with one or more substituents independently selected from methyl, ethyl, isopropyl, isobutyl, 2,2,2-trifluoroethyl, (2-isopropoxy)ethyl, trimethylsilylethoxymethyl, cyclobutyl, oxetanyl, 4-tetrahydro-2H-pyranyl, (4-methylpiperazinyl)ethyl and pyrid-3-ylmethyl; R² is hydrogen; R³ and R^{3a} are hydrogen; R⁴ and R⁵ together with the carbon atom to which they are attached form a 4-membered azacyclic ring substituted with —SO₂CH₃, —SO₂CH₂CH₃, —SO₂CH₂CH₂CH₃, —SO₂CH(CH₃)₂, —SO₂CH₂CH₂CF₃, —SO₂CF₃, —SO₂CF₂CF₃, SO₂CF₂H or —SO₂-cyclopropyl; and R⁶ is (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, hydroxy(1-6C)alkyl, (1-3C alkoxy)(1-6C)alkyl, (1-3C alkylsufanyl)(1-3C)alkyl, (1-3C alkyl)OC(═O)(1-3C)alkyl or carboxy(1-6C)alkyl.

It will be appreciated that certain compounds according to the invention may contain one or more centers of asymmetry and may therefore be prepared and isolated as a mixture of isomers such as a racemic or diastereomeric mixture, or in an enantiomerically or diastereomerically pure form. It is intended that all stereoisomeric forms of the compounds of the invention, including but not limited to, diastereomers, enantiomers and atropisomers, as well as mixtures thereof such as racemic mixtures, form part of the present invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

When words are used to describe a substituent, the rightmost-described component of the substituent is the component that has the free valence. To illustrate, 2-methylsulfanyl)ethyl refers to an ethyl radical, wherein the radical is on the first carbon atom of the ethyl group and the second carbon atom of the ethyl radical is substituted with a methylsulfanyl group as shown:

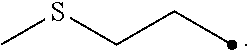

The term “(1-3C)alkyl”, “(1-4C)alkyl”, “(1-6C)alkyl” as used herein refers to saturated linear or branched-chain monovalent hydrocarbon radicals of one to three carbon atoms, one to four carbon atoms, or one to six carbon atoms, respectively. Examples include, but are not limited to, methyl, ethyl, 1-propyl, isopropyl, 1-butyl, isobutyl, sec-butyl, tert-butyl, 2-methyl-2-propyl, pentyl, and hexyl.

The terms “(1-4C)alkoxy” and “(1-6C)alkoxy”, as used herein refer to saturated linear or branched-chain monovalent alkoxy radicals of one to four carbon atoms or one to six carbon atoms, respectively, wherein the radical is on the oxygen atom. Examples include methoxy, ethoxy, propoxy, isopropoxy, and butoxy.

The term “fluoro(1-6C)alkyl” as use herein refers to saturated linear or branched-chain monovalent radicals of one to six carbon atoms, wherein one of the hydrogen atoms is replaced by fluorine. Examples include fluoromethyl, 3-fluoropropyl and 2-fluoroethyl.

The term “difluoro(1-6C)alkyl” as use herein refers to saturated linear or branched-chain monovalent radicals of one to six carbon atoms, wherein two of the hydrogen atoms are replaced by fluorine. Examples include difluoromethyl, 2,2-difluoroethyl, 3,3-difluoropropyl and 1,3-difluoroprop-2-yl.

The term “trifluoro(1-6C)alkyl” and “trifluoro(1-3C)alkyl” as use herein refers to saturated linear or branched-chain monovalent radicals of one to six carbon atoms and one to three carbon atoms, respectively, wherein three of the hydrogen atoms are replaced by fluorine. Examples include trifluoromethyl, 2,2,2-trifluoroethyl, and 3,3,3-trifluoropropyl.

The term “tetrafluoro(1-6C)alkyl” as used herein refers to saturated linear or branched-chain monovalent radicals of one to six carbon atoms, wherein four of the hydrogen atoms are replaced by fluorine. An example is 1,1,2,2-tetrafluoropropane.

The term “(1-4C alkoxy)(1-6C)alkyl” as used herein refers to saturated linear or branched-chain monovalent radicals of one to six carbon atoms, wherein one of the hydrogen atoms is replaced by a (1-4C alkoxy) group as defined herein. Examples include methoxymethyl (CH₃OCH₂—) and methoxyethyl (CH₃OCH₂CH₂—).

The term “trimethylsilyl(1-4C alkoxy)(1-6C)alkyl” as used herein refers to saturated linear or branched-chain monovalent radicals of one to six carbon atoms, wherein one of the hydrogen atoms is replaced by a trimethylsilyl(1-4C alkoxy) group. An example includes trimethylsilylethoxymethyl (Me₃SiCH₂CH₂OCH₂—).

The term “trimethylsilyl(1-4C alkoxy)” as used herein refers to saturated linear or branched-chain monovalent alkoxy radicals of one to four carbon atoms in which the radical is on the oxygen atom, wherein one of the hydrogen atoms is replaced by a trimethylsilyl group.

The term “(1-4C alkylsulfonyl)(1-6C alkyl)” as used herein refers to saturated linear or branched-chain monovalent radicals of one to six carbon atoms, wherein one of the hydrogen atoms is replaced by a (1-4C alkyl)sulfonyl group, that is, a (1-4C)SO₂— group.

The term “halogen” includes fluoro, chloro, bromo and iodo.

In instances where the term “heterocycle” is used, the term is intended to refer to a saturated or partially unsaturated heterocyclic ring. In one embodiment, the term “heterocycle” as used herein refers to a saturated heterocyclic ring.

It will also be appreciated that certain compounds of Formula I may be used as intermediates for the preparation of further compounds of Formula I.

The compounds of Formula I include salts thereof. In certain embodiments, the salts are pharmaceutically acceptable salts. In addition, the compounds of Formula I include other salts of such compounds which are not necessarily pharmaceutically acceptable salts, and which may be useful as intermediates for preparing and/or purifying compounds of Formula I and/or for separating enantiomers of compounds of Formula I.

The term “pharmaceutically acceptable” indicates that the substance or composition is compatible chemically and/or toxicologically, with the other ingredients comprising a formulation, and/or the mammal being treated therewith.

It will further be appreciated that the compounds of Formula I and their salts may be isolated in the form of solvates, and accordingly any such solvate is included within the scope of the present invention. the compounds of the present invention. For example, compounds of Formula I and their salts can exist in unsolvated as well as solvated forms with pharmaceutically acceptable solvents such as water, ethanol, and the like.

Compounds of the invention may also contain unnatural proportions of atomic isotopes at one or more of the atoms that constitute such compounds. That is, an atom, in particular when mentioned in relation to a compound according to Formula I, comprises all isotopes and isotopic mixtures of that atom, either naturally occurring or synthetically produced, either with natural abundance or in an isotopically enriched form. For example, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof; when nitrogen is mentioned, it is understood to refer to ¹³N, ¹⁴N, ¹⁵N or mixtures thereof; when oxygen is mentioned, it is understood to refer to ¹⁴O, ¹⁵O, ¹⁶O, ¹⁷O, ¹⁸O or mixtures thereof; and when fluoro is mentioned, it is understood to refer to ¹⁸F, ¹⁹F or mixtures thereof. The compounds according to the invention therefore also comprise compounds with one or more isotopes of one or more atom, and mixtures thereof, including radioactive compounds, wherein one or more non-radioactive atoms has been replaced by one of its radioactive enriched isotopes. Radiolabeled compounds are useful as therapeutics, research reagents, e.g., assay reagents, and diagnostic agents, e.g., in vivo imaging agents. All isotopic variations of the compounds of the present invention, whether radioactive or not, are intended to be encompassed within the scope of the present invention.

The compounds of this invention also include the compounds of Examples 1-102 described below, with the exception of the examples labeled as “reference examples”. Compounds labeled “Reference Examples” (i.e., Examples 75, 84, 92, 93, 96, 97, and 99) were found to be weakly active in the in vitro assays described below, and are provided to illustrate representative methodology in preparing compounds of Formula I. Accordingly, in one embodiment, the compounds of this invention include the compounds named in Examples 1-74, 76-83, 85-91, 94, 95, 98, and 100-102.

The present invention further provides a process for the preparation of a compound of Formula I or a pharmaceutically acceptable salt thereof as defined herein which comprises:

(a) for a compound of Formula I where R⁴ is hydrogen; R⁵ is hydrogen, (3-6C)cycloalkyl (optionally substituted by one or more halogens) or (3-6C)cycloalkylCH₂-(optionally substituted by one or more halogens); and R⁶ is (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, and R¹, R², R³, X¹ and X² are as defined for Formula I, reacting a corresponding compound of formula II

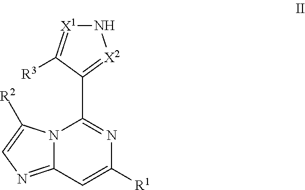

with a corresponding compound having the formula

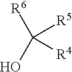

where R⁴ is hydrogen; R⁵ is hydrogen, (3-6C)cycloalkyl (optionally substituted by one or more halogens) or (3-6C)cycloalkylCH₂— (optionally substituted by one or more halogens); and R⁶ is (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, in the presence of triphenylphosphine and a coupling agent; or

(b) for a compound of Formula I where R⁶ is HOCH₂CH₂—; and R¹, R², R³, R⁴, R⁵, X¹ and X² are as defined for Formula I, treating a corresponding compound having the formula

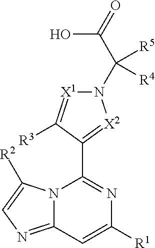

with a reducing agent; or

(c) for a compound of Formula I where R⁶ is methoxy(1-6C)alkyl; and R¹, R², R³, R⁴, R⁵, X¹ and X² are as defined for Formula I, treating a corresponding compound where R⁶ is hydroxy(1-6C)alkyl with methyl iodide in the presence of a base; or

(d) for a compound of Formula I where R⁶ is HOCH₂—; R⁵ is (3-6C)cycloalkyl; R⁴ is hydrogen; and R¹, R², R³, X¹ and X² are as defined for Formula I, reacting a compound of Formula II

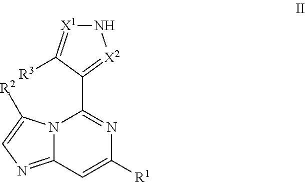

with a compound having the formula:

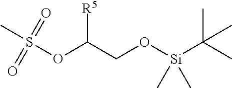

in the presence of a base; or

(e) for a compound Formula I where R⁶ is (1-3Calkyl)OC(═O)CH₂—; R⁵ is (3-6C)cycloalkyl; R⁴ is hydrogen; and R¹, R², R³, X¹ and X² are as defined for Formula I, reacting a compound of formula II

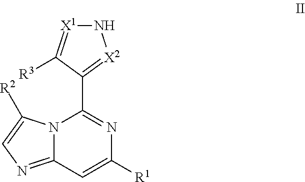

with a compound having the formula

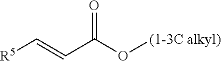

in the presence of 2,8,9-triisobutyl-2,5,8,9-tetraaza-1-phosphabicyclo[3.3.3]undecane; or

(f) for a compound of Formula I where R⁶ is fluoro(1-6C)alkyl; and R¹, R², R³, R⁴, R⁵, X¹ and X² are as defined for Formula I, reacting a corresponding compound of Formula I′

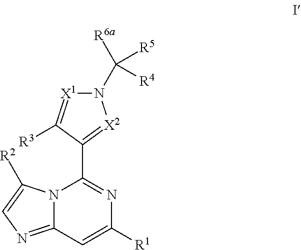

where R^{6a} is CH₃SO₃(1-6C)alkyl, and R¹, R², R³, R⁴, R⁵, X¹ and X² are as defined for Formula I, with tetrabutylammonium fluoride; or

(g) for a compound of Formula I wherein R⁴ and R⁵ form a 4-membered azacyclic ring substituted with fluoro(1-6C)alkyl, difluoro(1-6C)alkyl or trifluoro(1-6C)alkyl, and R¹, R², R³, R⁶, X¹ and X² are as defined for Formula I, coupling a corresponding compound having the formula III

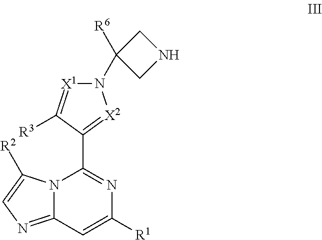

with a corresponding compound having the formula L³-R¹⁰, where L³ is a leaving group or atom and R¹⁰ is fluoro(1-6C)alkyl, difluoro(1-6C)alkyl or trifluoro(1-6C)alkyl, in the presence of a base; or

(h) for a compound of Formula I wherein R⁴ and R⁵ form a 4-membered azacyclic ring substituted with SO₂CF₃, and R¹, R², R³, R⁶, X¹ and X² are as defined for Formula I, reacting a corresponding compound having the formula III

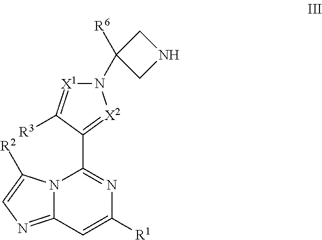

with trifluoromethanesulfonic anhydride in the presence of a base; or

(i) for a compound of Formula I wherein R⁴ and R⁵ form a 4-membered azacyclic ring substituted with SO₂R^{c}, wherein R^{c}, R¹, R², R³, R⁶, X¹ and X² are as defined for Formula I, coupling a corresponding compound having the formula III

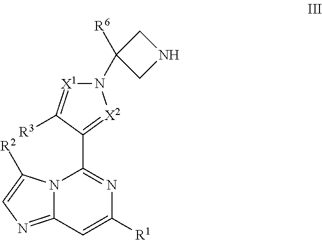

with a corresponding compound having the formula Cl—SO₂R^{c} in the presence of a base; or

(j) for a compound of Formula I wherein R² is Cl, and R¹, R³, R⁴, R⁵, R⁶, X¹ and X² are as defined for Formula I, reacting a corresponding compound of Formula I″

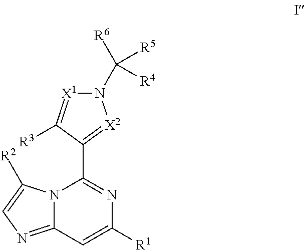

wherein R² is hydrogen, and R¹, R³, R⁴, R⁵, R⁶, X¹ and X² are as defined for Formula I, with 1-chloropyrrolidine-2,5-dione; or

(k) for a compound of Formula I wherein R² is CN, and R¹, R³, R⁴, R⁵, R⁶, X¹ and X² are as defined for Formula I, reacting a corresponding compound of Formula I″

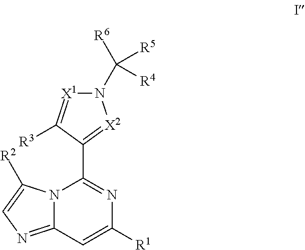

wherein R² is hydrogen, and R¹, R³, R⁴, R⁵, R⁶, X¹ and X² are as defined for Formula I, with 1-iodopyrrolidinine-2,5-dione followed by treatment of the resulting 3-iodo-substituted derivative of I′ with CuCN; or

(l) for a compound of Formula I wherein R² is F, and R¹, R³, R⁴, R⁵, R⁶, X¹ and X² are as defined for Formula I, reacting a corresponding compound of Formula I″

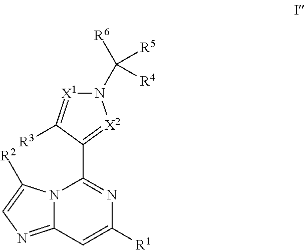

wherein R² is hydrogen, and R¹, R³, R⁴, R⁵, R⁶, X¹ and X² are as defined for Formula I, with an electrophilic fluorinating agent; or

(m) for a compound of Formula I wherein R² is F, and R¹, R³, R⁴, R⁵, R⁶, X¹ and X² are as defined for Formula I, reacting a corresponding compound of Formula I″′

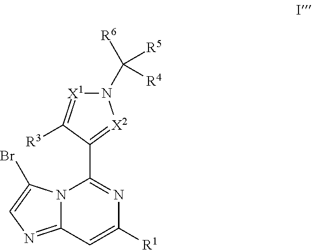

with an alkyl lithium or alkyl magnesium halide reagent, followed by treatment with an electrophilic fluorinating agent; and

optionally removing any protecting groups and optionally preparing a pharmaceutically acceptable salt thereof.

In one embodiment of any of the above recited methods, X¹ is N and X² is CR^{3a}.

Referring to method (a) suitable coupling agents include diisopropyl azodicarboxylate (DIAD) or to diethyl azodicarboxylate (DEAD). The reaction is preferably performed at elevated temperatures, for example at 60° C.

Referring to method (b), suitable reducing agents include LiBH₄, Na(OAc)₃BH and NaCNBH₃.

Referring to method (c), suitable bases include alkali metal hydrides such as NaH.

Referring to method (d), suitable bases include alkali metal hydrides such as NaH.

Referring to method (g), suitable bases include amine bases, such as DIEA (diisopropylethylamine) or triethylamine, or alkali metal carbonates such as for example cesium carbonate, sodium carbonate, potassium carbonate. Suitable solvents include dichloromethane, dichloroethane, THF, acetonitrile and DMF. The reaction is conveniently performed at temperatures between 0° C. and ambient temperature. The leaving atom L³ may be a halogen atom, for example chloro. Alternative, L³ may be a leaving group, such as a triflate (OTf) or sulfonyl chloride (SO₂Cl).

Referring to methods (h) and (i), suitable bases include amine bases, such as DIEA or triethylamine. Suitable solvents include neutral solvents such as dichloromethane and dichloroethane. The reaction is conveniently performed at temperatures between 0° C. and ambient temperature.

Referring to method (j) suitable solvents include dichloromethane and dichloroethane. The reaction is conveniently performed at temperatures between 0° C. and ambient temperature.

Referring to method (k), suitable solvents for the reaction with 1-iodopyrrolidine-2,5-dione include dichloromethane and dichloroethane. The reaction is conveniently performed at temperatures between 0° C. and ambient temperature. A suitable solvent for the reaction of the iodo intermediate with CuCN is DMF.

Referring to methods (l) and (m), an example of an electrophilic fluorinating agent is 1-chloromethyl-4-fluoro-1,4-diazoniabicyclo[2.2.2]octane bis(tetrafluoroborate) (also known as Selectfluor). The reaction is conveniently performed at ambient temperature or at elevated temperatures in a suitable solvent such as acetonitrile for method (l) or an ether solvent for method (m).

Compounds of formula II can be prepared by coupling a corresponding compound having the formula IV

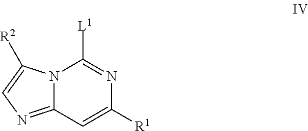

or a protected derivative thereof, where L¹ is a leaving atom and R¹ and R² are as defined for Formula I, with a corresponding compound having the formula V

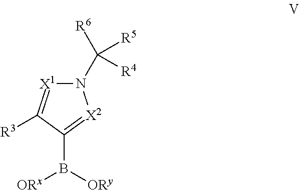

where R³, R⁴, R⁵, R⁶, X¹ and X² are as defined for Formula I and R^{x} and R^{y} are hydrogen or (1-6C)alkyl, or R^{x} and R^{y} together with the atoms to which they are connected form a 5-6 membered ring optionally substituted with 1-4 substituents selected from (1-3C alkyl), wherein said coupling takes place in the presence of a palladium catalyst and base and optionally in the presence of a ligand. In one embodiment, X¹ is N and X² is CR^{3a}. Suitable palladium catalysts include Pd(PPh₃)₄, Pd₂(dba)₃, Pd(OAc)₂, and Pd(PPh₃)₂Cl₂. Suitable ligands include XPHOS, DIPHOS or rac-BINAP. The base may be, for example, an alkali metal carbonate, hydroxide, alkoxide or acetate, such as for example cesium carbonate, sodium carbonate, potassium carbonate, sodium hydroxide, sodium tert-butoxide or potassium acetate. Convenient solvents include aprotic solvents such as ethers (for example tetrahydrofuran or p-dioxane), toluene, DMF or DME. The reaction can be conveniently performed at a temperature ranging from ambient temperature to 120° C., for example from 80 to 110° C. The leaving atom L¹ can be a halogen atom, such as chloride.

Alternatively, compounds of Formula I where R² is hydrogen can be prepared by cyclizing a corresponding compound having the formula VI

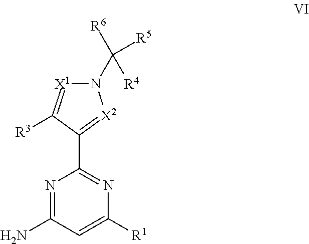

or a protected derivative thereof, where R¹, R³, R⁴, R⁵, R⁶, X¹ and X² are as defined for Formula I, with 2-chloroacetaldehyde in the presence of a base. In one embodiment, X¹ is N and X² is CR^{3a}. The base may be, for example, an alkali metal acetate, carbonate, hydroxide, or alkoxide, such as for example potassium acetate, cesium carbonate, sodium carbonate, potassium carbonate, sodium hydroxide or sodium tert-butoxide. Suitable solvents include alcoholic solvents such as ethanol. The reaction is conveniently performed in the presence of a pH 7 buffer, such as a phosphate buffer. The reaction is conveniently performed at elevated temperatures, such as 90-100° C.

Compounds of Formula III can be prepared by reacting a compound of Formula II

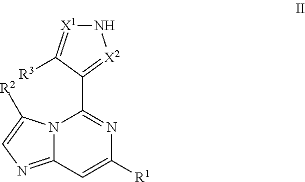

with a reagent having the formula

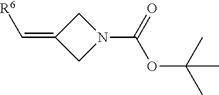

in the presence of DBU, followed by removal of the amine protecting group. In one embodiment of Formula II, X¹ is N and X² is CR^{3a}.

Amine groups in compounds described in any of the above methods may be protected with any convenient amine protecting group, for example as described in Greene & Wuts, eds., “Protecting Groups in Organic Synthesis”, 2^{nd} ed. New York; John Wiley & Sons, Inc., 1991. Examples of amine protecting groups include acyl and alkoxycarbonyl groups, such as t-butoxycarbonyl (BOC), and [2-(trimethylsilyl)ethoxy]methyl (SEM). Likewise, carboxyl groups may be protected with any convenient carboxyl protecting group, for 2^{nd} example as described in Greene & Wuts, eds., “Protecting Groups in Organic Synthesis”, ed. New York; John Wiley & Sons, Inc., 1991. Examples of carboxyl protecting groups include (1-6C)alkyl groups, such as methyl, ethyl and t-butyl. Alcohol groups may be protected with any convenient alcohol protecting group, for example as described in Greene & Wuts, eds., “Protecting Groups in Organic Synthesis”, 2^{nd} ed. New York; John Wiley & Sons, Inc., 1991. Examples of alcohol protecting groups include benzyl, trityl, silyl ethers, and the like.

The compounds of the formulas I′, I″, I″′, III, V and VI are also believed to be novel and are provided as further aspects of the invention.

The compounds of Formula I represent novel inhibitors of one or more JAK kinases. In particular, the compounds are inhibitors of Tyk2, JAK1, JAK2, and/or JAK3, and are useful in the treatment of cytokine or JAK kinase-associated diseases such as autoimmune diseases, inflammatory diseases, rejection of transplanted organs, tissues and cells, as well as hematologic disorders and malignancies and their co-morbidities.

The ability of compounds of the invention to act as inhibitors of Tyk2 may be demonstrated by the assay described in Example A.

The ability of compounds of the invention to act as inhibitors of JAK1 may be demonstrated by the assay described in Example B.

The ability of compounds of the invention to act as inhibitors of JAK2 may be demonstrated by the assay described in Example C

The ability of compounds of the invention to act as inhibitors of JAK3 may be demonstrated by the assay described in Example D.

Compounds of Formula I may be useful in the treatment of JAK kinase-associated diseases such as autoimmune diseases and inflammatory diseases.

Examples of autoimmune diseases and inflammatory diseases include, but are not limited to:

(i) arthritis, including rheumatoid arthritis, juvenile arthritis, psoriatic arthritis, reactive arthritis, ankylosing spondylitis, osteoarthritis, and seronegative arthopathies;

(ii) intestinal inflammations including Crohn's disease, ulcerative colitis, inflammatory bowel disease, celiac diseases, proctitis, and eosinophilic gastroenteritis;

(iii) airways diseases including asthma and other obstructive airway diseases, including severe refractory asthma, chronic asthma, airway hyper-responsiveness, bronchitis, allergic asthma, and chronic obstruction pulmonary disease;

(iv) allergic reactions including severe allergic reaction (including anaphylaxis);

(v) eye diseases, disorders or conditions including autoimmune diseases of the eye, uveitis including uveitis associated with Behcet's disease, lens-induced uveitis and optic neuritis;

(vi) skin diseases, conditions or disorders including psoriasis, atopic dermatitis, severe dermatitis, eczema, scleroderma, pruritus and other pruritic conditions, alopecia greata and mastocytosis;

(vii) sepsis, systemic inflammatory response syndrome, and neutropenic fever;

(viii) fibrosis, including hepatic fibrosis, idiopathic pulmonary fibrosis, myelofibrosis and scleroderma;

(ix) gout (resolution of tophi);

(x) lupus (also known as systemic lupus erythematosus), including manifestations such as cutaneous lupus, lupus nephritis, neuropsychiatric lupus and other manifestations;

(xi) neurodegenerative diseases including demyelinating diseases, such as multiple sclerosis, motor neuron disease, Alzheimer's disease, Parkinson's disease, amyotrophic lateral sclerosis, and ischemic reperfusion injury in stroke;

(xii) diabetes, including Type I diabetes and complications from diabetes, metabolic syndrome and obesity, and

(xiii) axial spondyloarthorpathy (axial SpA).

Additional examples of autoimmune diseases and inflammatory diseases include nephropathy, sarcoidosis, pancreatitis, autoimmune thyroiditis, fibromyalgia, atherosclerosis, autoimmune hemolytic anemia, autoimmune atrophic gastritis of pernicious anemia, autoimmune encephalomyelitis, autoimmune orchitis, Goodpasture's disease, autoimmune myocarditis, autoimmune thrombocytopenia, sympathetic ophthalmia, myasthenia gravis, Graves' disease, primary biliary cirrhosis, chronic aggressive hepatitis, membranous glomerulopathy, Sjogren's syndrome, Reiter's syndrome, systemic sclerosis, polyarteritis nodosa, bullous pemphigoid, Cogan's syndrome, Wegener's granulomatosis, cystic fibrosis, mixed connective tissue disease, antiphospholipid syndrome, polymyositis, dermatomyositis, membranous nephritis, primary sclerosing cholangitis, severe chronic urticaria, giant cell arteritis, eosinophilic esophagitis, and eosinophilic gastritis.

Accordingly, this invention further provides a method of treating a disease or disorder selected from an autoimmune disease and an inflammatory disease in a mammal in need thereof, comprising administering to a mammal a therapeutically effective amount of at least one compound of Formula I or a pharmaceutically acceptable salt thereof.

In one embodiment, the autoimmune or inflammatory disease is selected from lupus, psoriasis, psoriatic arthritis, rheumatoid arthritis, multiple sclerosis and inflammatory bowel diseases.

Compounds of the present invention may also be useful for treating organ, tissue and cell transplants, including bone marrow transplant, and in the treatment of autoimmune and inflammatory diseases and of complications arising therefrom.

Accordingly, this invention further provides a method of treating organ, tissue or cell transplant rejection in a mammal in need thereof, comprising administering to a mammal a therapeutically effective amount of at least one compound of Formula I or a pharmaceutically acceptable salt thereof.

Compounds of the present invention may also be useful in treating certain malignancies, including solid tumors, skin cancer, and hematological malignancies such as lymphomas and leukemias, and further may be useful in treating the complications thereof, including sequelae of hematologic malignancies (for example, in the treatment of splenomegaly in myelofibrosis), as well as cachexia in patients with solid tumors.

Accordingly, this invention further provides a method of treating malignancies in a mammal, which comprises administering to said mammal a therapeutically effective amount of a compound of Formula I.

Compounds of Formula I may be administered alone as a sole therapy or can be administered in addition with one or more other substances and/or treatments that work by the same or a different mechanism of action. These agents may include but are not limited to cyclosporin A (e.g. Sandimmune® or Neoral®), rapamycin, FK-506 (tacrolimus), leflunomide, deoxyspergualin, mycophenolate (e.g. Cellcept®, azathioprine (e.g. Imuran®), daclizumab (e.g. Zenapax®), OKT3 (e.g. Orthocolone®.), AtGam, aspirin, acetaminophen, ibuprofen, naproxen, piroxicam, antiinflammatory steroids (e.g. prednisolone or dexamethasone), methotrexate, statins, anti-TNF agents (e.g., Enbrel® (etanercept) or Humira® (adalimumab)), Orencia® (abatacept), cyclophosphamide, mycophenolic acid, hydroxychloroquine, and metformin. These agents may be administered with one or more compounds of Formula I as part of the same or separate dosage forms, via the same or different routes of administration, and on the same or different administration schedules according to standard pharmaceutical practice known to one skilled in the art.

In one embodiment, provided herein is a pharmaceutical combination comprising an effective amount of: (a) at least one compound of Formula I; and (b) at least one other agent selected from cyclosporin A (e.g. Sandimmune® or Neoral®), rapamycin, FK-506 (tacrolimus), leflunomide, deoxyspergualin, mycophenolate (e.g. Cellcept®, azathioprine (e.g. Imuran®), daclizumab (e.g. Zenapax®), OKT3 (e.g. Orthocolone®.), AtGam, aspirin, acetaminophen, ibuprofen, naproxen, piroxicam, antiinflammatory steroids (e.g. prednisolone or dexamethasone), methotrexate, statins, anti-TNF agents (e.g., Enbrel® (etanercept) or Humira® (adalimumab)), Orencia® (abatacept), cyclophosphamide, mycophenolic acid, hydroxychloroquine, and metformin for use in the treatment of an autoimmune disease and inflammatory disease in a mammal, wherein components (a) and (b) of the combination are in separate dosage forms or in the same dosage form.

The term “pharmaceutical combination” as used herein means a product that results from the mixing or combining of more than one active ingredient and includes both fixed and non-fixed combinations of the active ingredients. The term “fixed combination” means that the active ingredients, e.g. (a) a compound of Formula I and (b) another agent, are both administered to a patient simultaneously in the form of a single entity or dosage. The term “non-fixed combination” means that the active ingredients, e.g., (a) a compound of Formula I and (b) another agent, are both administered to a patient as separate entities either simultaneously, concurrently or sequentially with no specific time limits, wherein such administration provides therapeutically effective levels of the two compounds in the body of the patient. For a non-fixed combination, the individual combination partners of the combination may be administered separately at different times during the course of therapy or concurrently in divided or single combination forms.

In the field of medical oncology it is normal practice to use a combination of different forms of treatment to treat each patient with cancer. In medical oncology the other component(s) of such conjoint treatment in addition to compositions of the present invention may be, for example, surgery, radiotherapy, chemotherapy, signal transduction inhibitors and/or monoclonal antibodies.

Accordingly, the compounds of Formula I may be administered in combination with one or more agents selected from mitotic inhibitors, alkylating agents, anti-metabolites, antisense DNA or RNA, intercalating antibiotics, growth factor inhibitors, signal transduction inhibitors, cell cycle inhibitors, enzyme inhibitors, retinoid receptor modulators, proteasome inhibitors, topoisomerase inhibitors, biological response modifiers, anti-hormones, angiogenesis inhibitors, cytostatic agents anti-androgens, targeted antibodies, HMG-CoA reductase inhibitors, and prenyl-protein transferase inhibitors. These agents may be administered with one or more compounds of Formula I as part of the same or separate dosage forms, via the same or different routes of administration, and on the same or different administration schedules according to standard pharmaceutical practice known to one skilled in the art.

As used herein, the terms “treatment” or “treating” mean an alleviation, in whole or in part, of symptoms associated with a disorder or condition (e.g., autoimmune diseases, inflammatory diseases, rejection of transplanted organs, tissues and cells, as well as hematologic disorders and malignancies and their co-morbidities as described herein), or slowing, or halting of further progression or worsening of those symptoms.

The terms “effective amount” and “therapeutically effective amount” refer to an amount of compound that, when administered to a mammal in need of such treatment, is sufficient to (i) treat a particular disease, condition, or disorder, (ii) attenuate, ameliorate, or eliminate one or more symptoms of the particular disease, condition, or disorder, or (iii) delay the onset of one or more symptoms of the particular disease, condition, or disorder described herein. The amount of a compound of Formula I that will correspond to such an amount will vary depending upon factors such as the particular compound, disease condition and its severity, the identity (e.g., weight) of the mammal in need of treatment, but can nevertheless be routinely determined by one skilled in the art.

As used herein, the term “mammal” refers to a warm-blooded animal that has or is at risk of developing a disease described herein and includes, but is not limited to, guinea pigs, dogs, cats, rats, mice, hamsters, and primates, including humans.

Compounds of the invention may be administered by any convenient route, e.g. into the gastrointestinal tract (e.g. rectally or orally), the nose, lungs, musculature or vasculature, or transdermally or dermally. Compounds may be administered in any convenient administrative form, e.g. tablets, powders, capsules, solutions, dispersions, suspensions, syrups, sprays, suppositories, gels, emulsions, patches etc. Such compositions may contain components conventional in pharmaceutical preparations, e.g. diluents, carriers, pH modifiers, sweeteners, bulking agents, and further active agents. If parenteral administration is desired, the compositions will be sterile and in a solution or suspension form suitable for injection or infusion. Such compositions form a further aspect of the invention.

The present invention further provides a pharmaceutical composition, which comprises a compound of Formula I or a pharmaceutically acceptable salt thereof, as defined hereinabove, and a pharmaceutically acceptable carrier, diluent or excipient.

An example of a suitable oral dosage form is a tablet containing about 25 mg, 50 mg, 100 mg, 250 mg, or 500 mg of the compound of the invention compounded with about 90-30 mg anhydrous lactose, about 5-40 mg sodium croscarmellose, about 5-30 mg polyvinylpyrrolidone (“PVP”) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An aerosol formulation can be prepared by dissolving the compound, for example 5-400 mg, of the invention in a suitable buffer solution, e.g. a phosphate buffer, adding a tonicifier, e.g., a salt such sodium chloride, if desired. The solution is typically filtered, e.g., using a 0.2 micron filter, to remove impurities and contaminants.

The present invention further provides a compound of Formula I or a pharmaceutically acceptable salt thereof, for use in therapy. In one embodiment, the invention provides a compound of Formula I or a pharmaceutically acceptable salt thereof, for use in the treatment of cytokine or JAK kinase-associated diseases in a mammal.

In one embodiment, the invention provides a compound of Formula I or a pharmaceutically acceptable salt thereof, for use in the treatment of autoimmune diseases and inflammatory diseases in a mammal.

In one embodiment, the invention provides a compound of Formula I or a pharmaceutically acceptable salt thereof, for use in the treatment of transplant rejection in a mammal.

In one embodiment, the invention provides a compound of Formula I or a pharmaceutically acceptable salt thereof, for use in the treatment of hematologic disorders and malignancies in a mammal.

According to a further aspect, the present invention provides the use of a compound of Formula I or a pharmaceutically acceptable salt thereof, in the treatment of cytokine or JAK kinase-associated diseases in a mammal.

In one embodiment, the invention provides the use of a compound of Formula I or a pharmaceutically acceptable salt thereof, in the treatment of autoimmune diseases and inflammatory diseases.

In one embodiment, the invention provides the use of a compound of Formula I or a pharmaceutically acceptable salt thereof, in the treatment of organ, tissue or cell transplant rejection in a mammal.

In one embodiment, the invention provides the use of a compound of Formula I or a pharmaceutically acceptable salt thereof, in the treatment of malignancies in a mammal.

### EXAMPLES

The following examples illustrate the invention. In the examples described below, unless otherwise indicated all temperatures are set forth in degrees Celsius. Reagents were purchased from commercial suppliers such as Aldrich Chemical Company, Lancaster, Alfa, Aesar, TCI, Maybridge, or other suitable suppliers, and were used without further purification unless otherwise indicated. THF, DCM, toluene, DMF) and dioxane were purchased from Aldrich in Sure/Seal™ bottles and used as received.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried or dried under a stream of dry nitrogen.

Column chromatography was done on a Biotage system (Manufacturer: Dyax Corporation) having a silica gel or C-18 reverse phase column, or on a silica SepPak cartridge (Waters), or using conventional flash column chromatography on silica gel, unless otherwise specified.

### General Enzyme Inhibition Assay Method

The assays described in Examples A, B, C and D for the determination of Tyk2, JAK1, JAK2 and JAK3 kinase activity, respectively, utilized the Omnia® Kinase fluorescence peptide substrate-based technology (Invitrogen). The specific components of the assay mixture are described in Examples A, B, C and D. In these assays, Mg²⁺ is chelated upon phosphorylation of the Omnia peptide by the kinase to form a bridge between the chelation-enhanced fluorophore Sox and the phosphate, resulting in an increase in fluorescence emission at 485 nM when excited at 360 nM. The reactions were therefore read at excitation 360 nm and emission was measured at 485 nm every 50 seconds for 45 minutes using a PerkinElmer EnVision Multilabel Plate Reader.

The final buffer conditions for Tyk2, JAK1, JAK2, and JAK3 assays were as follows: 25 mM HEPES, pH 7.4, 10 mM MgCl₂, 0.01% Triton X-100 and 1 mM DTT.

### IC₅₀ Determinations

Compounds were prepared at 50× the final concentration in DMSO by conducting 3-fold serial dilutions from a 500-μM intermediate dilution to give a 10-point dosing curve having a high dose of 10 μM. Two-μL aliquots of these were transferred to a fresh plate for a ten-fold intermediate dilution with assay buffer. Five-μL aliquots of the diluted compounds were then transferred to 20-μL of assay mixtures described in Examples A, B, C and D for a final concentration of DMSO of 2%. A standard or reference compound was typically included on each assay plate to validate that plate. For each plate, percent of control (POC) values were calculated for each well according to the following equation:

$$POC = \frac{Sample - {\overline{X}}_{min}}{{\overline{X}}_{max} - {\overline{X}}_{min}} \times 100 ,$$

where X̅max=Average Uninhibited Controls

X̅min=Average Background

IC₅₀'s were estimated from the POC's using a standard 4-parameter logistic model:

$$Y = A + \frac{B - A}{1 + {( \frac{C}{X} )}^{D}} ,$$

where A=Minimum Y (Bottom Asymptote)

B=Maximum Y (Top Asymptote)

C=EC₅₀

D=Slope Factor

X=Compound Concentration (nM)

Y=POC

The IC₅₀ is defined as the concentration of inhibitor at which the POC equals 50 for the fitted curve.

### Example A

### Tyk2 Inhibition Assay

Compounds of Formula I were screened for their ability to inhibit Tyk2 using the general enzyme inhibition assay method, in which the assay mixture contained 10 μM (Km app) or 1 mM ATP, 8 μM Omnia® Y12 peptide (Catalog #IVGN KPZ3121C; Invitrogen Corporation, Carlsbad, Calif.) and 2 nM Tyk2 in a total volume of 20 μL. Human Tyk2 kinase domain, comprising amino acids 886 to 1187 with 10 additional histidine residues (histidine tag) on the carboxy terminus, was expressed and purified from bacculovirus in-house at Array BioPharma Inc. (Boulder, Colo.). The histidine tag was cleaved after purification using standard conditions.

### Example B

### JAK1 Inhibition Assay

Compounds of Formula I were screened for their ability to inhibit JAK1 using the general enzyme inhibition assay method, in which the assay mixture contained 40 μM (Km app) or 1 mM ATP, 8 μM Omnia® Y12 peptide (Catalog #IVGN KPZ3121C; Invitrogen Corporation, Carlsbad, Calif.) and 15 nM JAK1 in a total volume of 20 μL. JAK1 was purchased from Invitrogen Corporation, Carlsbad, Calif. (catalog #IVGN PV4775).

### Example C

### JAK2 Inhibition Assay

Compounds of Formula I were screened for their ability to inhibit JAK2 using the general enzyme inhibition assay method, in which the assay mixture contained 25 μM (Km app) or 1 mM ATP, 10 μM Omnia® Y7 peptide (Catalog #IVGN KNZ3071C, Invitrogen Corporation, Carlsbad, Calif.) and 5 nM JAK2 in a total volume of 20 μL. JAK2 was purchased from Invitrogen Corporation, Carlsbad, Calif. (catalog #IVGN PV4288).

### Example D

### JAK3 Inhibition Assay

Compounds of Formula I were screened for their ability to inhibit JAK3 using the general enzyme inhibition assay method, in which the assay mixture contained 10 μM (Km app) or 1 mM ATP, 10 μM Omnia® Y7 peptide (Catalog #IVGN KNZ3071C, Invitrogen Corporation, Carlsbad, Calif.) and 2.5 nM JAK3 in a total volume of 20 μL. JAK3 was purchased from Invitrogen Corporation, Carlsbad, Calif. (catalog #IVGN PV4080).

Compounds of Formula I are inhibitors of Tyk2, JAK1, JAK2 and/or JAK3. A compound is considered to be an inhibitor of Tyk2, JAK1, JAK2 and/or JAK3 if it has an IC₅₀ value equal to or less than 1000 nM when tested in the above assay of Example A, B, C or D, respectively.

Table A provides averaged IC₅₀ ranges for compounds described in the Examples when tested in the assays described in Examples A, B, C and D. For each IC₅₀ value shown in Table A, “A” represents an IC₅₀ value of less than 10 nM, “B” represents an IC₅₀ value of between 10 nM and 100 nM, “C” represents an IC₅₀ value of greater than 100 nM and less than 1000 nM, and “D” represents an IC₅₀ value of greater than 1000 nM.

TABLE A

| | Tyk2 IC₅₀ | JAK1 IC₅₀ | JAK2 IC₅₀ | |
|---|---|---|---|---|
| Example # | (nM) | (nM) | (nM) | JAK3 IC₅₀ (nM) |
| 1 | C | D | D | D |
| 2 | C | D | D | D |
| 3 | B | C | C | C |
| 4 | C | D | D | D |
| 5 | C | D | C | D |
| 6 | C | D | D | D |
| 7 | C | D | D | D |
| 8 | B | C | B | C |
| 9 | B | D | C | C |
| 10 | A | C | B | C |
| 11 | B | C | B | C |
| 12 | C | D | D | D |
| 13 | C | D | C | D |
| 14 | B | D | C | D |
| 15 | B | C | B | C |
| 16 | B | C | C | D |
| 17 | A | C | B | C |
| 18 | A | C | B | C |
| 19 | B | D | C | D |
| 20 | B | C | B | C |
| 21 | B | C | B | C |
| 22 | B | C | C | C |
| 23 | A | B | B | B |
| 24 | A | B | B | C |
| 25 | A | B | B | B |
| 26 | A | B | B | B |
| 27 | A | B | B | B |
| 28 | B | C | B | C |
| 29 | A | B | B | B |
| 30 | B | C | B | C |
| 31 | C | C | C | C |
| 32 | A | A | A | B |
| 33 | B | B | B | C |
| 34 | C | D | C | D |
| 35 | C | D | D | D |
| 36 | A | C | B | C |
| 37 | B | C | C | C |
| 38 | C | D | C | D |
| 39 | B | C | C | C |
| 40 | C | D | C | D |
| 41 | C | D | C | D |
| 42 | A | C | B | C |
| 43 | B | C | B | C |
| 44 | B | C | B | C |
| 45 | C | D | C | D |
| 46 | C | C | C | D |
| 47 | B | C | B | C |
| 48 | N/A | N/A | N/A | N/A |
| 49 | A | B | B | B |
| 50 | A | B | A | B |
| 51 | A | B | B | B |
| 52 | B | C | C | C |
| 53 | C | D | C | C |
| 54 | A | B | B | B |
| 55 | A | C | B | C |
| 56 | C | D | D | D |
| 57 | B | C | C | C |
| 58 | A | C | B | C |
| 59 | A | C | B | C |
| 60 | A | B | A | C |
| 61 | C | D | C | D |
| 62 | B | C | B | C |
| 63 | C | D | C | D |
| 64 | B | C | B | C |
| 65 | B | C | C | C |
| 66 | A | C | B | C |
| 67 | C | D | C | D |
| 68 | C | D | D | D |
| 69 | A | C | B | C |
| 70 | C | D | D | D |
| 71 | D | D | D | D |
| 72 | C | D | D | D |
| 73 | C | D | C | D |
| 74 | B | D | C | D |
| 75 | D | D | D | D |
| 76 | C | D | C | D |
| 77 | C | C | C | C |
| 78 | A | B | B | C |
| 79 | B | C | C | C |
| 80 | C | D | C | D |
| 81 | C | C | C | C |
| 82 | C | C | C | C |
| 83 | B | C | C | C |
| 84 | D | D | D | D |
| 85 | C | C | C | D |
| 86 | C | D | C | D |
| 87 | C | C | C | C |
| 88 | B | C | B | C |
| 89 | A | B | B | C |
| 90 | B | B | B | B |
| 91 | A | B | B | C |
| 92 | D | D | D | D |
| 93 | D | D | D | D |
| 94 | C | C | C | D |
| 95 | C | D | C | D |
| 96 | D | D | D | D |
| 97 | D | D | D | D |
| 98 | C | A | B | C |
| 99 | D | D | D | D |
| 100 | A | B | B | C |
| 101 | C | C | C | C |
| 102 | B | C | C | D |

N/A = not available

### Preparation A

### 5-chloro-7-(1-methyl-1H-pyrazol-4-yl)imidazo[1,2-c]pyrimidine

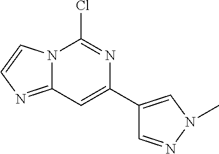 [SMILES: Cn1cc(-c2cc3nccn3c(Cl)n2)cn1]

To a suspension of 7-(1-methyl-1H-pyrazol-4-yl)imidazo[1,2-c]pyrimidin-5(6H)-one (9.60 g, 44.6 mmol) in dry DCM (90 mL) was added DIEA and the suspension stirred at ambient temperature for 5 minutes. The mixture was cooled to 0° C. and POCl₃ (12.3 mL, 134 mmol) was added over 5 minutes. The mixture was allowed to reach ambient temperature and the resulting thick slurry was treated with dry DCM (50 mL). The mixture was vigorously stirred at ambient temperature for 23 hours. The resulting light tan suspension was diluted with hexanes (90 mL) and collected by vacuum filtration. The collected solid was washed with Et₂O and dried in vacuum to give the crude product as a salt. The crude product was suspended in 5:20:75 MeOH/DIEA/EtOAc (200 mL) and stirred for 30 minutes at ambient temperature. The mixture was filtered through a SiO₂ plug capped with a layer of Celite®, eluting with 5% MeOH/EtOAc. The filtrate was concentrated and the residual solid was dried in a vacuum to provide the title compound (5.65 g, 54% yield) as a light cream colored solid. MS (apci) m/z=234.2 (M+H).

### Preparation B

### 7-(1-methyl-1H-pyrazol-4-yl)-5-(1H-pyrazol-4-yl)imidazo[1,2-c]pyrimidine (Method 1)

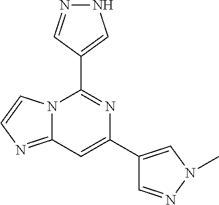 [SMILES: Cn1cc(-c2cc3nccn3c(-c3cn[nH]c3)n2)cn1]

To a mixture of 5-chloro-7-(1-methyl-1H-pyrazol-4-yl)imidazo[1,2-c]pyrimidine (132 mg, 0.565 mmol) and 4-(4,4,5,5-tetramethyl-1,3,2-dioxaborolan-2-yl)-1H-pyrazole (164 mg, 0.847 mmol) in DME (4 mL) was added 1M K₂CO₃ (1.69 mL, 1.69 mmol) and the resulting solution was purged with N₂ for 15 minutes. Pd(PPh₃)₄ (65.3 mg, 0.0565 mmol) was added, the flask was sealed, and the mixture was stirred at 90° C. for 15 hours. The reaction mixture was cooled to ambient temperature and diluted with H₂O (10 mL). The aqueous mixture was extracted with EtOAc, the extracts were combined and diluted with hexanes (1 vol). After standing for 15 minutes, the resulting precipitate was collected by vacuum filtration and washed with 50% EtOAc-hexanes to afford desired product. The EtOAc filtrate was extracted with 1M NaOH and the extracts were combined with the previous aqueous portion. The aqueous mixture was treated with 6M HCl to pH 4, and then with NaCl to saturation. The mixture was extracted with DCM and the combined extracts were dried over Na₂SO₄, filtered through a Celite® pad and concentrated. The residual product was combined with the previous batch and dried in a vacuum to provide the title compound (133 mg, 89% yield) as a light yellow solid. MS (apci) m/z=266.2 (M+H).

### Preparation C

### 7-(1-methyl-1H-pyrazol-4-yl)-5-(1H-pyrazol-4-yl)imidazo[1,2-c]pyrimidine hydrochloride (Method 2)

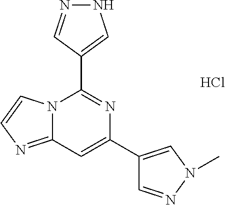 [SMILES: Cn1cc(-c2cc3nccn3c(-c3cn[nH]c3)n2)cn1 ; Cl]

### Step A: Preparation of 6-chloro-2-(1-((2-trimethylsilyl)ethoxy)methyl)-1H-pyrazol-4-yl)pyrimidin-4-amine and 2-chloro-6-(1-((2-trimethylsilyl)methyl)-1H-pyrazol-4-yl)pyrimidin-4-amine

2,6-Dichloropyrimidin-4-amine (4.00 g, 24.4 mmol), 4-(4,4,5,5-tetramethyl-1,3,2-dioxaborolan-2-yl)-1-(2-(trimethylsilyl)ethoxy)methyl)-1H-pyrazole (14.0 g, 36.6 mmol) and K₃PO₄ (15.5 g, 73.2 mmol) were suspended in dioxane (120 mL, 24.4 mmol) and H₂O (4.39 mL, 244 mmol). After degassing under nitrogen, Pd(PPh₃)₄ (1.41 g, 1.22 mmol) was added and the reaction sealed and stirred at 50° C. for 15 hours. After cooling, the reaction mixture was partitioned between saturated aqueous NaHCO₃ and EtOAc. The combined organic layers were washed with water and brine, dried with MgSO₄, filtered and concentrated under reduced pressure to afford the crude material as a thick yellow orange oil. The crude mixture was purified by silica chromatography, eluting with a 20-100% EtOAc/Hexanes gradient to afford 6-chloro-2-(1-((2-(trimethylsilyl)ethoxy)methyl)-1H-pyrazol-4-yl)pyrimidin-4-amine (4.00 g, 50.3%) and 2-chloro-6-(1-(((2-(trimethylsilyl)ethoxy)methyl)-1H-pyrazol-4-yl)pyrimidin-4-amine (2.96 g, 37.2% yield). MS (apci) m/z=326.1 (M+H). The structure and regioisomer of products were confirmed by observed nOe.

### Step B: Preparation of 6-(1-methyl-1H-pyrazol-4-yl)-2-(1-((2-(trimethylsilyl)ethoxy)-methyl)-1H-pyrazol-4-yl)pyrimidin-4-amine

6-Chloro-2-(1-((2-(trimethylsilyl)ethoxy)methyl)-1H-pyrazol-4-yl)pyrimidin-4-amine (1.00 g, 3.07 mmol), 1-methyl-4-(4,4,5,5-tetramethyl-1,3,2-dioxaborolan-2-yl)-1H-pyrazole (0.958 g, 4.60 mmol), K₃PO₄ (1.95 g, 9.21 mmol), and Pd(PPh₃)₄ (0.355 g, 0.307 mmol) were suspended in dioxane (15.3 mL) and H₂O (0.829 mL). After de-gassing with nitrogen, the reaction mixture was heated to 100° C. overnight. After cooling, the reaction mixture was diluted in EtOAc and washed with water and brine. The combined organic layers were dried with MgSO₄, filtered and concentrated down to an orange oil. Purification of the crude material by silica chromatography eluting with a gradient of 0-10% MeOH/EtOAc afforded 6-(1-methyl-1H-pyrazol-4-yl)-2-(1-((2-(trimethylsilyl)ethoxy) methyl)-1H-pyrazol-4-yl)pyrimidin-4-amine (0.623 g, 1.68 mmol, 54.7% yield) as a thick yellow oil. MS (apci) m/z=372.4 (M+H).

### Step C: Preparation of 7-(1-methyl-1H-pyrazol-4-yl)-5-(1-((2-(trimethylsilyl)ethoxy)-methyl)-1H-pyrazol-4-yl)imidazo[1,2-c]pyrimidine

6-(1-Methyl-1H-pyrazol-4-yl)-2-(1-((2-(trimethylsilyl)ethoxy)methyl)-1H-pyrazol-4-yl)pyrimidin-4-amine (2.0 g, 5.4 mmol) was suspended in a mixture of 40 mL of pH 7 phosphate buffer and 16 mL of EtOH. To the milky white mixture was added NaOAc (0.79 g, 9.7 mmol) followed by 2-chloroacetaldehyde (1.0 mL, 8.1 mmol). The reaction mixture was then heated to 95° C. After 5 hours, the reaction was incomplete and another portion of 2-chloroacetaldehyde (0.10 mL, 0.81 mmol) was added and the reaction was stirred for another 1 hour. After cooling, the reaction mixture was diluted with EtOAc and saturated NaHCO₃. After separation, the organic layer was washed with brine, dried with MgSO₄, filtered and concentrated in vacuo. The residue was diluted in diethyl ether, sonicated, and filtered to afford 0.88 g of 7-(1-methyl-1H-pyrazol-4-yl)-5-(1-((2-(trimethylsilyl)ethoxy)-methyl)-1H-pyrazol-4-yl)imidazo[1,2-c]pyrimidine as an off-white solid. Additional product was obtained by concentration of the filtrate and purification by silica chromatography using 0-10% MeOH/EtOAc. This afforded another 0.80 g of the intermediate. MS (apci) m/z=396.2 (M+H).

### Step D: Preparation of 7-(1-methyl-1H-pyrazol-4-yl)-5-(1H-pyrazol-4-yl)imidazo[1,2-c]pyrimidine hydrochloride

7-(1-Methyl-1H-pyrazol-4-yl)-5-(1-((2-(trimethylsilyl)ethoxy)methyl)-1H-pyrazol-4-yl)imidazo[1,2-c]pyrimidine (75 mg, 0.19 mmol) was dissolved in DCM (950 μL, 0.19 mmol). To this was added 4N HCl in dioxane (950 μL, 0.95 mmol) and stirred at ambient temperature for 1 hour, and the mixture was concentrated down to dryness to provide the title compound. MS (apci) m/z=266.2 (M+H).

### Preparation D

### Tert-butyl 3-(cyanomethyl)-3-(4-(4,4,5,5-tetramethyl-1,3,2-dioxaborolan-2-yl)-1H-pyrazol-1-yl)azetidine-1-carboxylate

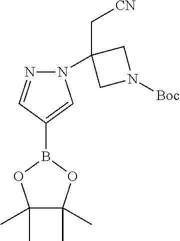 [SMILES: CC(C)(C)OC(=O)N1CC(CC#N)(n2cc(B3OC(C)(C)C(C)(C)O3)cn2)C1]

### Step A: Preparation of tert-butyl 3-(cyanomethylene)azetidine-1-carboxylate

In a 5 L flask, a suspension of NaH (24.531 g, 613.34 mmol) in 500 mL of THF was cooled in an ice bath. A solution of diethyl cyanomethylphosphonate (104.08 mL, 648.39 mmol) in THF (200 mL) was added dropwise. After addition, another 120 mL of THF was added to aid stirring. The reaction was warmed to ambient temperature for 1 hour then cooled back to 0° C. for 1 hour to give a milky yellow solution. Then a solution of tert-butyl 3-oxoazetidine-1-carboxylate (100.00 g, 584.13 mmol) in THF (400 mL) was added dropwise over an hour. The resultant reaction mixture was stirred for 15 hours, then quenched with water and concentrated to remove THF. The resultant aqueous solution was extracted with EtOAc. The combined organic layers were washed with brine and dried with MgSO₄. The filtrate was concentrated down to a yellow oil, which precipitated out a yellow solid after sitting overnight. This solid was diluted in cold EtOAc, sonicated, filtered and washed with cold EtOAc and hexanes to afford 82.09 g of a cream colored solid (80%). Additional product was isolated by concentrating the filtrate in vacuo and purifying by silica chromatography using a gradient of 20-30% EtOAc/Hexanes to afford an additional 18.6 g (18%) of tert-butyl 3-(cyanomethylene)azetidine-1-carboxylate. ¹H NMR (CDCl₃) δ 5.38 (m, 1H), 4.69-4.72 (m, 2H), 4.60-4.63 (m, 2H), 1.46 (s, 9H).

### Step B: Preparation of tert-butyl 3-(cyanomethyl)-3-(4-(4,4,5,5-tetramethyl-1,3,2-dioxaborolan-2-yl)-1H-pyrazol-1-yl)azetidine-1-carboxylate

In a 5 L flask, tert-butyl 3-(cyanomethylene)azetidine-1-carboxylate (Preparation F, Step A; 94.2 g, 485 mmol) and 4-(4,4,5,5-tetramethyl-1,3,2-dioxaborolan-2-yl)-1H-pyrazole (85.6 g, 441 mmol) were dissolved in acetonitrile (882 mL). To this was then added DBU (33.0 mL, 220 mmol). The resulting clear orange brown mixture was stirred at ambient temperature for 15 hours. The reaction mixture was concentrated down to remove solvents and afforded a dark reddish-orange oil. Solid crystals formed within a few hours at ambient temperature. This was isolated by washing with cold Et₂O and cold EtOAc (carefully to prevent dissolution) to afford 110 g (64% yield) of the title compound. The recrystallization was repeated to give another 13.7 g (8% yield). Additional compound was isolated by purification of the filtrate from the above recrystallization. This was purified by silica chromatography eluting with a 20-50% EtOAc/Hexanes gradient to afford an additional 22.7 g (13%) of the title compound. MS (apci) m/z=289.2 (M+H−Boc).

### Preparation E

### 7-Chloroimidazo[1,2-c]pyrimidin-5 (6H)-one

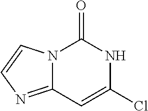 [SMILES: O=c1[nH]c(Cl)cc2nccn12]

### Step A: Preparation of 7-chloro-5-(methylthio)imidazo[1,2-c]pyrimidine hydrochloride

A solution of 6-chloro-2-(methylthio)pyrimidin-4-amine (25.17 g, 143.3 mmol) and 2-chloroacetaldehyde (27.73 mL, 215.0 mmol) (50% aqueous) in 1,4-dioxane (50 mL) was heated at 95° C. for 14 hours. The reaction mixture was allowed to cool to ambient temperature and then cooled in an ice bath. The reaction mixture was filtered and the solids washed with dioxane to afford 7-chloro-5-(methylthio)imidazo[1,2-c]pyrimidine hydrochloride (24.01 g, 101.7 mmol, 70.96% yield) as a tan powder. MS (apci) m/z=200.0 (M+H).

### Step B: Preparation of 7-Chloroimidazo[1,2-c]pyrimidin-5(6H)-one

7-Chloro-5-(methylthio)imidazo[1,2-c]pyrimidine hydrochloride (10.5 g, 44.5 mmol) was partially dissolved in MeOH (40 mL) and then a solution of potassium hydroxide (11.2 g, 200 mmol) in water (100 mL) was slowly added and the reaction was heated to reflux. The reaction generates methane thiol, so caution was taken to contain this noxious gas in the hood. After 2 hours the reaction was cooled and then neutralized with a solution of 1N HCl to reach a pH of between 6 and 7. The reaction was filtered and the solid was washed with MeOH. The solids were dried on the filter cake and then dried on a high vacuum pump to provide 7-chloroimidazo[1,2-c]pyrimidin-5(6H)-one (6.6 g, 87% yield) as a white solid. MS (apci) m/z=170.1 (M+H).

### Example 1

### ethyl 2-(3-(4-(7-(1-methyl-1H-pyrazol-4-yl)imidazo[1,2-c]pyrimidin-5-yl)-1H-pyrazol-1-yl)-1-(trifluoromethylsulfonyl)azetidin-3-yl)acetate

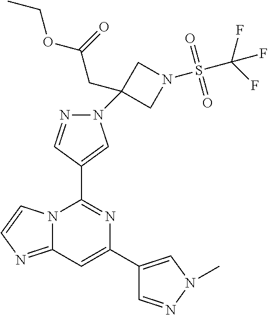 [SMILES: CCOC(=O)CC1(n2cc(-c3nc(-c4cnn(C)c4)cc4nccn34)cn2)CN(S(=O)(=O)C(F)(F)F)C1]

### Step A: 7-(1-methyl-1H-pyrazol-4-yl)-5-(1H-pyrazol-4-yl)imidazo[1,2-c]pyrimidine

(10.00 g, 37.7 mmol), tert-butyl 3-(2-ethoxy-2-oxoethylidene)azetidine-1-carboxylate (11.824 g, 49.006 mmol) and DBU (2.82 mL, 18.8 mmol) were suspended in CH₃CN (100 mL) in a glass bomb and heated at 60° C. overnight. The solids were collected by filtration and washed with MeCN and dried under high vacuum to furnish tert-butyl 3-(2-ethoxy-2-oxoethyl)-3-(4-(7-(1-methyl-1H-pyrazol-4-yl)imidazo[1,2-c]pyrimidin-5-yl)-1H-pyrazol-1-yl)azetidine-1-carboxylate (13.80 g, 27.2 mmol, 72.3% yield).

### Step B: tert-butyl 3-(2-ethoxy-2-oxoethyl)-3-(4-(7-(1-methyl-1H-pyrazol-4-yl)imidazo[1,2-c]pyrimidin-5-yl)-1H-pyrazol-1-yl)azetidine-1-carboxylate

(4.00 g, 7.90 mmol) was dissolved in 40 mL of DCM, followed by addition of HCl (19.7 mL, 79.0 mmol) 4.0 M in dioxane. The reaction was then stirred overnight at ambient temperature and then concentrated in vacuo. The residue was treated with saturated aqueous NaHCO₃ and extricated into ethyl acetate, dried and concentrated in vacuo, to afford ethyl 2-(3-(4-(7-(1-methyl-1H-pyrazol-4-yl)imidazo[1,2-c]pyrimidin-5-yl)-1H-pyrazol-1-yl)azetidin-3-yl)acetate (2.97 g, 7.31 mmol, 92.5% yield) as a light yellow oil.

### Step C: Ethyl 2-(3-(4-(7-(1-methyl-1H-pyrazol-4-yl)imidazo[1,2-c]pyrimidin-5-yl)-1H-pyrazol-1-yl)azetidin-3-yl)acetate

(1.5 g, 3.69 mmol) was suspended in DCM (100 mL) and DIEA (6.4 mL, 36.9 mmol) and DMAP (0.0451 g, 0.369 mmol) added and the reaction mixture stirred at 0° C. for 30 minutes. Triflic anhydride (0.931 ml, 5.53 mmol) was added dropwise and the stirred at 0° C. for 1 hour. The reaction mixture was quenched with saturated aqueous NaHCO₃ and diluted with DCM. The layers were separated and the combined organic layers dried, MgSO₄ and concentrated under reduced pressure to afford the crude material, which was purified by flash column chromatography (eluant: 1-4% 9:1 MeOH:NH₄OH/DCM) to furnish ethyl 2-(3-(4-(7-(1-methyl-1H-pyrazol-4-yl)imidazo[1,2-c]pyrimidin-5-yl)-1H-pyrazol-1-yl)-1-(trifluoromethylsulfonyl)azetidin-3-yl)acetate.

### Example 2

### 2-(3-(4-(7-(1-methyl-1H-pyrazol-4-yl)imidazo[1,2-c]pyrimidin-5-yl)-1H-pyrazol-1-yl)-1-((trifluoromethyl)sulfonyl)azetidin-3-yl)acetic acid

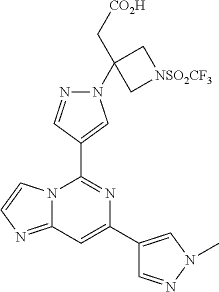

Ethyl 2-(3-(4-(7-(1-methyl-1H-pyrazol-4-yl)imidazo[1,2-c]pyrimidin-5-yl)-1H-pyrazol-1-yl)-1-(trifluoromethylsulfonyl)azetidin-3-yl)acetate (0.500 g, 0.93 mmol) was suspended in THF (20 mL) MeOH (5 mL) and LiOH (0.60 ml, 1.21 mmol) added and the reaction mixture stirred at ambient temperature for 2 hours. The reaction mixture acidified with 10% citratic acid and then partitioned between DCM and water. The aqueous was re-extracted with DCM (and a few drops of MeOH) and the combined organic layers dried, MgSO₄ and concentrated under reduced pressure to afford the crude 2-(3-(4-(7-(1-methyl-1H-pyrazol-4-yl)imidazo[1,2-c]pyrimidin-5-yl)-1H-pyrazol-1-yl)-1-(trifluoromethylsulfonyl)azetidin-3-yl)acetic acid (0.249 g, 0.49 mmol, 52.5% yield). MS (apci) m/z=511.1 (M+H).

### Example 3

### 2-(3-(4-(7-(1-methyl-1H-pyrazol-4-yl)imidazo[1,2-c]pyrimidin-5-yl)-1H-pyrazol-1-yl)-1-((trifluoromethyl)sulfonyl)azetidin-3-yl)ethanol

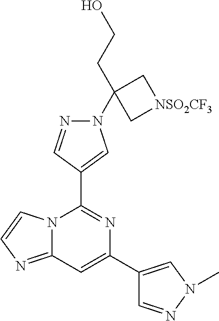

Ethyl 2-(3-(4-(7-(1-methyl-1H-pyrazol-4-yl)imidazo[1,2-c]pyrimidin-5-yl)-1H-pyrazol-1-yl)-1-(trifluoromethylsulfonyl)azetidin-3-yl)acetate (0.200 g, 0.371 mmol) was suspended in a 1:1 mixture of EtOH/THF (10 mL), and LiBH₄ (0.0162 g, 0.743 mmol) was added portionwise. The reaction mixture was stirred at ambient temperature until all of the borohydride was in solution and the gas evolution had stopped. The flask was then sealed and the reaction mixture was heated at 50° C. for 2 hours. LC-MS analysis showed some conversion. Additional LiBH₄ was added and the system was heated at 50° C. overnight. LC-MS analysis showed complete conversion to the desired mass. The reaction mixture was partitioned between saturated aqueous NH₄Cl and EtOAc. The combined organic layers were dried over MgSO₄ and concentrated under reduced pressure to afford the crude material, which was purified by flash column chromatography (eluant: 1-3% of a 9:1 mixture of MeOH:NH₄OH/DCM) to furnish 2-(3-(4-(7-(1-methyl-1H-pyrazol-4-yl)imidazo[1,2-c]pyrimidin-5-yl)-1H-pyrazol-1-yl)-1-(trifluoromethylsulfonyl)azetidin-3-yl)ethanol (0.105 g, 0.212 mmol, 57% yield). MS (apci) m/z=497.1 (M+H).

### Example 4

### 5-(1-(3-(2-methoxyethyl)-1-((trifluoromethyl)sulfonyl)azetidin-3-yl)-1H-pyrazol-4-yl)-7-(1-methyl-1H-pyrazol-4-yl)imidazo[1,2-c]pyrimidine

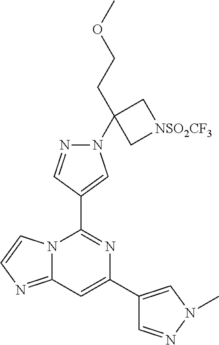

2-(3-(4-(7-(1-Methyl-1H-pyrazol-4-yl)imidazo[1,2-c]pyrimidin-5-yl)-1H-pyrazol-1-yl)-1-(trifluoromethylsulfonyl)azetidin-3-yl)ethanol (0.100 g, 0.201 mmol) was suspended in DMA (5 mL), and NaH (0.0121 g, 0.302 mmol) added portionwise. The reaction mixture was stirred at ambient temperature until complete deprotonation was observed. MeI (0.0251 mL, 0.408 mmol) was added and the reaction mixture stirred at ambient temperature overnight. The reaction mixture was partitioned between saturated aqueous NH₄Cl and EtOAc. The combined organic layers were washed with brine, dried, MgSO₄ and concentrated under reduced pressure to afford the crude material, which was purified by flash column chromatography (eluant 1-3% mixture of 9:1 MeOH:NH₄OH/DCM) to furnish 5-(1-(3-(2-methoxyethyl)-1-(trifluoromethylsulfonyl)azetidin-3-yl)-1H-pyrazol-4-yl)-7-(1-methyl-1H-pyrazol-4-yl)imidazo[1,2-c]pyrimidine (0.083 g, 0.163 mmol, 80.7% yield). MS (apci) m/z=511.1 (M+H).

### Example 5

### 5-(1-ethyl-1H-pyrazol-4-yl)-7-(1-methyl-1H-pyrazol-4-yl)imidazo[1,2-c]pyrimidine

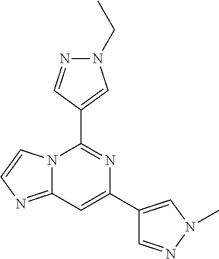 [SMILES: CCn1cc(-c2nc(-c3cnn(C)c3)cc3nccn23)cn1]

A suspension of 7-(1-methyl-1H-pyrazol-4-yl)-5-(1H-pyrazol-4-yl)imidazo[1,2-c]pyrimidine (0.40 g, 1.508 mmol) in anhydrous acetonitrile (10 ml) was treated at ambient temperature with 3-cyclopropylbut-2-enenitrile (0.4847 g, 4.524 mmol) followed by DBU (0.24 ml, 1.605 mmol). The crude material was purified by flash column chromatography. The title compound, which was a by-product of the reaction was isolated in 1% yield (0.0061 g). (MS (apci) m/z=294.3 (M+H).

### Example 6

### 2-cyclopropyl-2-(4-(7-(1-methyl-1H-pyrazol-4-yl)imidazo[1,2-c]pyrimidin-5-yl)-1H-pyrazol-1-yl)ethanol

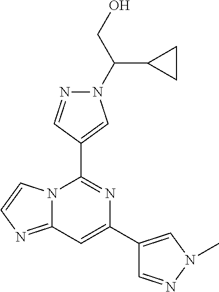 [SMILES: Cn1cc(-c2cc3nccn3c(-c3cnn(C(CO)C4CC4)c3)n2)cn1]

Step A: To ethyl 2-(tert-butyldimethylsilyloxy)acetate (7.75 g, 35.5 mmol) and N,O-dimethylhydroxylamine hydrochloride (7.27 g, 74.5 mmol) in 400 mL THF cooled in ice, was added dropwise a 2.0M solution of isopropylmagnesium chloride in THF (71.0 mL, 142 mmol). The mixture was allowed to slowly warm to ambient temperature. The reaction mixture was quenched with aqueous NH₄Cl and concentrated to ⅓ volume. The residue was diluted with water and extracted with EtOAc. The EtOAc was washed with brine, dried over MgSO₄, filtered, and evaporated to yield 2-(tert-butyldimethylsilyloxy)-N-methoxy-N-methylacetamide (7.50 g, 32.1 mmol, 90.5% yield) as a pale yellow oil. ¹HNMR (CDCl₃) and LC/MS were consistent with the desired structure.

Step B: To a solution of 2-(tert-butyldimethylsilyloxy)-N-methoxy-N-methylacetamide (1.50 g, 6.43 mmol) in 15 mL THF cooled in ice was added dropwise 0.5M cyclopropylmagnesium bromide in THF (22.5 mL, 11.2 mmol). The clear yellow solution became turbid after 5 minutes. The suspension was stirred in an ice-bath for 80 minutes. The reaction mixture was then quenched with saturated aqueous NH₄Cl and concentrated. The aqueous residue was partitioned between water and DCM. The aqueous layer was extracted with another portion of DCM. The DCM layers were dried over MgSO₄, filtered, and evaporated to yield 1.56 g yellow oil. The oil was purified on a 50 g Biotage SNAP column with 20:1 hexane/EtOAc, affording 2-(tert-butyldimethylsilyloxy)-1-cyclopropylethanone (0.61 g, 2.85 mmol, 44.3% yield) as a colorless oil.

Step C: To 2-(tert-butyldimethylsilyloxy)-1-cyclopropylethanone (0.61 g, 2.8 mmol) in 6 mL methanol cooled in an ice-bath, was added sodium borohydride (0.065 g, 1.7 mmol), and the reaction mixture was stirred for 2.5 hours. The reaction mixture was treated with 4 mL saturated aqueous NH₄Cl, 4 mL 1M HCl, 50 mL DCM, stirred for 5 minutes, diluted with water, and the layers was separated. The aqueous layer was extracted with another portion of DCM. The combined DCM layers were dried over MgSO₄, filtered, and evaporated to yield 0.52 g of crude material as a colorless oil. The crude material was chromatographed on a 50 g Biotage SNAP column with 10:1 hexane:EtOAc, affording 2-(tert-butyldimethylsilyloxy)-1-cyclopropylethanol (0.44 g, 2.0 mmol, 71% yield) as colorless oil.

Step D: To 2-(tert-butyldimethylsilyloxy)-1-cyclopropylethanol (0.050 g, 0.23 mmol) in 5 mL DCM cooled in ice was added triethylamine (0.048 mLml, 0.35 mmol) and 1,4-diazabicyclo[2.2.2]octane (0.008 g, 0.069 mmol). To this was added methanesulfonyl chloride (0.022 mL, 0.28 mmol). The clear solution was stirred at ice bath temperature. After 45 minutes, the reaction mixture was washed with aqueous NaHCO₃, dried over MgSO₄, filtered, and evaporated to yield 2-(tert-butyldimethylsilyloxy)-1-cyclopropylethyl methanesulfonate (0.063 g, 0.214 mmol, 93% yield) as a colorless oil.

Step E: To a vial containing 7-(1-methyl-1H-pyrazol-4-yl)-5-(1H-pyrazol-4-yl)imidazo[1,2-c]pyrimidine (0.037 g, 0.139 mmol) in 1.0 mL DMF cooled in ice was added 60% sodium hydride (0.006 g, 0.160 mmol). The mixture was stirred at ambient temperature for 10 minutes, and then a solution of 2-(tert-butyldimethylsilyloxy)-1-cyclopropylethyl methanesulfonate (0.0616 g, 0.209 mmol) in 1 mL DMF was added. The vial was sealed and heated at 60° C. The reaction mixture was partitioned between water and EtOAc. The EtOAc was washed with water, brine, dried over MgSO4, filtered, and evaporated to yield 46.2 mg of crude material. The crude material was chromatographed on a 10 g Biotage SNAP column with 10:1 EtOAc:MeOH, affording 2.9 mg of 5-(1-(2-(tert-butyldimethylsilyloxy)-1-cyclopropylethyl)-1H-pyrazol-4-yl)-7-(1-methyl-1H-pyrazol-4-yl)imidazo[1,2-c]pyrimidine as a colorless film

Step F: To 5-(1-(2-(tert-butyldimethylsilyloxy)-1-cyclopropylethyl)-1H-pyrazol-4-yl)-7-(1-methyl-1H-pyrazol-4-yl)imidazo[1,2-c]pyrimidine (0.0029 g, 0.0063 mmol) in 2 mL THF was added 1M TBAF in THF (0.019 mL, 0.019 mmol), and the mixture was stirred at ambient temperature for 1 hour. The reaction mixture was concentrated and the residue was partitioned between water and EtOAc. The EtOAc was washed with brine, dried over MgSO₄, filtered, and concentrated. The crude material was chromatographed on a 10 g Biotage SNAP column with 8:1 EtOAc:MeOH, affording 2-cyclopropyl-2-(4-(7-(1-methyl-1H-pyrazol-4-yl)imidazo[1,2-c]pyrimidin-5-yl)-1H-pyrazol-1-yl)ethanol (0.0008 g, 0.0023 mmol, 37% yield) MS (apci) m/z=350.2 (M+H).

### Example 7

### 7-(1-Methyl-1H-pyrazol-4-yl)-5-(1-(pent-3-yn-1-yl)-1H-pyrazol-4-yl)imidazo[1,2-c]pyrimidine

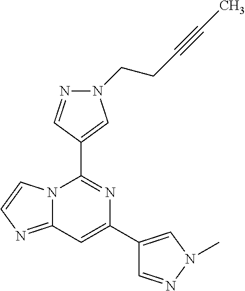 [SMILES: CC#CCCn1cc(-c2nc(-c3cnn(C)c3)cc3nccn23)cn1]

7-(1-Methyl-1H-pyrazol-4-yl)-5-(1H-pyrazol-4-yl)imidazo[1,2-c]pyrimidine (0.198 g, 0.746 mmol) was added to a mixture of triphenylphosphine (0.587 g, 2.24 mmol) and pent-3-yn-1-ol (0.207 g, 2.46 mmol) in dissolved in THF (1.9 mL). The mixture was heated to 60° C. and the hot solution was treated dropwise with a solution of diisopropyl azodicarboxylate (0.441 ml, 2.240 mmol) dissolved in toluene (1.2 mL). Near the end of the addition, the mixture became homogeneous. After the addition, the mixture was heated at 60° C. for an additional 2 hours. The reaction was cooled and concentrated in vacuo then applied directly onto a silica gel column using methylene chloride. The column was eluted with a gradient of (2% NH4OH in isopropanol)/methylene chloride. The desired product was recovered as an off-white solid, (225 mg, 91%). APCI MS (+) m/z 332.2 (M+H)+.

### Example 8

### 5-(1-(But-3-yn-1-yl)-1H-pyrazol-4-yl)-7-(1-methyl-1H-pyrazol-4-yl)imidazo[1,2-c]pyrimidine

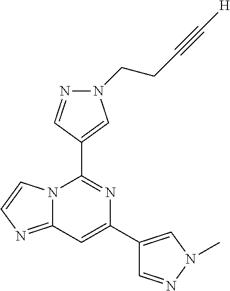 [SMILES: C#CCCn1cc(-c2nc(-c3cnn(C)c3)cc3nccn23)cn1]

7-(1-Methyl-1H-pyrazol-4-yl)-5-(1H-pyrazol-4-yl)imidazo[1,2-c]pyrimidine (0.198 g, 0.7464 mmol) was added to a solution of triphenylphosphine (0.587 g, 2.24 mmol) and but-3-yn-1-ol (0.173 g, 2.46 mmol) dissolved in THF (1.9 mL). The mixture was heated to 60° C. and the hot solution was treated dropwise with a solution of diisopropyl azodicarboxylate (0.441 ml, 2.24 mmol) dissolved in toluene (1.2 mL). Near the end of the addition, the mixture became homogeneous. After the addition, the mixture was heated at 60° C. for an additional 3 hours. The reaction was cooled and applied directly onto a silica gel column using methylene chloride. The column was eluted with a gradient of (2% NH₄OH in isopropanol)/methylene chloride. The desired product was isolated as an off-white solid (231 mg, 97%). APCI MS (+) m/z 318.3. (M+H)⁺.

### Example 9

### 5-(1-(Dicyclopropylmethyl)-1H-pyrazol-4-yl)-7-(1-methyl-1H-pyrazol-4-yl)imidazo[1,2-c]pyrimidine

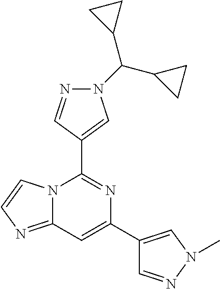 [SMILES: Cn1cc(-c2cc3nccn3c(-c3cnn(C(C4CC4)C4CC4)c3)n2)cn1]

7-(1-Methyl-1H-pyrazol-4-yl)-5-(1H-pyrazol-4-yl)imidazo[1,2-c]pyrimidine (0.198 g, 0.746 mmol) was added to a solution of triphenylphosphine (0.587 g, 2.24 mmol) and dicyclopropylmethanol (0.276 g, 2.46 mmol) dissolved in THF (1.9 mL). The mixture was heated to 60° C. and the hot solution was treated dropwise with a solution of diisopropyl azodicarboxylate (0.441 mL, 2.24 mmol) dissolved in toluene (1.2 mL). Near the end of the addition, the mixture became homogeneous. After the addition, the mixture was heated at 60° C. for 20 hours. The mixture was cooled and concentrated to a thick syrup. This syrup was dissolved in methylene chloride and washed with water, dried over Na₂SO₄ and concentrated in vacuo. The residue was applied to a silica gel column eluted with a gradient of (2% NH₄OH in IPA)/methylene chloride. The product was isolated as a yellow oil, which slowly solidified (206 mg, 77%). APCI MS (+) m/z 360.2 (M+H)⁺.

### Example 10

### 5-(1-(1-Cyclopropylpent-4-en-2-yl)-1H-pyrazol-4-yl)-7-(1-methyl-1H-pyrazol-4-yl)imidazo[1,2-c]pyrimidine

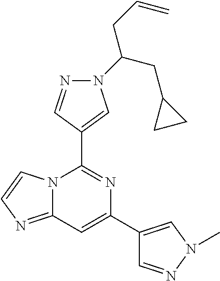 [SMILES: C=CCC(CC1CC1)n1cc(-c2nc(-c3cnn(C)c3)cc3nccn23)cn1]

Step A: Zinc-Copper couple (17.8 g, 111 mmol) was slurried in diethyl ether (42 mL) and treated with iodine crystals (0.182 g, 0.719 mmol). The mixture was stirred until the brown color faded. A mixture of hepta-1,6-dien-4-ol (6.2 g, 55.3 mmol) and diiodomethane (8.92 ml, 111 mmol) was added dropwise, and the suspension was stirred while heating at a gentle reflux for 60 hours. The mixture was cooled, diluted with additional diethyl ether, treated with celite, then filtered the mixture through a bed of celite. The filtrate was washed with cold 5% HCl, cold water, saturated NaHCO₃, saturated NaCl, dried over Na₂SO₄, filtered and concentrated to an amber oil. The crude oil was evaporatively distilled and the fraction boiling between 80-90° C. at 0.6 mm Hg was collected. The colorless oil (393 mg) contained a mixture of 1,3-dicyclopropylpropan-2-ol and 1-cyclopropylpent-4-en-2-ol as confirmed by proton NMR.

Step B: 7-(1-Methyl-1H-pyrazol-4-yl)-5-(1H-pyrazol-4-yl)imidazo[1,2-c]pyrimidine (0.248 g, 0.935 mmol) was combined with triphenylphosphine (0.736 g, 2.80 mmol) and a mixture of 1,3-dicyclopropylpropan-2-ol and 1-cyclopropylpent-4-en-2-ol (0.393 g, 2.80 mmol) in THF (2.4 mL). The mixture was heated to 60° C. and the hot solution was treated dropwise with a solution of diisopropyl azodicarboxylate (0.552 mL, 2.80 mmol) dissolved in toluene (1.5 mL). Near the end of the addition, the mixture became homogeneous. After the addition, the mixture was heated at 60° C. for 2 hours. After cooling, the mixture was diluted with methylene chloride and washed with water, dried over Na₂SO₄ and concentrated in vacuo. The residue was chromatographed on SiO₂, eluting with a gradient of (2% NH₄OH in isopropanol)/methylene chloride. The isolated material was a mixture of two products. This mixture was purified on reversed-phase silica gel (Phenomenex Luna 5 u C18(2), 100 Å, Axia Pac, 150×21.2 mm, 5 micron column, 5-95% gradient of (water+0.1% TFA) and (MeCN+0.1% TFA) using a single injection. 5-(1-(1-Cyclopropylpent-4-en-2-yl)-1H-pyrazol-4-yl)-7-(1-methyl-1H-pyrazol-4-yl)imidazo[1,2-c]pyrimidine was isolated as the first eluting peak (28.3 mg, 8%). APCI MS (+) m/z 374.2 (M+H)⁺.

### Example 11

### 5-(1-(1,3-Dicyclopropylpropan-2-yl)-1H-pyrazol-4-yl)-7-(1-methyl-1H-pyrazol-4-yl)imidazo[1,2-c]pyrimidine

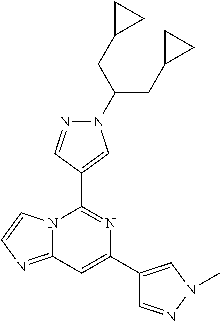 [SMILES: Cn1cc(-c2cc3nccn3c(-c3cnn(C(CC4CC4)CC4CC4)c3)n2)cn1]

Step A: Zinc-Copper couple (17.8 g, 111 mmol) was slurried in diethyl ether (42 mL) and treated with iodine crystals (0.182 g, 0.719 mmol) and the mixture was stirred until the brown color faded. A mixture of hepta-1,6-dien-4-ol (6.2 g, 55.3 mmol) and diiodomethane (8.92 ml, 111 mmol) was added to the mixture dropwise, and the suspension was stirred while heating at a gentle reflux for 60 hours. The mixture was cooled, diluted with additional diethyl ether, treated with Celite®, and then filtered through a bed of Celite®. The filtrate was washed with cold 5% HCl, cold water, saturated NaHCO₃, saturated NaCl, dried over Na₂SO₄ and concentrated to an amber oil. The crude oil was evaporatively distilled and the fraction boiling between 80-90° C. at 0.6 mm Hg was collected. The colorless oil (393 mg) was a mixture of 1,3-dicyclopropylpropan-2-ol and 1-cyclopropylpent-4-en-2-ol as confirmed by proton NMR.

Step B: 7-(1-Methyl-1H-pyrazol-4-yl)-5-(1H-pyrazol-4-yl)imidazo[1,2-c]pyrimidine (0.248 g, 0.935 mmol) was combined with triphenylphosphine (0.736 g, 2.80 mmol) and a mixture of 1,3-dicyclopropylpropan-2-ol and 1-cyclopropylpent-4-en-2-ol (0.393 g, 2.80 mmol) in THF (2.4 mL). The mixture was heated to 60° C. and the hot solution was treated dropwise with a solution of diisopropyl azodicarboxylate (0.552 mL, 2.80 mmol) dissolved in toluene (1.5 mL). Near the end of the addition, the mixture became homogeneous. After the addition, the mixture was heated at 60° C. for 2 hours. After cooling, the mixture was diluted with methylene chloride and washed with water, dried over Na₂SO₄ and concentrated in vacuo. The residue was chromatographed on SiO₂, eluting with a gradient of (2% NH₄OH in isopropanol)/methylene chloride. The isolated material was a mixture of two products. This mixture was purified on reversed-phase silica gel (Phenomenex Luna 5 u C18(2), 100 Å, Axia Pac, 150×21.2 mm, 5 micron column, 5-95% gradient of (water+0.1% TFA) and (MeCN+0.1% TFA) using a single injection. The desired product was isolated as the second eluting peak (44.3 mg, 12%). APCI MS (+) m/z 388.3 (M+H)⁺.

### Example 12

### Ethyl 3-cyclopropyl-3-(4-(7-(1-methyl-1H-pyrazol-4-yl)imidazo[1,2-c]pyrimidin-5-yl)-1H-pyrazol-1-yl)propanoate

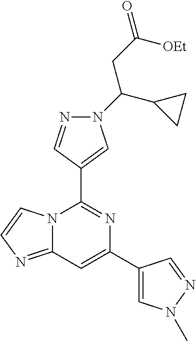 [SMILES: CCOC(=O)CC(C1CC1)n1cc(-c2nc(-c3cnn(C)c3)cc3nccn23)cn1]

### Step A: Preparation of Ethyl 3-cyclopropylacrylate

Ethyl 2-(diethoxyphosphoryl)acetate (39.83 mL, 200.7 mmol) was dissolved in THF (300 ml) and cooled to 0° C. Sodium hydride (8.029 g, 200.7 mmol) was added portion-wise and the reaction warmed to ambient temperature. After 1 hour cyclopropane carboxaldehyde (10.00 mL, 133.8 mmol) was added drop-wise and the reaction allowed to stir at ambient temperature for 2 hours. The reaction mixture was diluted with saturated aqueous NaHCO₃ and EtOAc. The combined organic layers were washed with brine, dried over MgSO₄ and concentrated under reduced pressure to afford the crude material. The crude material was purified by a silica gel plug eluting with 75% hexanes/CH₂Cl₂ to afford 18.5 g (97%) of the desired product as a colorless oil. The product was determined to be a 97:3 mixture of the E:Z isomers by ¹H NMR.

### Step B: Preparation of Ethyl 3-cyclopropyl-3-(4-(7-(1-methyl-1H-pyrazol-4-yl)imidazo[1,2-c]pyrimidin-5-yl)-1H-pyrazol-1-yl)propanoate

To a sealable flask was added 7-(1-methyl-1H-pyrazol-4-yl)-5-(1H-pyrazol-4-yl)imidazo[1,2-c]pyrimidine (5.0 g, 18.8 mmol), (E)-ethyl 3-cyclopropylacrylate (5.28 g, 37.7 mmol), acetonitrile (62.8 mL) and DBU (1.42 mL, 9.42 mmol). The flask was sealed and the mixture was stirred at 100° C. for 16 hours. The mixture was cooled to ambient temperature and concentrated under reduced pressure to afford the crude material, which was purified by flash column chromatography (2-6% MeOH/DCM) to provide 5.26 g (67%) of the desired product as an off-white powder. MS APCI (+) m/z 406.2 (M+1) detected.

### Example 13

### 3-cyclopropyl-3-(4-(7-(1-methyl-1H-pyrazol-4-yl)imidazo[1,2-c]pyrimidin-5-yl)-1H-pyrazol-1-yl)propan-1-ol

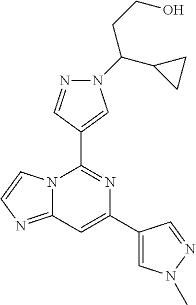 [SMILES: Cn1cc(-c2cc3nccn3c(-c3cnn(C(CCO)C4CC4)c3)n2)cn1]

To a solution of ethyl 3-cyclopropyl-3-(4-(7-(1-methyl-1H-pyrazol-4-yl)imidazo[1,2-c]pyrimidin-5-yl)-1H-pyrazol-1-yl)propanoate (0.250 g, 0.617 mmol) in THF (2.06 mL) and ethanol (2.06 mL) was added LiBH₄ (0.0269 g, 1.23 mmol). The mixture was then warmed to ambient temperature where it stirred for 1 hour. The mixture was then warmed to 50° C. where it stirred for 3 hours. The mixture was cooled to ambient temperature and concentrated. The mixture was treated with saturated aqueous NH₄Cl and extracted with CH₂Cl₂. The combined organic extracts were dried over Na₂SO₄, filtered and concentrated. The crude product was purified by silica gel column chromatography (3 to 10% MeOH/CH₂Cl₂ with 6% NH₄OH/MeOH) to afford 0.101 g (44%) of the product as a white solid. MS APCI (+) m/z 364.2 (M+1) detected.

### Example 14

### 5-(1-(1-Cyclopropylethyl)-1H-pyrazol-4-yl)-7-(1-methyl-1H-pyrazol-4-yl)imidazo[1,2-c]pyrimidine

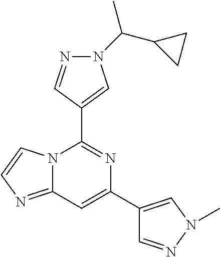 [SMILES: CC(C1CC1)n1cc(-c2nc(-c3cnn(C)c3)cc3nccn23)cn1]

7-(1-Methyl-1H-pyrazol-4-yl)-5-(1H-pyrazol-4-yl)imidazo[1,2-c]pyrimidine (213.2 mg, 0.804 mmol) was dissolved in THF (0.2 M) and treated with alpha-methylcyclopropanemethanol (207.7 mg, 2.411 mmol) and triphenylphosphine (632.4 mg, 2.411 mmol) and the reaction mixture was heated to 60° C. The reaction mixture was then treated with diethyl azodicarboxylate (1.10 mL, 2.411 mmol, 40% wt) and stirred at 60° C. for 4 hours then cooled to ambient temperature and concentrated in vacuo. Silica gel chromatography (DCM/IPA) of the crude material, followed by C18 chromatography (water/ACN) provided 5-(1-(1-cyclopropylethyl)-1H-pyrazol-4-yl)-7-(1-methyl-1H-pyrazol-4-yl)imidazo[1,2-c]pyrimidine (89.3 mg, 0.268 mmol, 33.3% yield). m/z (APCI-pos) M+1=334.1.

### Example 15

### 5-(1-(1-Cyclopropylbutyl)-1H-pyrazol-4-yl)-7-(1-methyl-1H-pyrazol-4-yl)imidazo[1,2-c]pyrimidine

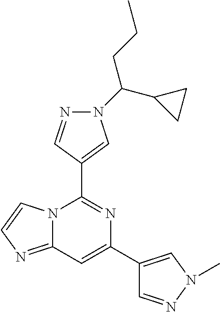 [SMILES: CCCC(C1CC1)n1cc(-c2nc(-c3cnn(C)c3)cc3nccn23)cn1]

Step A: In an oven dried flask, cyclopropanecarbaldehyde (2.0 g, 28.5 mmol) was dissolved in diethyl ether (0.4 M) and placed under an atmosphere of N₂. The reaction mixture was cooled to 0° C., and treated dropwise with propylmagnesium chloride (21.4 mL, 42.8 mmol, 2.0 M). The reaction mixture was stirred at 0° C. for 1 hour, then quenched by pouring the reaction mixture onto ice. The mixture was extracted with diethyl ether, washed with water and brine, dried over Na₂SO₄ and filtered. The crude material was purified by distillation to provide 1-cyclopropylbutan-1-ol (2.80 g, 24.5 mmol, 85.9% yield).

Step B: 7-(1-Methyl-1H-pyrazol-4-yl)-5-(1H-pyrazol-4-yl)imidazo[1,2-c]pyrimidine (203.1 mg, 0.766 mmol) was dissolved in THF (0.2 M) and treated with 1-cyclopropylbutan-1-ol (262.3 mg, 2.297 mmol) and triphenylphosphine (602.4 mg, 2.297 mmol) and heated to 60° C. The reaction mixture was then treated with diethyl azodicarboxylate (1.10 mL, 2.297 mmol, 40% wt) and stirred for 4 hours. The reaction mixture was cooled to ambient temperature and concentrated in vacuo. Silica gel chromatography (DCM/IPA) of the crude material followed by C18 chromatography (water/ACN) provided 5-(1-(1-cyclopropylbutyl)-1H-pyrazol-4-yl)-7-(1-methyl-1H-pyrazol-4-yl)imidazo[1,2-c]pyrimidine (100.6 mg, 0.278 mmol, 36.4% yield). m/z (APCI-pos) M+1=362.2.

### Example 16

### 5-(1-(3-Methyl-1-(trifluoromethylsulfonyl)azetidin-3-yl)-1H-pyrazol-4-yl)-7-(1-methyl-1H-pyrazol-4-yl)imidazo[1,2-c]pyrimidine

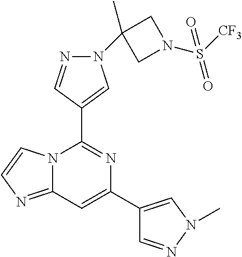 [SMILES: Cn1cc(-c2cc3nccn3c(-c3cnn(C4(C)CN(S(=O)(=O)C(F)(F)F)C4)c3)n2)cn1]

Step A: In an oven dried flask, tert-butyl 3-oxoazetidine-1-carboxylate (1.01 g, 5.900 mmol) was dissolved in diethyl ether (0.2 M) and place under an atmosphere of N₂. The reaction mixture was cooled to 0° C. and treated dropwise with methylmagnesium bromide (2.07 mL, 6.195 mmol, 3.0 M). The reaction mixture was stirred at 0° C. for 1 hour then quenched by pouring onto ice. The mixture was extracted with diethyl ether, washed with water and brine, dried over Na₂SO₄, filtered, and concentrated in vacuo to provide tert-butyl 3-hydroxy-3-methylazetidine-1-carboxylate (865.3 mg, 4.621 mmol, 78.3% yield).

Step B: 7-(1-Methyl-1H-pyrazol-4-yl)-5-(1H-pyrazol-4-yl)imidazo[1,2-c]pyrimidine (269.0 mg, 1.01 mmol) was dissolved in THF (0.1 M) and treated with tert-butyl 3-hydroxy-3-methylazetidine-1-carboxylate (570.0 mg, 3.04 mmol) and triphenylphosphine (798.0 mg, 3.04 mmol) and heated to 60° C. The reaction mixture was then treated with diethyl azodicarboxylate (1.40 mL, 3.04 mmol, 40% wt) and stirred for 24 hours. The reaction mixture was cooled to ambient temperature and concentrated in vacuo. Silica gel chromatography (DCM/IPA) followed by C18 chromatography (water/ACN) provided tert-butyl 3-methyl-3-(4-(7-(1-methyl-1H-pyrazol-4-yl)imidazo[1,2-c]pyrimidin-5-yl)-1H-pyrazol-1-yl)azetidine-1-carboxylate (82.3 mg, 0.095 mmol, 9.34% yield).

Step C: Tert-butyl 3-methyl-3-(4-(7-(1-methyl-1H-pyrazol-4-yl)imidazo[1,2-c]pyrimidin-5-yl)-1H-pyrazol-1-yl)azetidine-1-carboxylate (82.3 mg, 0.189 mmol) was dissolved in 4 N HCl in 1,4-dioxane (1.0 mL) and stirred at ambient temperature for 1 hour. The reaction mixture was concentrated in vacuo. The residue was dissolved in 4:1 DCM:IPA, washed with saturated NaHCO₃ and brine, dried over Na₂SO₄, filtered, and concentrated in vacuo to provide 7-(1-methyl-1H-pyrazol-4-yl)-5-(1-(3-methylazetidin-3-yl)-1H-pyrazol-4-yl)imidazo[1,2-c]pyrimidine (40.0 mg, 0.120 mmol, 63.2% yield).

Step D: 7-(1-M ethyl-1H-pyrazol-4-yl)-5-(1-(3-methylazetidin-3-yl)-1H-pyrazol-4-yl)imidazo[1,2-c]pyrimidine (40.0 mg, 0.120 mmol) was dissolved in DCM (0.1 M), cooled to 0° C., and then sequentially treated with N—N-diisopropylethylamine (104.2 μL, 0.598 mmol) and trifluoromethanesulfonic anhydride (30.2 μL, 0.179 mmol). The reaction mixture was warmed to ambient temperature and stirred for 24 hours. The reaction mixture was diluted with DCM, washed with saturated NaHCO₃, dried over Na₂SO₄, filtered, and concentrated in vacuo. Silica gel chromatography (DCM/IPA) of the crude material followed by C18 chromatography (water/ACN) provided 5-(1-(3-methyl-1-(trifluoromethylsulfonyl)azetidin-3-yl)-1H-pyrazol-4-yl)-7-(1-methyl-1H-pyrazol-4-yl)imidazo[1,2-c]pyrimidine (3.4 mg, 0.007 mmol, 5.5% yield). m/z (APCI-pos) M+1=467.1.

### Example 17

### 5-(1-(1-Cyclopropyl-3-fluoropropyl)-1H-pyrazol-4-yl)-7-(1-methyl-1H-pyrazol-4-yl)imidazo[1,2-c]pyrimidine

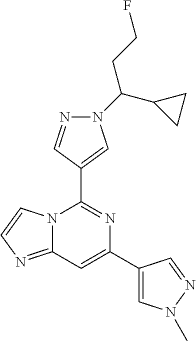 [SMILES: Cn1cc(-c2cc3nccn3c(-c3cnn(C(CCF)C4CC4)c3)n2)cn1]

Step A: Ethyl 3-cyclopropyl-3-(4-(7-(1-methyl-1H-pyrazol-4-yl)imidazo[1,2-c]pyrimidin-5-yl)-1H-pyrazol-1-yl)propanoate (423.4 mg, 1.044 mmol) was dissolved in 2:1 EtOH/THF (0.1 M), cooled to 0° C., and then treated with lithium borohydride (45.5 mg, 2.089 mmol). The reaction mixture was stirred at ambient temperature for 1 hour and then heated to 50° C. for 16 hours. The reaction mixture was cooled to ambient temperature and concentrated. The residue was quenched with saturated NH₄Cl, extracted with DCM, dried over Na₂SO₄, filtered and concentrated in vacuo. Silica gel chromatography (DCM/IPA) of the crude material provided 3-cyclopropyl-3-(4-(7-(1-methyl-1H-pyrazol-4-yl)imidazo[1,2-c]pyrimidin-5-yl)-1H-pyrazol-1-yl)propan-1-ol (260.9 mg, 0.718 mmol, 68.8% yield).

Step B: 3-Cyclopropyl-3-(4-(7-(1-methyl-1H-pyrazol-4-yl)imidazo[1,2-c]pyrimidin-5-yl)-1H-pyrazol-1-yl)propan-1-ol (168.3 mg, 0.463 mmol) was dissolved in DCM (0.1 M), cooled to 0° C., and treated sequentially with triethylamine (94.2 μL, 0.695 mmol) and methanesulfonic anhydride (96.8 mg, 0.556 mmol). The reaction mixture was stirred at ambient temperature for 1 hour and then quenched with saturated NaHCO₃. The combined organic layers were separated, dried over Na₂SO₄, filtered, and concentrated in vacuo to provide 3-cyclopropyl-3-(4-(7-(1-methyl-1H-pyrazol-4-yl)imidazo[1,2-c]pyrimidin-5-yl)-1H-pyrazol-1-yl)propyl methanesulfonate (178.2 mg, 0.404 mmol, 87.2% yield).

Step C: 3-Cyclopropyl-3-(4-(7-(1-methyl-1H-pyrazol-4-yl)imidazo[1,2-c]pyrimidin-5-yl)-1H-pyrazol-1-yl)propyl methanesulfonate (178.2 mg, 0.404 mmol) was dissolved in THF (0.1 M) and treated with 1.0 M tetrabutylammonium fluoride (807 μL, 0.807 mmol) and stirred at 60° C. for 1 hour. The reaction mixture was cooled to ambient temperature and concentrated in vacuo, and the crude material was purified by silica gel chromatography (DCM/IPA) to provide 5-(1-(1-cyclopropyl-3-fluoropropyl)-1H-pyrazol-4-yl)-7-(1-methyl-1H-pyrazol-4-yl)imidazo[1,2-c]pyrimidine (91.4 mg, 0.250 mmol, 62.0% yield). m/z (APCI-pos) M+1=366.1.

### Example 18

### 5-(1-(3-(2-Fluoroethyl)-1-(trifluoromethylsulfonyl)azetidin-3-yl)-1H-pyrazol-4-yl)-7-(1-methyl-1H-pyrazol-4-yl)imidazo[1,2-c]pyrimidine

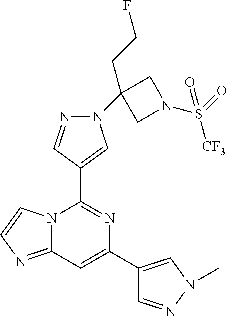 [SMILES: Cn1cc(-c2cc3nccn3c(-c3cnn(C4(CCF)CN(S(=O)(=O)C(F)(F)F)C4)c3)n2)cn1]

Step A: Tert-butyl 3-oxoazetidine-1-carboxylate (19.0 g, 111.0 mmol) was dissolved in THF (400 mL), cooled to 0° C., and treated portion wise with sodium hydride (6.66 g, 166.0 mmol, 60% wt). The reaction mixture was warmed to ambient temperature and treated drop wise with a 150 mL THF solution of triethyl phosphonoacetate (33.0 mL, 166.0 mmol) and stirred for 2 hours at ambient temperature. The reaction mixture was quenched with saturated NaHCO₃ and concentrated in vacuo. The residue was extracted with EtOAc, washed with saturated NaHCO₃ and brine, and the combined organic extracts were dried over Na₂SO₄, filtered and concentrated in vacuo. Silica gel chromatography (DCM/IPA with 2% NH₄OH) of the crude material provided tert-butyl 3-(2-ethoxy-2-oxoethylidene)azetidine-1-carboxylate (21.0 g, 87.0 mmol, 78.4% yield).

Step B: 4-Bromopyrazole (6.7 g, 45.59 mmol) was dissolved in ACN (0.3 M) and treated sequentially with tert-butyl 3-(2-ethoxy-2-oxoethylidene)azetidine-1-carboxylate (12.1 g, 51.15 mmol) and 1,8-diazabicyclo[5.4.0]undec-7-ene (7.50 mL, 50.15 mmol) and heated to 60° C. for 16 hours. The reaction mixture was cooled to ambient temperature and concentrated in vacuo. The crude product was purified by silica gel chromatography (Hexand/EtOAc) to provide tert-butyl 3-(4-bromo-1H-pyrazol-1-yl)-3-(2-ethoxy-2-oxoethyl)azetidine-1-carboxylate (14.5 g, 37.35 mmol, 81.9% yield).

Step C: Tert-butyl 3-(4-bromo-1H-pyrazol-1-yl)-3-(2-ethoxy-2-oxoethyl)azetidine-1-carboxylate (14.5 g, 37.3 mmol) was dissolved in THF (0.2 M), cooled to 0° C., and treated dropwise with diisobutylaluminum hydride (62.2 mL, 93.4 mmol, 1.5 M). The mixture was stirred at ambient temperature for 1 hour and then cooled back down to 0° C. and quenched by slow addition of 0.5 N sodium potassium tartrate. The mixture was filtered through GF/F paper, and the filtrate was concentrated in vacuo. The residue was diluted with EtOAc, washed with water and brine, dried over Na₂SO₄, filtered and concentrated in vacuo. Silica gel chromatography (Hexane/EtOAc) of the crude material provided tert-butyl 3-(4-bromo-1H-pyrazol-1-yl)-3-(2-hydroxyethyl)azetidine-1-carboxylate (7.3 g, 21.1 mmol, 56.5% yield).

Step D: Tert-butyl 3-(4-bromo-1H-pyrazol-1-yl)-3-(2-hydroxyethyl)azetidine-1-carboxylate (7.3 g, 21.1 mmol) was dissolved in DCM (0.2 M), cooled to 0° C., and then treated sequentially with triethylamine (8.63 mL, 63.3 mmol) and methanesulfonic anhydride (7.35 g, 42.2 mmol) and stirred at ambient temperature for 1 hour. The reaction mixture was diluted with additional DCM and washed with NaHCO₃, dried over Na₂SO₄, filtered and concentrated to provide tert-butyl 3-(4-bromo-1H-pyrazol-1-yl)-3-(2-(methylsulfonyloxy)ethyl)azetidine-1-carboxylate (8.95 g, 21.1 mmol, 100% yield).

Step E: Tert-butyl 3-(4-bromo-1H-pyrazol-1-yl)-3-(2-(methylsulfonyloxy)ethyl)azetidine-1-carboxylate (8.95 g, 21.1 mmol) was dissolved in THF (0.2 M) and treated with tetrabutylammonium fluoride (28.1 mL, 42.2 mmol, 1.5 M) and stirred at 60° C. for 2 hours. The reaction mixture was cooled to ambient temperature and concentrated. The residue was diluted with EtOAc, washed with water and brine, dried over Na₂SO₄, filtered and concentrated in vacuo. Purification of the crude material by silica gel chromatography provided tert-butyl 3-(4-bromo-1H-pyrazol-1-yl)-3-(2-fluoroethyl)azetidine-1-carboxylate (3.12 g, 8.96 mmol, 42.5% yield). m/z (APCI-pos) M+1−Boc=247.9

Step F: Tert-butyl 3-(4-bromo-1H-pyrazol-1-yl)-3-(2-fluoroethyl)azetidine-1-carboxylate (958.1 mg, 2.751 mmol) was dissolved in 1,4-dioxane (0.2 M) and treated with bis(pinacolato)diboron (768.6 mg, 3.027 mmol) and potassium acetate (810.1 mg, 8.254 mmol). The reaction mixture was degassed with argon and to this was added 1,1′-bis(diphenylphosphino)ferrocene palladium (II) chloride complex with dichloromethane (226.4 mgs, 0.275 mmol). The reaction vessel was sealed and heated to 90° C. for 4 hours. The reaction mixture was cooled to ambient temperature, filtered through GF/F paper, and concentrated. The residue was diluted with EtOAc, washed with water and brine, dried over Na₂SO₄, filtered and concentrated in vacuo to provide tert-butyl 3-(2-fluoroethyl)-3-(4-(4,4,5,5-tetramethyl-1,3,2-dioxaborolan-2-yl)-1H-pyrazol-1-yl)azetidine-1-carboxylate (1088 mg, 2.753 mmol, 100% yield).

Step G: 5-Chloro-7-(1-methyl-1H-pyrazol-4-yl)imidazo[1,2-c]pyrimidine (580.0 mg, 2.482 mmol) was dissolved in 4:1 ACN:water (0.2 M) and treated with tert-butyl 3-(2-fluoroethyl)-3-(4-(4,4,5,5-tetramethyl-1,3,2-dioxaborolan-2-yl)-1H-pyrazol-1-yl)azetidine-1-carboxylate (1079 mg, 2.730 mmol) and potassium carbonate (686.1 mg, 4.965 mmol). The reaction mixture was degassed with argon and to this was added tetrakis(triphenylphosphine)palladium (0) (143.4 mg, 0.124 mmol). The reaction vessel was sealed and heated to 85° C. for 24 hours. The reaction mixture was cooled to ambient temperature and concentrated. The crude product was purified by column chromatography (DCM/IPA with 2% NH₄OH) to provide tert-butyl 3-(2-fluoroethyl)-3-(4-(7-(1-methyl-1H-pyrazol-4-yl)imidazo[1,2-c]pyrimidin-5-yl)-1H-pyrazol-1-yl)azetidine-1-carboxylate (484.2 mg, 1.038 mmol, 41.8% yield). m/z (APCI-pos) M+1=467.2.

Step H: Tert-butyl 3-(2-fluoroethyl)-3-(4-(7-(1-methyl-1H-pyrazol-4-yl)imidazo[1,2-c]pyrimidin-5-yl)-1H-pyrazol-1-yl)azetidine-1-carboxylate (484.2 mg, 1.038 mmol) was dissolved in 4 N HCl in 1,4-dioxane (1.0 mL) and stirred at ambient temperature for 2 hours. The reaction mixture was then concentrated in vacuo to provide 5-(1-(3-2-fluoroethyl)azetidin-3-yl)-1H-pyrazol-4-yl)-7-(1-methyl-1H-pyrazol-4-yl)imidazo[1,2-c]pyrimidine trihydrochloride (493.8 mg, 1.038 mmol, 100% yield). m/z (APCI-pos) M+1=367.1

Step I: 5-(1-(3-(2-Fluoroethyl)azetidin-3-yl)-1H-pyrazol-4-yl)-7-(1-methyl-1H-pyrazol-4-yl)imidazo[1,2-c]pyrimidine trihydrochloride (207.0 mg, 0.435 mmol) was dissolved in DCM (0.1 M) and treated sequentially with N,N-diisopropylethylamine (758 μL, 4.351 mmol), N,N-dimethylpyridin-4-amine (5.3 mg, 0.044 mmol), and trifluoromethanesulfonic anhydride (103 μL, 0.609 mmol) and stirred at ambient temperature for 30 minutes. The mixture was then diluted with additional DCM and washed with NaHCO₃, dried over Na₂SO₄, filtered and concentrated in vacuo. The crude product was purified by silica gel chromatography (DCM/IPA with 2% NH₄OH) followed by C18 chromatography (water/ACN) to provide 5-(1-(3-(2-fluoroethyl)-1-(trifluoromethylsulfonyl)azetidin-3-yl)-1H-pyrazol-4-yl)-7-(1-methyl-1H-pyrazol-4-yl)imidazo[1,2-c]pyrimidine (40.8 mg, 0.082 mmol, 18.8% yield). m/z (APCI-pos) M+1=499.1.

### Example 19

### 5-(1-(3-(2-Fluoroethyl)-1-(2,2,2-trifluoroethyl)azetidin-3-yl)-1H-pyrazol-4-yl)-7-(1-methyl-1H-pyrazol-4-yl)imidazo[1,2-c]pyrimidine

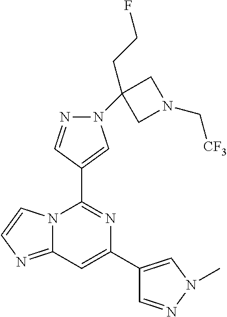 [SMILES: Cn1cc(-c2cc3nccn3c(-c3cnn(C4(CCF)CN(CC(F)(F)F)C4)c3)n2)cn1]

5-(1-(3-(2-Fluoroethyl)azetidin-3-yl)-1H-pyrazol-4-yl)-7-(1-methyl-1H-pyrazol-4-yl)imidazo[1,2-c]pyrimidine trihydrochloride (27.3 mg, 0.057 mmol) was dissolved in DCM (0.1 M) and treated sequentially with N-ethyl-N-isopropylpropan-2-amine (100 μL, 0.574 mmol) and 2,2,2-trifluoroethyl trifluoromethanesulfonate (26.6 mg, 0.115 mmol) and stirred at ambient temperature for 2 hours. The reaction mixture was then diluted with additional DCM and washed with NaHCO₃, dried over Na₂SO₄, filtered and concentrated in vacuo. The crude product was purified by silica gel chromatography (DCM/IPA with 2% NH₄OH) followed by C18 chromatography (water/ACN) to provide 5-(1-(3-(2-fluoroethyl)-1-(2,2,2-trifluoroethyl)azetidin-3-yl)-1H-pyrazol-4-yl)-7-(1-methyl-1H-pyrazol-4-yl)imidazo[1,2-c]pyrimidine (7.2 mg, 0.016 mmol, 28.0% yield). m/z (APCI-pos) M+1=449.2.

### Example 20

### 5-(1-(3-ethyl-1-(trifluoromethylsulfonyl)azetidin-3-yl)-1H-pyrazol-4-yl)-7-(1-methyl-1H-pyrazol-4-yl)imidazo[1,2-c]pyrimidine

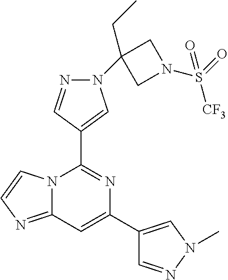 [SMILES: CCC1(n2cc(-c3nc(-c4cnn(C)c4)cc4nccn34)cn2)CN(S(=O)(=O)C(F)(F)F)C1]

Step A: Tert-butyl 3-(4-bromo-1H-pyrazol-1-yl)-3-(2-(methylsulfonyloxy)ethyl)azetidine-1-carboxylate (8.95 g, 21.1 mmol) was dissolved in THF (0.2 M) and treated with tetrabutylammonium fluoride (28.1 mL, 42.2 mmol, 1.5 M) and stirred at 60° C. for 2 hours. The reaction mixture was cooled to ambient temperature and concentrated. The residue was diluted with EtOAc, washed with water and brine, dried over Na₂SO₄, filtered and concentrated in vacuo. The crude product was purified by silica gel chromatography to provide tert-butyl 3-(4-bromo-1H-pyrazol-1-yl)-3-vinylazetidine-1-carboxylate (1.51 g, 4.60 mmol, 21.8% yield).

Step B: Tert-butyl 3-(4-bromo-1H-pyrazol-1-yl)-3-vinylazetidine-1-carboxylate (443.7 mg, 1.352 mmol) was dissolved in dioxane (6.8 mL, 0.2 M) and to this was added bis(pinacolato)diboron (377.6 mg, 1.487 mmol), potassium acetate (398.0 mg, 4.056 mmol) and 1,1′-bis(diphenylphosphino)ferrocene palladium (II) chloride:dichloromethane complex (111.2 mg, 0.1352 mmol). The reaction mixture was degassed with argon for 15 minutes and then heated to 90° C. for 4 hours under argon. The mixture was then cooled to ambient temperature and concentrated. The residue was diluted with EtOAc, washed with water and brine, dried, filtered and concentrated. The crude product was used directly in Step C.

Step C: 5-Chloro-7-(1-methyl-1H-pyrazol-4-yl)imidazo[1,2-c]pyrimidine (285 mg, 1.220 mmol) was dissolved in 4:1 ACN/water (6.1 mL, 0.2 M) and to this was added tert-butyl 3-(4-(4,4,5,5-tetramethyl-1,3,2-dioxaborolan-2-yl)-1H-pyrazol-1-yl)-3-vinylazetidine-1-carboxylate (503.5 mg, 1.342 mmol), potassium carbonate (337.2 mg, 2.439 mmol) and tetrakis(triphenylphosphine)palladium (0) (70.47 mg, 0.0610 mmol). The reaction mixture was degassed with argon for 15 minutes and then heated to 85° C. for 24 hours under argon. The mixture was then cooled to ambient temperature and concentrated. Silica gel chromatography provided tert-butyl 3-(4-(7-(1-methyl-1H-pyrazol-4-yl)imidazo[1,2-c]pyrimidin-5-yl)-1H-pyrazol-1-yl)-3-vinylazetidine-1-carboxylate (0.219 g, 0.49 mmol, 40% yield over 2 steps). m/z (APCI-pos) M+1=447.2.

Step D: To a solution of tert-butyl 3-(4-(7-(1-methyl-1H-pyrazol-4-yl)imidazo[1,2-c]pyrimidin-5-yl)-1H-pyrazol-1-yl)-3-vinylazetidine-1-carboxylate (0.219 g, 0.490 mmol) in EtOAc/MeOH (1:1) was added 10% Pd/C (0.0522 g, 0.0490 mmol) and the mixture was purged with nitrogen for 10 minutes. The reaction mixture was then place under a hydrogen balloon and stirred for 12 hours. The mixture was filtered through GF/F paper to remove palladium and the filtrate was concentrated. The crude product was purified via column chromatography, eluting with EtOAc and then EtOAc/MeOH (20:1) to give tert-butyl 3-ethyl-3-(4-(7-(1-methyl-1H-pyrazol-4-yl)imidazo[1,2-c]pyrimidin-5-yl)-1H-pyrazol-1-yl)azetidine-1-carboxylate (120 mg, 0.268 mmol, 55.0% yield). m/z (APCI-pos) M+1=449.2.

Step E: tert-butyl 3-ethyl-3-(4-(7-(1-methyl-1H-pyrazol-4-yl)imidazo[1,2-c]pyrimidin-5-yl)-1H-pyrazol-1-yl)azetidine-1-carboxylate (0.120 g, 0.268 mmol) in MeOH was treated with 4N HCl in dioxane (5.0 mL, 20 mmol) at room temperature for 3 hours. The reaction mixture was concentrated to give 5-(1-(3-Ethylazetidin-3-yl)-1H-pyrazol-4-yl)-7-(1-methyl-1H-pyrazol-4-yl)imidazo[1,2-c]pyrimidine trihydrochloride, which was used without purification in Step F.

Step F: 5-(1-(3-ethylazetidin-3-yl)-1H-pyrazol-4-yl)-7-(1-methyl-1H-pyrazol-4-yl)imidazo[1,2-c]pyrimidine trihydrochloride (0.060 g, 0.1311 mmol) was dissolved in DCM (5 mL) and treated with N,N-diisopropylethylamine (0.2283 mL, 1.311 mmol), N,N-dimethylpyridin-4-amine (0.001601 g, 0.01311 mmol), and then trifluoromethanesulfonic anhydride (0.03087 mL, 0.1835 mmol) at ambient temperature for 1 hour. The reaction mixture was diluted with DCM and washed with water. The combined organic layers were dried, filtered and concentrated. The crude product was purified via column chromatography, eluting with EtOAc and then EtOAc/MeOH (20:1) to give 5-(1-(3-ethyl-1-(trifluoromethylsulfonyl)azetidin-3-yl)-1H-pyrazol-4-yl)-7-(1-methyl-1H-pyrazol-4-yl)imidazo[1,2-c]pyrimidine (56 mg, 0.117 mmol, 89.0% yield). m/z (APCI-pos) M+1=481.1.

### Example 21

### 7-(1-methyl-1H-pyrazol-4-yl)-5-(1-(2-(methylthio)ethyl)-1H-pyrazol-4-yl)imidazo[1,2-c]pyrimidine

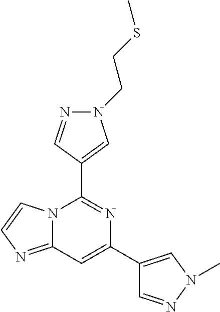 [SMILES: CSCCn1cc(-c2nc(-c3cnn(C)c3)cc3nccn23)cn1]

### Step A Preparation of 7-(1-methyl-1H-pyrazol-4-yl)imidazo[1,2-c]pyrimidin-5(6H)-one

To a mixture of 7-chloroimidazo[1,2-c]pyrimidin-5(6H)-one (Preparation E; 10.0 g, 59.0 mmol), 1-methyl-4-(4,4,5,5-tetramethyl-1,3,2-dioxaborolan-2-yl)-1H-pyrazole (19.0 g, 88.5 mmol) and XPHOS (2.81 g, 5.90 mmol) in isopropyl alcohol (400 mL) was added 2M K₃PO₄ (88.5 mL, 177 mmol). The mixture was purged with N₂ for 15 minutes with vigorous mixing and Pd₂dba₃ (2.70 g, 2.95 mmol) was added. The mixture was heated at reflux under a N₂ atmosphere for 20 hours. The mixture was charged additional 1-methyl-4-(4,4,5,5-tetramethyl-1,3,2-dioxaborolan-2-yl)-1H-pyrazole (6.00 g) and Pd₂dba₃ (1.00 g) and heated at reflux for an additional 20 hours. The mixture was cooled to ambient temperature and concentrated to an aqueous syrup. The syrup was partitioned into H₂O (500 mL) and 50% EtOAc-hexanes (250 mL) and mixed. The mixture was filtered through filter paper and the orange organic layer was removed. The aqueous layer was washed with 50% EtOAc/hexanes and was cooled on an ice bath. The solution was treated with concentrated HCl to pH 6 with stirring and the resulting fine precipitate was collected, washed with H₂O and Et₂O and dried under vacuum to provide the title compound (9.65 g, 76% yield) as faint grey solid. MS (apci) m/z=216.2 (M+H).

### Step B: Preparation of 5-chloro-7-(1-methyl-1H-pyrazol-4-yl)imidazo[1,2-c]pyrimidine

To a suspension of 7-(1-methyl-1H-pyrazol-4-yl)imidazo[1,2-c]pyrimidin-5(6H)-one (9.60 g, 44.6 mmol) in dry DCM (90 mL) was added DIEA and the suspension stirred at ambient temperature for 5 minutes. The mixture was cooled to 0° C. and POCl₃ (12.3 mL, 134 mmol) was added over 5 minutes. The mixture was allowed to reach ambient temperature and the resulting thick slurry was treated with dry DCM (50 mL). The mixture was vigorously stirred at ambient temperature for 23 hours. The resulting light tan suspension was diluted with hexanes (90 mL) and collected by vacuum filtration. The collected solid was washed with Et₂O and dried in vacuum to give the crude product salt. The salt was suspended in 5:20:75 MeOH/DIEA/EtOAc (200 mL) and stirred for 30 minutes at ambient temperature. The mixture was filtered through a SiO₂ plug capped with a Celite layer eluting with 5% MeOH/EtOAc. The filtrate was concentrated and the residual solid dried in vacuum to provide 5-chloro-7-(1-methyl-1H-pyrazol-4-yl)imidazo[1,2-c]pyrimidine (5.65 g, 54% yield) as a light cream colored solid. MS (apci) m/z=234.2 (M+H).

Step C: In a 5 L 4-necked flask with an overhead mechanical stirrer was added 5-chloro-7-(1-methyl-1H-pyrazol-4-yl)imidazo[1,2-c]pyrimidine (34.83 g, 149.1 mmol), tert-butyl 3-(cyanomethyl)-3-(4-(4,4,5,5-tetramethyl-1,3,2-dioxaborolan-2-yl)-1H-pyrazol-1-yl)azetidine-1-carboxylate (Preparation D; 86.82 g, 223.6 mmol), and K₃PO₄ (94.92 g, 447.2 mmol) by powder funnel. Dioxane (745.3 mL, 149.1 mmol) was added to rinse down funnel. Pd(PPh₃)₄ (17.23 g, 14.91 mmol) was added, followed by 74.5 mL of water. The reaction mixture was slowly heated to 70° C. as measured by an internal temperature probe. After heating for 6 hours, the reaction mixture was cooled to ambient temperature. The reaction mixture was diluted in EtOAc (500 mL) and water (100 mL), and then the resultant solids were filtered out. The solids were washed with EtOAc (2×500 mL) to afford a grey-white solid, which was re-introduced back to the 5 L 4-neck flask and diluted with 1 L of water and 300 mL of EtOAc. This was stirred for 3 hours, and then the solids were isolated by filtration. After washing with EtOAc (2×500 mL), the solids were dried to afford tert-butyl 3-(cyanomethyl)-3-(4-(7-(1-methyl-1H-pyrazol-4-yl)imidazo[1,2-c]pyrimidin-5-yl)-1H-pyrazol-1-yl)azetidine-1-carboxylate (60.83 g, 132.4 mmol, 88.81% yield). MS (apci) m/z=460.1 (M+H).

Step D: A 5 L 4-neck flask was equipped with an overhead stirrer and purged with N₂. To this was added tert-butyl 3-(cyanomethyl)-3-(4-(7-(1-methyl-1H-pyrazol-4-yl)imidazo[1,2-c]pyrimidin-5-yl)-1H-pyrazol-1-yl)azetidine-1-carboxylate (Example 61; 60.83 g, 132.4 mmol) and dioxane (661.9 mL, 132.4 mmol) and the flask was placed in a cool water bath. 4N HCl in dioxane (661.9 mL, 2648 mmol) was added in a fast stream. An additional 50 mL of dioxane was added to wash down the sides. The reaction stalled after 2 hours, so another 140 mL of HCl in dioxane was added. After 4 hours, another 50 mL of HCl in dioxane was added to drive to completion. The solids were filtered, washed with dioxane, and then washed with Et₂O. The resultant solids were dried under high vacuum to afford 76 g (77% by weight, 103% yield) of 2-(3-(4-(7-(1-methyl-1H-pyrazol-4-yl)imidazo[1,2-c]pyrimidin-5-yl)-1H-pyrazol-1-yl)azetidin-3-yl)acetonitrile trihydrochloride as a powdery white solid. MS (apci) m/z=360.2 (M+H).

Step E: To a solution of 2-(3-(4-(7-(1-methyl-1H-pyrazol-4-yl)imidazo[1,2-c]pyrimidin-5-yl)-1H-pyrazol-1-yl)azetidin-3-yl)acetonitrile trihydrochloride (0.100 g, 0.213 mmol) in DMF (1 mL) at 0° C. was added NaH (0.034 g, 0.85 mmol). The resulting mixture was stirred at 0° C. for 15 minutes. To this mixture was added a solution of 2-chloroethyl methyl sulfide (0.023 mL, 0.23 mmol) in DMF (0.5 mL). The reaction mixture was warmed to ambient temperature and stirred for 24 hours. After addition of additional 0.01 mL of 2-chloroethyl methyl sulfide, the reaction mixture was stirred for additional 21 hours. The reaction mixture was cooled to 0° C. and quenched with saturated aqueous NH₄Cl. The resulting mixture was extracted with CH₂Cl₂ three times. The combined organic layers were dried over MgSO₄, filtered, and concentrated under reduced pressure to give the crude material that was purified by silica gel flash column chromatography (CH₂Cl₂ to 5% MeOH in CH₂Cl₂). 7-(1-methyl-1H-pyrazol-4-yl)-5-(1-(2-(methylthio)ethyl)-1H-pyrazol-4-yl)imidazo[1,2-c]pyrimidine was isolated as a by-product of the reaction (0.034 g, 0.100 mmol, 47.0% yield). LCMS (APCI) M+1=340.1.

The following compounds were also prepared according to above described methods.

TABLE B

| Ex. # | Structure | Name | Data |
|---|---|---|---|
| 22 | 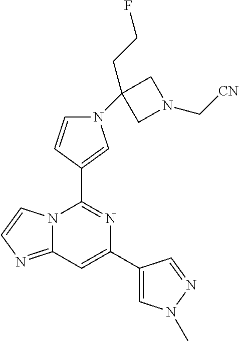 [SMILES: Cn1cc(-c2cc3nccn3c(-c3ccn(C4(CCF)CN(CC#N)C4)c3)n2)cn1] | 2-(3-(2-fluoroethyl)-3-(3-(7-(1- methyl-1H-pyrazol-4- yl)imidazo[1,2-c]pyrimidin-5-yl)- 1H-pyrrol-1-yl)azetidin-1- yl)acetonitrile | ES + APCI [M + 1] 405.3 |
| 23 | 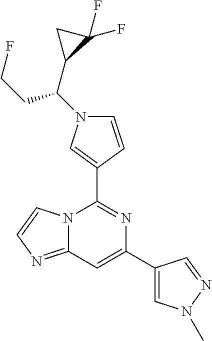 [SMILES: Cn1cc(-c2cc3nccn3c(-c3ccn([C@H](CCF)[C@H]4CC4(F)F)c3)n2)cn1] | 5-(1-((R)-1-((R)-2,2- difluorocyclopropyl)-3- fluoropropyl)-1H-pyrrol-3-yl)-7- (1-methyl-1H-pyrazol-4- yl)imidazo[1,2-c]pyrimidine | m/z ES + APCI [M + 1] 402.1 |
| 24 | 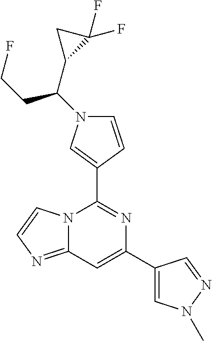 [SMILES: Cn1cc(-c2cc3nccn3c(-c3ccn([C@@H](CCF)[C@@H]4CC4(F)F)c3)n2)cn1] | 5-(1-((S)-1-((S)-2,2- difluorocyclopropyl)-3- fluoropropyl)-1H-pyrrol-3-yl)-7- (1-methyl-1H-pyrazol-4- yl)imidazo[1,2-c]pyrimidine | m/z ES + APCI [M + 1] 402.1 |
| 25 | 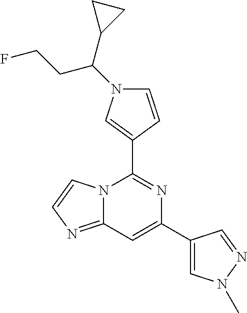 [SMILES: Cn1cc(-c2cc3nccn3c(-c3ccn(C(CCF)C4CC4)c3)n2)cn1] | 5-(1-(1-cyclopropyl-3- fluoropropyl)-1H-pyrrol-3-yl)-7- (1-methyl-1H-pyrazol-4- yl)imidazo[1,2-c]pyrimidine | m/z ES + APCI [M + 1] 365.2 |
| 26 | 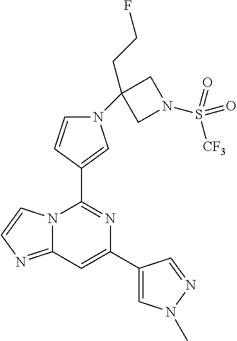 [SMILES: Cn1cc(-c2cc3nccn3c(-c3ccn(C4(CCF)CN(S(=O)(=O)C(F)(F)F)C4)c3)n2)cn1] | 5-(1-(3-(2-fluoroethyl)-1- ((trifluoromethyl)sulfonyl)azetidin- 3-yl)-1H-pyrrol-3-yl)-7-(1-methyl- 1H-pyrazol-4-yl)imidazo[1,2- c]pyrimidine | ES + APCI [M + 1] 498.2 |
| 27 | 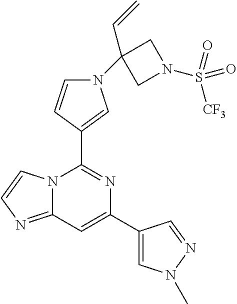 [SMILES: C=CC1(n2ccc(-c3nc(-c4cnn(C)c4)cc4nccn34)c2)CN(S(=O)(=O)C(F)(F)F)C1] | 7-(1-methyl-1H-pyrazol-4-yl)-5- (1-(1-((trifluoromethyl)sulfonyl)-3- vinylazetidin-3-yl)-1H-pyrrol-3- yl)imidazo[1,2-c]pyrimidine | ES + APCI [M + 1] 478.2 |
| 28 | 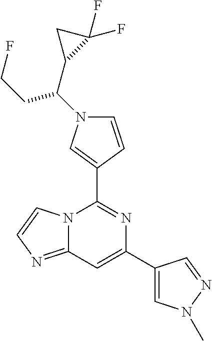 [SMILES: Cn1cc(-c2cc3nccn3c(-c3ccn([C@H](CCF)[C@@H]4CC4(F)F)c3)n2)cn1] | 5-(1-((R)-1-((S)-2,2- difluorocyclopropyl)-3- fluoropropyl)-1H-pyrrol-3-yl)-7- (1-methyl-1H-pyrazol-4- yl)imidazo[1,2-c]pyrimidine | m/z ES + APCI [M + 1] 401.1 |
| 29 | 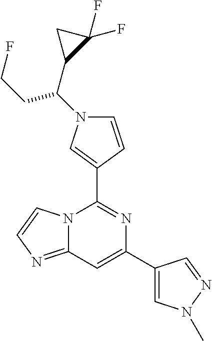 [SMILES: Cn1cc(-c2cc3nccn3c(-c3ccn([C@H](CCF)[C@H]4CC4(F)F)c3)n2)cn1] | 5-(1-((R)-1-((R)-2,2- difluorocyclopropyl)-3- fluoropropyl)-1H-pyrrol-3-yl)-7- (1-methyl-1H-pyrazol-4- yl)imidazo[1,2-c]pyrimidine | m/z ES + APCI [M + 1] 401.1 |
| 30 | 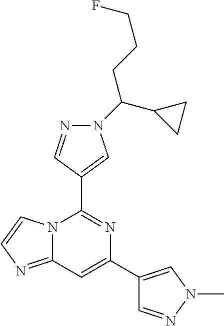 [SMILES: Cn1cc(-c2cc3nccn3c(-c3cnn(C(CCCF)C4CC4)c3)n2)cn1] | 5-(1-(1-cyclopropyl-4- fluorobutyl)-1H-pyrazol-4-yl)-7- (1-methyl-1H-pyrazol-4- yl)imidazo[1,2-c]pyrimidine | ES + APCI [M + 1] 380.2 |
| 31 | 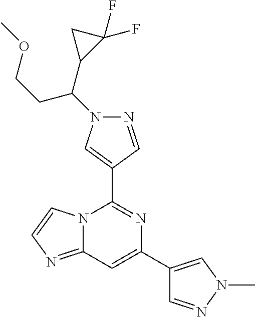 | 5-(1-(1-2,2-difluorocyclopropyl)- 3-methoxypropyl)-1H-pyrazol-4- yl)-7-(1-methyl-1H-pyrazol-4- yl)imidazo[1,2-c]pyrimidine | m/z ES + APCI [M + 1] 414.2 |
| 32 | 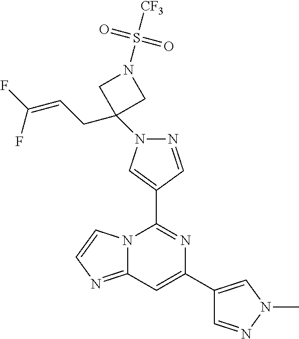 [SMILES: Cn1cc(-c2cc3nccn3c(-c3cnn(C4(CC=C(F)F)CN(S(=O)(=O)C(F)(F)F)C4)c3)n2)cn1] | 5-(1-(3-(3,3-difluoroallyl)-1- ((trifluoromethyl)sulfonyl)azetidin- 3-yl)-1H-pyrazol-4-yl)-7-(1- methyl-1H-pyrazol-4- yl)imidazo[1,2-c]pyrimidine | ES + APCI [M + 1] 529.1 |
| 33 | 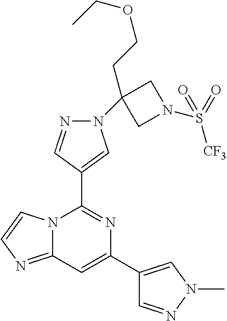 [SMILES: CCOCCC1(n2cc(-c3nc(-c4cnn(C)c4)cc4nccn34)cn2)CN(S(=O)(=O)C(F)(F)F)C1] | 5-(1-(3-(2-ethoxyethyl)-1- ((trifluoromethyl)sulfonyl)azetidin- 3-yl)-1H-pyrazol-4-yl)-7-(1- methyl-1H-pyrazol-4- yl)imidazo[1,2-c]pyrimidine | ES + APCI [M + 1] 525.3 |
| 34 | 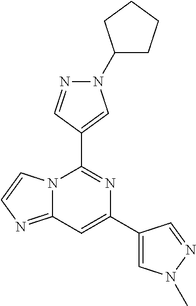 [SMILES: Cn1cc(-c2cc3nccn3c(-c3cnn(C4CCCC4)c3)n2)cn1] | 5-(1-cyclopentyl-1H-pyrazol-4-yl)- 7-(1-methyl-1H-pyrazol-4- yl)imidazo[1,2-c]pyrimidine | m/z ES + APCI [M + 1] 334.2 |
| 35 | 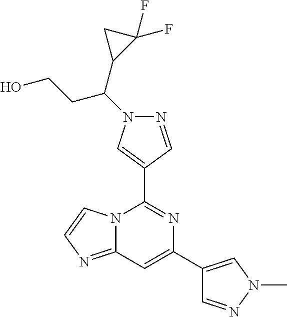 [SMILES: Cn1cc(-c2cc3nccn3c(-c3cnn(C(CCO)C4CC4(F)F)c3)n2)cn1] | 3-(2,2-difluorocyclopropyl)-3-(4- (7-(1-methyl-1H-pyrazol-4- yl)imidazo[1,2-c]pyrimidin-5-yl)- 1H-pyrazol-1-yl)propan-1-ol | m/z ES + APCI [M + 1] 400.1 |
| 36 | 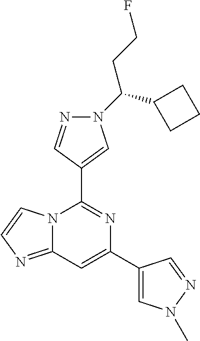 [SMILES: Cn1cc(-c2cc3nccn3c(-c3cnn([C@H](CCF)C4CCC4)c3)n2)cn1] | (R)-5-(1-(1-cyclobutyl-3- fluoropropyl)-1H-pyrazol-4-yl)-7- (1-methyl-1H-pyrazol-4- yl)imidazo[1,2-c]pyrimidine | ES + APCI [M + 1] 380.2 |
| 37 | 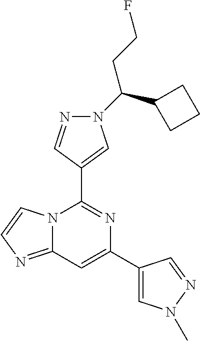 [SMILES: Cn1cc(-c2cc3nccn3c(-c3cnn([C@@H](CCF)C4CCC4)c3)n2)cn1] | (S)-5-(1-(1-cyclobutyl-3- fluoropropyl)-1H-pyrazol-4-yl)-7- (1-methyl-1H-pyrazol-4- yl)imidazo[1,2-c]pyrimidine | ES + APCI [M + 1] 380.1 |
| 38 | 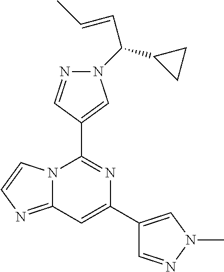 [SMILES: C/C=C/[C@H](C1CC1)n1cc(-c2nc(-c3cnn(C)c3)cc3nccn23)cn1] | (S,E)-5-(1-(1-cyclopropylbut-2-en- 1-yl)-1H-pyrazol-4-yl)-7-(1- methyl-1H-pyrazol-4- yl)imidazo[1,2-c]pyrimidine | m/z ES + APCI [M + 1] 360.1 |
| 39 | 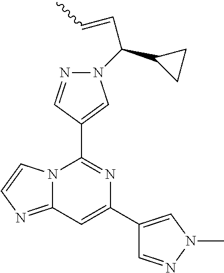 | (R)-5-(1-(1-cyclopropylbut-2-en-1- yl)-1H-pyrazol-4-yl)-7-(1-methyl- 1H-pyrazol-4-yl)imidazo[1,2- c]pyrimidine | m/z ES + APCI [M + 1] 360.1 |
| 40 | 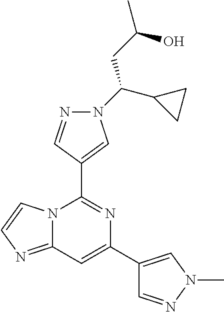 [SMILES: C[C@@H](O)C[C@H](C1CC1)n1cc(-c2nc(-c3cnn(C)c3)cc3nccn23)cn1] | (2R,4R)-4-cyclopropyl-4-(4-(7-(1- methyl-1H-pyrazol-4- yl)imidazo[1,2-c]pyrimidin-5-yl)- 1H-pyrazol-1-yl)butan-2-ol | m/z ES + APCI [M + 1] 378.2 |
| 41 | 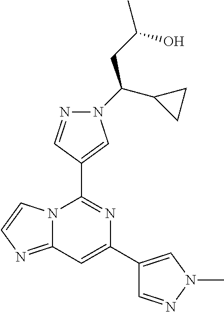 [SMILES: C[C@H](O)C[C@@H](C1CC1)n1cc(-c2nc(-c3cnn(C)c3)cc3nccn23)cn1] | (2S,4S)-4-cyclopropyl-4-(4-(7-(1- methyl-1H-pyrazol-4- yl)imidazo[1,2-c]pyrimidin-5-yl)- 1H-pyrazol-1-yl)butan-2-ol | m/z ES + APCI [M + 1] 378.2 |
| 42 | 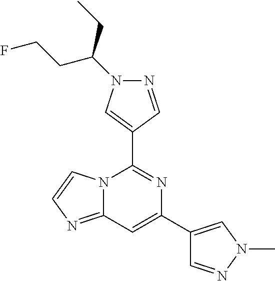 [SMILES: CC[C@H](CCF)n1cc(-c2nc(-c3cnn(C)c3)cc3nccn23)cn1] | (R)-5-(1-(1-fluoropentan-3-yl)-1H- pyrazol-4-yl)-7-(1-methyl-1H- pyrazol-4-yl)imidazo[1,2- c]pyrimidine | m/z ES + APCI [M + 1] 354.5 |
| 43 | 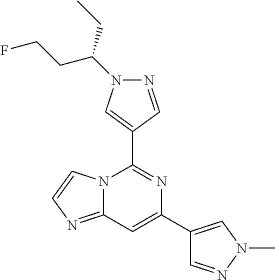 [SMILES: CC[C@@H](CCF)n1cc(-c2nc(-c3cnn(C)c3)cc3nccn23)cn1] | (S)-5-(1-(1-fluoropentan-3-yl)-1H- pyrazol-4-yl)-7-(1-methyl-1H- pyrazol-4-yl)imidazo[1,2- c]pyrimidine | m/z ES + APCI [M + 1] 354.5 |
| 44 | 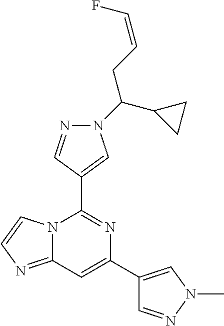 [SMILES: Cn1cc(-c2cc3nccn3c(-c3cnn(C(C/C=C\F)C4CC4)c3)n2)cn1] | (Z)-5-(1-(1-cyclopropyl-4- fluorobut-3-en-1-yl)-1H-pyrazol-4- yl)-7-(1-methyl-1H-pyrazol-4- yl)imidazo[1,2-c]pyrimidine | m/z ES + APCI [M + 1] 378.2 |
| 45 | 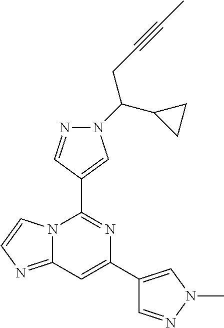 [SMILES: CC#CCC(C1CC1)n1cc(-c2nc(-c3cnn(C)c3)cc3nccn23)cn1] | 5-(1-(1-cyclopropylpent-3-yn-1- yl)-1H-pyrazol-4-yl)-7-(1-methyl- 1H-pyrazol-4-yl)imidazo[1,2- c]pyrimidine | m/z ES + APCI [M + 1] 372.2 |
| 46 | 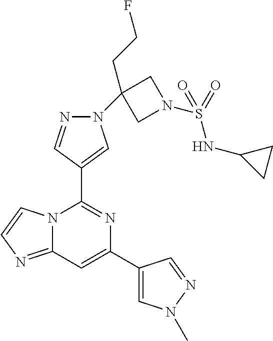 [SMILES: Cn1cc(-c2cc3nccn3c(-c3cnn(C4(CCF)CN(S(=O)(=O)NC5CC5)C4)c3)n2)cn1] | N-cyclopropyl-3-(2-fluoroethyl)-3- (4-(7-(1-methyl-1H-pyrazol-4- yl)imidazo[l,2-c]pyrimidin-5-yl)- 1H-pyrazol-1-yl)azetidine-1- sulfonamide | m/z ES + APCI [M + 1] 486.2 |
| 47 | 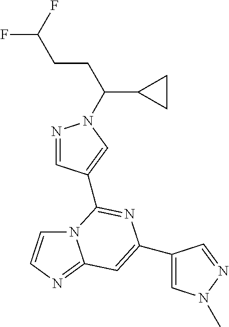 [SMILES: Cn1cc(-c2cc3nccn3c(-c3cnn(C(CCC(F)F)C4CC4)c3)n2)cn1] | 5-(1-(1-cyclopropyl-4,4- difluorobutyl)-1H-pyrazol-4-yl)-7- (1-methyl-1H-pyrazol-4- yl)imidazo[1,2-c]pyrimidine | ES + APCI [M + 1] 398.1 |
| 48 | 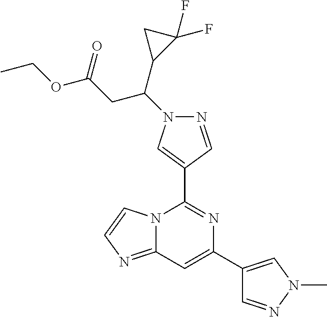 [SMILES: CCOC(=O)CC(C1CC1(F)F)n1cc(-c2nc(-c3cnn(C)c3)cc3nccn23)cn1] | ethyl 3-(2,2-difluorocyclopropyl)- 3-(4-(7-(1-methyl-1H-pyrazol-4- yl)imidazo[1,2-c]pyrimidin-5-yl)- 1H-pyrazol-1-yl)propanoate | m/z ES + APCI [M + 1] 442.1 |
| 49 | 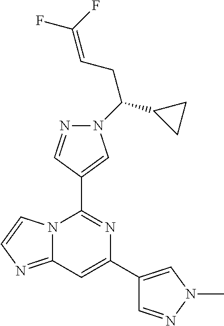 [SMILES: Cn1cc(-c2cc3nccn3c(-c3cnn([C@H](CC=C(F)F)C4CC4)c3)n2)cn1] | (R)-5-(1-(1-cyclopropyl-4,4- difluorobut-3-en-1-yl)-1H-pyrazol- 4-yl)-7-(1-methyl-1H-pyrazol-4- yl)imidazo[1,2-c]pyrimidine | m/z ES + APCI [M + 1] 396.1 |
| 50 | 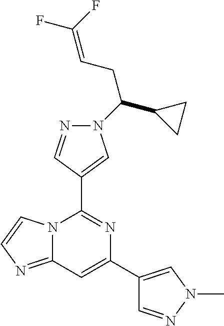 [SMILES: Cn1cc(-c2cc3nccn3c(-c3cnn([C@@H](CC=C(F)F)C4CC4)c3)n2)cn1] | (S)-5-(1-(1-cyclopropyl-4,4- difluorobut-3-en-1-yl)-1H-pyrazol- 4-yl)-7-(1-methyl-1H-pyrazol-4- yl)imidazo[1,2-c]pyrimidine | m/z ES + APCI [M + 1] 396.1 |
| 51 | 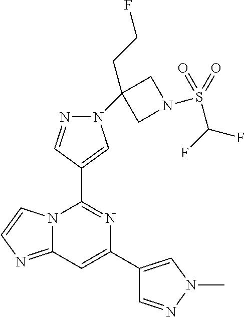 [SMILES: Cn1cc(-c2cc3nccn3c(-c3cnn(C4(CCF)CN(S(=O)(=O)C(F)F)C4)c3)n2)cn1] | 5-(1-(1-((difluoromethyl)sulfonyl)- 3-(2-fluoroethyl)azetidin-3-yl)-1H- pyrazol-4-yl-7-(1-methyl-1H- pyrazol-4-yl)imidazo[1,2- c]pyrimidine | m/z ES + APCI [M + 1] 481.2 |
| 52 | 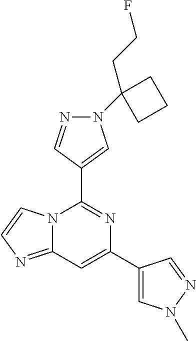 [SMILES: Cn1cc(-c2cc3nccn3c(-c3cnn(C4(CCF)CCC4)c3)n2)cn1] | 5-(1-(1-(2-fluoroethyl)cyclobutyl)- 1H-pyrazol-4-yl)-7-(1-methyl-1H- pyrazol-4-yl)imidazo[1,2- c]pyrimidine | ES + APCI [M + 1] 366.1 |
| 53 | 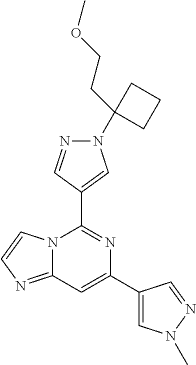 [SMILES: COCCC1(n2cc(-c3nc(-c4cnn(C)c4)cc4nccn34)cn2)CCC1] | 5-(1-(1-(2- methoxyethyl)cyclobutyl)-1H- pyrazol-4-yl)-7-(1-methyl-1H- pyrazol-4-yl)imidazo[1,2- c]pyrimidine | ES + APCI [M + 1] 378.2 |
| 54 | 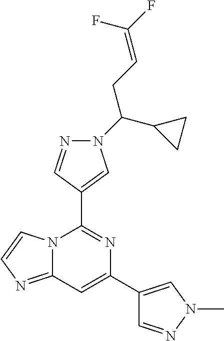 [SMILES: Cn1cc(-c2cc3nccn3c(-c3cnn(C(CC=C(F)F)C4CC4)c3)n2)cn1] | 5-(1-(1-cyclopropyl-4,4- difluorobut-3-en-1-yl)-1H-pyrazol- 4-yl)-7-(1-methyl-1H-pyrazol-4- yl)imidazo[1,2-c]pyrimidine | ES + APCI [M + 1] 396.1 |
| 55 | 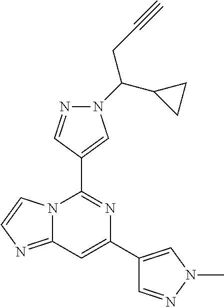 [SMILES: C#CCC(C1CC1)n1cc(-c2nc(-c3cnn(C)c3)cc3nccn23)cn1] | 5-(1-(1-cyclopropylbut-3-yn-1-yl)- 1H-pyrazol-4-yl)-7-(1-methyl-1H- pyrazol-4-yl)imidazo[1,2- c]pyrimidine | ES + APCI [M + 1] 358.1 |
| 56 | 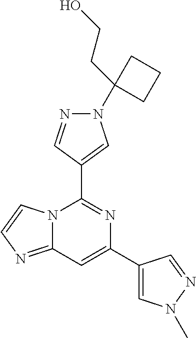 [SMILES: Cn1cc(-c2cc3nccn3c(-c3cnn(C4(CCO)CCC4)c3)n2)cn1] | 2-(1-(4-(7-(1-methyl-1H-pyrazol- 4-yl)imidazo[1,2-c]pyrimidin-5- yl)-1H-pyrazol-1- yl)cyclobutyl)ethanol | ES + APCI [M + 1] 364.1 |
| 57 | 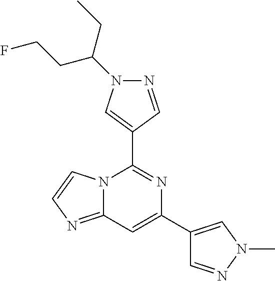 [SMILES: CCC(CCF)n1cc(-c2nc(-c3cnn(C)c3)cc3nccn23)cn1] | 5-(1-(1-fluoropentan-3-yl)-1H- pyrazol-4-yl)-7-(1-methyl-1H- pyrazol-4-yl)imidazo[1,2- c]pyrimidine | m/z ES + APCI [M + 1] 354.2 |
| 58 | 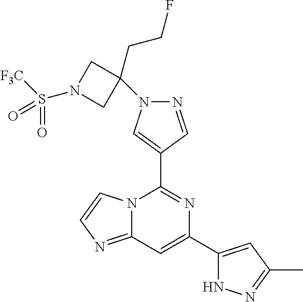 [SMILES: Cc1cc(-c2cc3nccn3c(-c3cnn(C4(CCF)CN(S(=O)(=O)C(F)(F)F)C4)c3)n2)[nH]n1] | 5-(1-(3-(2-fluoroethyl)-1- ((trifluoromethyl)sulfonyl)azetidin- 3-yl)-1H-pyrazol-4-yl)-7-(3- methyl-1H-pyrazol-5- yl)imidazo[1,2-c]pyrimidine | ES + APCI [M + 1] 499.1 |
| 59 | 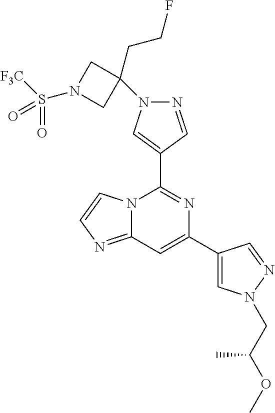 [SMILES: CO[C@H](C)Cn1cc(-c2cc3nccn3c(-c3cnn(C4(CCF)CN(S(=O)(=O)C(F)(F)F)C4)c3)n2)cn1] | (R)-5-(1-(3-(2-fluoroethyl)-1- ((trifluoromethyl)sulfonyl)azetidin- 3-yl)-1H-pyrazol-4-yl)-7-(1-(2- methoxypropyl)-1H-pyrazol-4- yl)imidazo[1,2-c]pyrimidine | ES + APCI [M + 1] 557.1 |
| 60 | 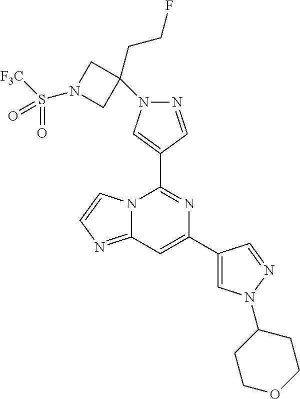 [SMILES: O=S(=O)(N1CC(CCF)(n2cc(-c3nc(-c4cnn(C5CCOCC5)c4)cc4nccn34)cn2)C1)C(F)(F)F] | 5-(1-(3-(2-fluoroethyl)-1- ((trifluoromethyl)sulfonyl)azetidin- 3-yl)-1H-pyrazol-4-yl)-7-(1- (tetrahydro-2H-pyran-4-yl)-1H- pyrazol-4-yl)imidazo[1,2- c]pyrimidine | ES + APCI [M + 1] 569.1 |
| 61 | 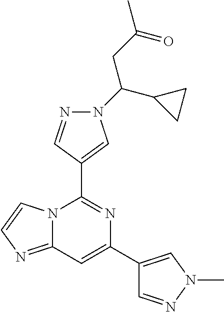 [SMILES: CC(=O)CC(C1CC1)n1cc(-c2nc(-c3cnn(C)c3)cc3nccn23)cn1] | 4-cyclopropyl-4-(4-(7-(1-methyl- 1H-pyrazol-4-yl)imidazo[1,2- c]pyrimidin-5-yl)-1H-pyrazol-1- yl)butan-2-one | m/z ES + APCI [M + 1] 376.2 |
| 62 | 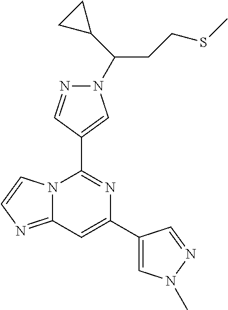 [SMILES: CSCCC(C1CC1)n1cc(-c2nc(-c3cnn(C)c3)cc3nccn23)cn1] | 5-(1-(1-cyclopropyl-3- (methylthio)propyl)-1H-pyrazol-4- yl)-7-(1-methyl-1H-pyrazol-4- yl)imidazo[1,2-c]pyrimidine | m/z ES + APCI [M + 1] 394.2 |
| 63 | 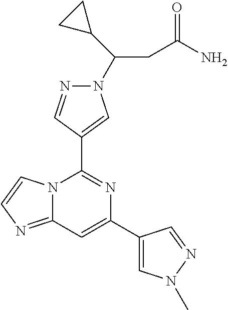 [SMILES: Cn1cc(-c2cc3nccn3c(-c3cnn(C(CC(N)=O)C4CC4)c3)n2)cn1] | 3-cyclopropyl-3-(4-(7-(1-methyl- 1H-pyrazol-4-yl)imidazo[1,2- c]pyrimidin-5-yl)-1H-pyrazol-1- yl)propanamide | m/z ES + APCI [M + 1] 377.2 |
| 64 | 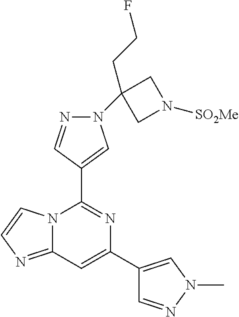 [SMILES: Cn1cc(-c2cc3nccn3c(-c3cnn(C4(CCF)CN(S(C)(=O)=O)C4)c3)n2)cn1] | 5-(1-(3-(2-fluoroethyl)-1- (methylsulfonyl)azetidin-3-yl)-1H- pyrazol-4-yl)-7-(1-methyl-1H- pyrazol-4-yl)imidazo[1,2- c]pyrimidine | m/z ES + APCI [M + 1] 445.2 |
| 65 | 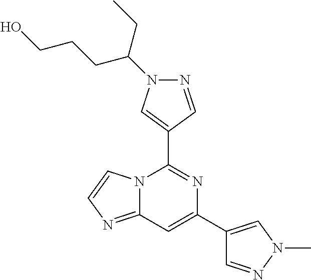 [SMILES: CCC(CCCO)n1cc(-c2nc(-c3cnn(C)c3)cc3nccn23)cn1] | 4-(4-(7-(1-methyl-1H-pyrazol-4- yl)imidazo[1,2-c]pyrimidin-5-yl)- 1H-pyrazol-1-yl)hexan-1-ol | ES + APCI [M + 1] 366.2 |
| 66 | 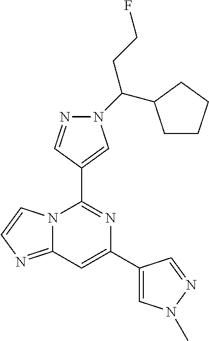 [SMILES: Cn1cc(-c2cc3nccn3c(-c3cnn(C(CCF)C4CCCC4)c3)n2)cn1] | 5-(1-(1-cyclopentyl-3- fluoropropyl)-1H-pyrazol-4-yl)-7- (1-methyl-1H-pyrazol-4- yl)imidazo[1,2-c]pyrimidine | ES + APCI [M + 1] 394.2 |
| 67 | 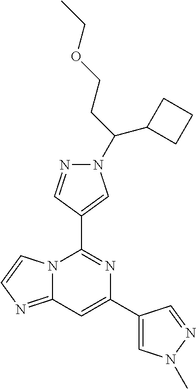 [SMILES: CCOCCC(C1CCC1)n1cc(-c2nc(-c3cnn(C)c3)cc3nccn23)cn1] | 5-(1-(1-cyclobutyl-3- ethoxypropyl)-1H-pyrazol-4-yl)-7- (1-methyl-1H-pyrazol-4- yl)imidazo[1,2-c]pyrimidine | ES + APCI [M + 1] 406.2 |
| 68 | 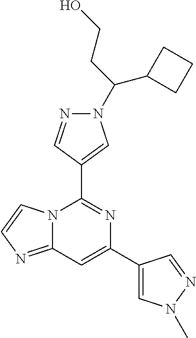 [SMILES: Cn1cc(-c2cc3nccn3c(-c3cnn(C(CCO)C4CCC4)c3)n2)cn1] | 3-cyclobutyl-3-(4-(7-(1-methyl- 1H-pyrazol-4-yl)imidazo[1,2- c]pyrimidin-5-yl)-1H-pyrazol-1- yl)propan-1-ol | ES + APCI [M + 1] 378.2 |
| 69 | 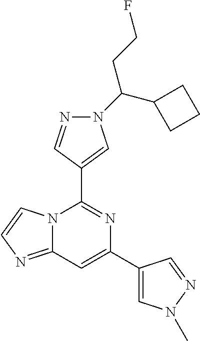 [SMILES: Cn1cc(-c2cc3nccn3c(-c3cnn(C(CCF)C4CCC4)c3)n2)cn1] | 5-(1-(1-cyclobutyl-3- fluoropropyl)-1H-pyrazol-4-yl)-7- (1-methyl-1H-pyrazol-4- yl)imidazo[1,2-c]pyrimidine | ES + APCI [M + 1] 380.1 |
| 70 | 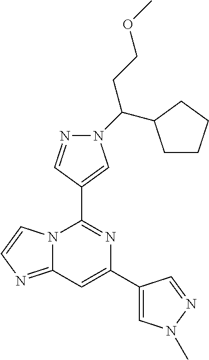 [SMILES: COCCC(C1CCCC1)n1cc(-c2nc(-c3cnn(C)c3)cc3nccn23)cn1] | 5-(1-(1-cyclopentyl-3- methoxypropyl)-1H-pyrazol-4-yl)- 7-(1-methyl-1H-pyrazol-4- yl)imidazo[1,2-c]pyrimidine | ES + APCI [M + 1] 406.2 |
| 71 | 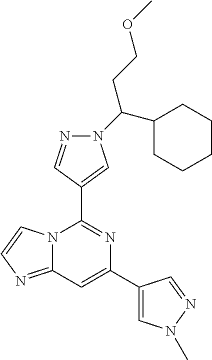 [SMILES: COCCC(C1CCCCC1)n1cc(-c2nc(-c3cnn(C)c3)cc3nccn23)cn1] | 5-(1-(1-cyclohexyl-3- methoxypropyl)-1H-pyrazol-4-yl)- 7-(1-methyl-1H-pyrazol-4- yl)imidazo[1,2-c]pyrimidine | ES + APCI [M + 1] 420.2 |
| 72 | 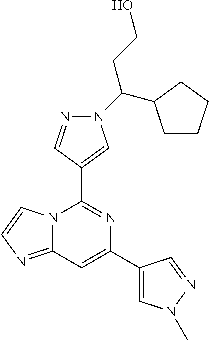 [SMILES: Cn1cc(-c2cc3nccn3c(-c3cnn(C(CCO)C4CCCC4)c3)n2)cn1] | 3-cyclopentyl-3-(4-(7-(1-methyl- 1H-pyrazol-4-yl)imidazo[1,2- c]pyrimidin-5-yl)-1H-pyrazol-1- yl)propan-1-ol | ES − APCI [M − 1] 390.2 |
| 73 | 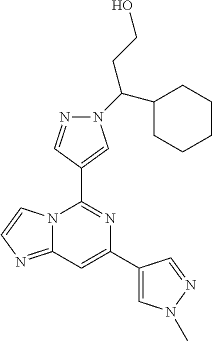 [SMILES: Cn1cc(-c2cc3nccn3c(-c3cnn(C(CCO)C4CCCCC4)c3)n2)cn1] | 3-cyclohexyl-3-(4-(7-(1-methyl- 1H-pyrazol-4-yl)imidazo[1,2- c]pyrimidin-5-yl)-1H-pyrazol-1- yl)propan-1-ol | ES − APCI [M − 1] 404.4 |
| 74 | 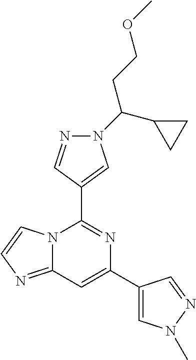 [SMILES: COCCC(C1CC1)n1cc(-c2nc(-c3cnn(C)c3)cc3nccn23)cn1] | 5-(1-(1-cyclopropyl-3- methoxypropyl)-1H-pyrazol-4-yl)- 7-(1-methyl-1H-pyrazol-4- yl)imidazo[1,2-c]pyrimidine | ES + APCI [M + 1] 378.2 |
| 75 | 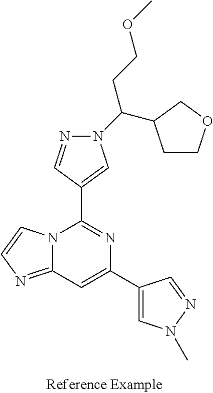 [SMILES: COCCC(C1CCOC1)n1cc(-c2nc(-c3cnn(C)c3)cc3nccn23)cn1] | 5-(1-(3-methoxy-1- (tetrahydrofuran-3-yl)propyl)-1H- pyrazol-4-yl)-7-(1-methyl-1H- pyrazol-4-yl)imidazo[1,2- c]pyrimidine | ES + APCI [M + 1] 408.2 |
| 76 | 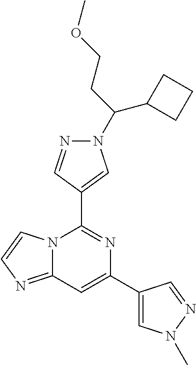 [SMILES: COCCC(C1CCC1)n1cc(-c2nc(-c3cnn(C)c3)cc3nccn23)cn1] | 5-(1-(1-cyclobutyl-3- methoxypropyl)-1H-pyrazol-4-yl)- 7-(1-methyl-1H-pyrazol-4- yl)imidazo[1,2-c]pyrimidine | ES + APCI [M + 1] 392.2 |
| 77 | 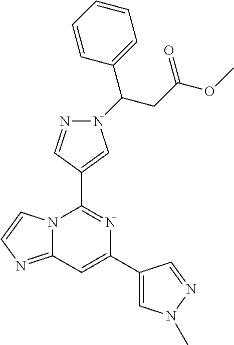 [SMILES: COC(=O)CC(c1ccccc1)n1cc(-c2nc(-c3cnn(C)c3)cc3nccn23)cn1] | methyl 3-(4-(7-(1-methyl-1H- pyrazol-4-yl)imidazo[1,2- c]pyrimidin-5-yl)-1H-pyrazol-1- yl)-3-phenylpropanoate | m/z ES + APCI [M + 1] 428.2 |
| 78 | 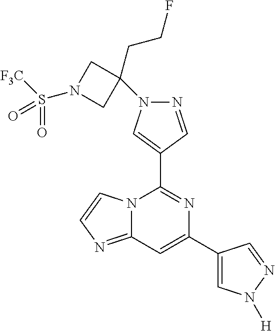 [SMILES: O=S(=O)(N1CC(CCF)(n2cc(-c3nc(-c4cn[nH]c4)cc4nccn34)cn2)C1)C(F)(F)F] | 5-(1-(3-(2-fluoroethyl)-1- ((trifluoromethyl)sulfonyl)azetidin- 3-yl)-1H-pyrazol-4-yl)-7-(1H- pyrazol-4-yl)imidazo[1,2- c]pyrimidine | ES + APCI [M + 1] 485.1 |
| 79 | 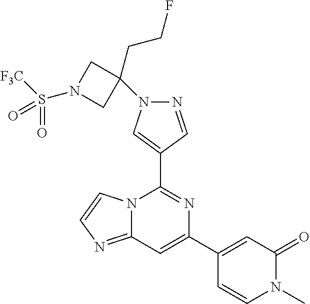 [SMILES: Cn1ccc(-c2cc3nccn3c(-c3cnn(C4(CCF)CN(S(=O)(=O)C(F)(F)F)C4)c3)n2)cc1=O] | 4-(5-(1-(3-(2-fluoroethyl)-1- ((trifluoromethyl)sulfonyl)azetidin- 3-yl)-1H-pyrazol-4- yl)imidazo[1,2-c]pyrimidin-7-yl)- 1-methylpyridin-2(1H)-one | ES + APCI [M + 1] 526.1 |
| 80 | 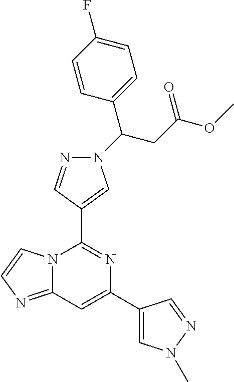 [SMILES: COC(=O)CC(c1ccc(F)cc1)n1cc(-c2nc(-c3cnn(C)c3)cc3nccn23)cn1] | methyl 3-(4-fluorophenyl)-3-(4-(7- (1-methyl-1H-pyrazol-4- yl)imidazo[1,2-c]pyrimidin-5-yl)- 1H-pyrazol-1-yl)propanoate | m/z ES + APCI [M + 1] 446.2 |
| 81 | 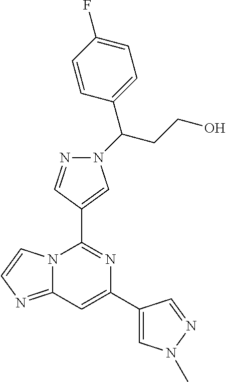 [SMILES: Cn1cc(-c2cc3nccn3c(-c3cnn(C(CCO)c4ccc(F)cc4)c3)n2)cn1] | 3-(4-fluorophenyl)-3-(4-(7-(1- methyl-1H-pyrazol-4- yl)imidazo[1,2-c]pyrimidin-5-yl)- 1H-pyrazol-1-yl)propan-1-ol | ES − APCI [M − 1] 416.1 |
| 82 | 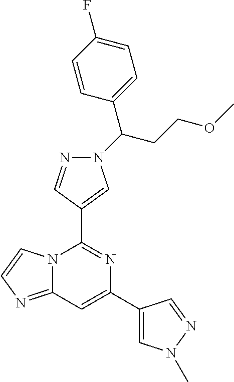 [SMILES: COCCC(c1ccc(F)cc1)n1cc(-c2nc(-c3cnn(C)c3)cc3nccn23)cn1] | 5-(1-(1-(4-fluorophenyl)-3- methoxypropyl)-1H-pyrazol-4-yl)- 7-(1-methyl-1H-pyrazol-4- yl)imidazo[1,2-c]pyrimidine | m/z ES + APCI [M + 1] 432.2 |
| 83 | 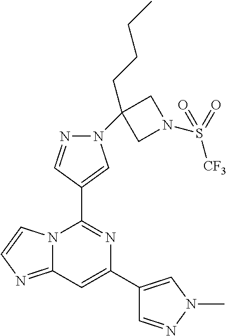 [SMILES: CCCCC1(n2cc(-c3nc(-c4cnn(C)c4)cc4nccn34)cn2)CN(S(=O)(=O)C(F)(F)F)C1] | 5-(1-(3-butyl-1- ((trifluoromethyl)sulfonyl)azetidin- 3-yl)-1H-pyrazol-4-yl)-7-(1- methyl-1H-pyrazol-4- yl)imidazo[1,2-c]pyrimidine | ES + APCI [M + 1] 509.1 |
| 84 | 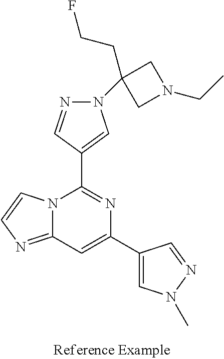 [SMILES: CCN1CC(CCF)(n2cc(-c3nc(-c4cnn(C)c4)cc4nccn34)cn2)C1] | 5-(1-(1-ethyl-3-(2- fluoroethyl)azetidin-3-yl)-1H- pyrazol-4-yl)-7-(1-methyl-1H- pyrazol-4-yl)imidazo[1,2- c]pyrimidine | m/z ES + APCI [M + 1] 395.2 |
| 85 | 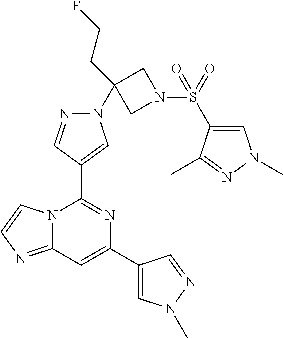 [SMILES: Cc1nn(C)cc1S(=O)(=O)N1CC(CCF)(n2cc(-c3nc(-c4cnn(C)c4)cc4nccn34)cn2)C1] | 5-(1-(1-((1,3-dimethyl-1H-pyrazol- 4-yl)sulfonyl)-3-(2- fluoroethyl)azetidin-3-yl)-1H- pyrazol-4-yl)-7-(1-methyl-1H- pyrazol-4-yl)imidazo[1,2- c]pyrimidine | ES + APCI [M + 1] 525.2 |
| 86 | 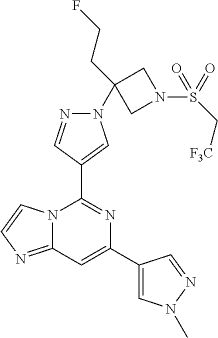 [SMILES: Cn1cc(-c2cc3nccn3c(-c3cnn(C4(CCF)CN(S(=O)(=O)CC(F)(F)F)C4)c3)n2)cn1] | 5-(1-(3-(2-fluoroethyl)-1-((2,2,2- trifluoroethyl)sulfonyl)azetidin-3- yl)-1H-pyrazol-4-yl)-7-(1-methyl- 1H-pyrazol-4-yl)imidazo[1,2- c]pyrimidine | ES + APCI [M + 1] 513.1 |
| 87 | 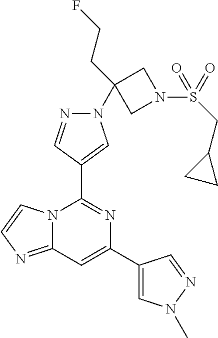 [SMILES: Cn1cc(-c2cc3nccn3c(-c3cnn(C4(CCF)CN(S(=O)(=O)CC5CC5)C4)c3)n2)cn1] | 5-(1-(1- ((cyclopropylmethyl)sulfonyl)-3- (2-fluoroethyl)azetidin-3-yl)-1H- pyrazol-4-yl)-7-(1-methyl-1H- pyrazol-4-yl)imidazo[1,2- c]pyrimidine | ES + APCI [M + 1] 485.1 |
| 88 | 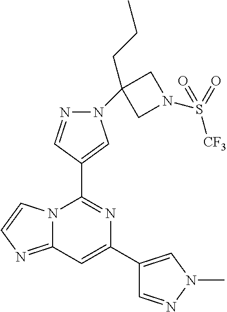 [SMILES: CCCC1(n2cc(-c3nc(-c4cnn(C)c4)cc4nccn34)cn2)CN(S(=O)(=O)C(F)(F)F)C1] | 7-(1-methyl-1H-pyrazol-4-yl)-5- (1-(3-propyl-1- ((trifluoromethyl)sulfonyl)azetidin- 3-yl)-1H-pyrazol-4- yl)imidazo[1,2-c]pyrimidine | m/z ES + APCI [M + 1] 495.6 |
| 89 | 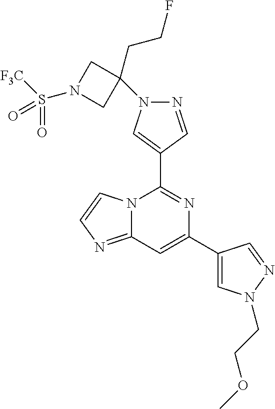 [SMILES: COCCn1cc(-c2cc3nccn3c(-c3cnn(C4(CCF)CN(S(=O)(=O)C(F)(F)F)C4)c3)n2)cn1] | 5-(1-(3-(2-fluoroethyl)-1- ((trifluoromethyl)sulfonyl)azetidin- 3-yl)-1H-pyrazol-4-yl)-7-(1-(2- methoxyethyl)-1H-pyrazol-4- yl)imidazo[1,2-c]pyrimidine | ES + APCI [M + 1] 543.1 |
| 90 | 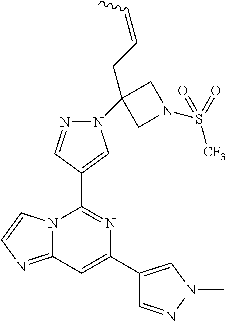 | 5-(1-(3-(but-2-en-1-yl)-1- ((trifluoromethyl)sulfonyl)azetidin- 3-yl)-1H-pyrazol-4-yl)-7-(1- methyl-1H-pyrazol-4- yl)imidazo[1,2-c]pyrimidine | ES + APCI [M + 1] 507.1 |
| 91 | 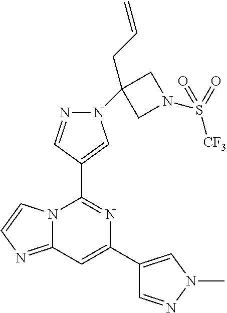 [SMILES: C=CCC1(n2cc(-c3nc(-c4cnn(C)c4)cc4nccn34)cn2)CN(S(=O)(=O)C(F)(F)F)C1] | 5-(1-(3-allyl-1- ((trifluoromethyl)sulfonyl)azetidin- 3-yl)-1H-pyrazol-4-yl)-7-(1- methyl-1H-pyrazol-4- yl)imidazo[1,2-c]pyrimidine | ES + APCI [M + 1] 493.1 |
| 92 | 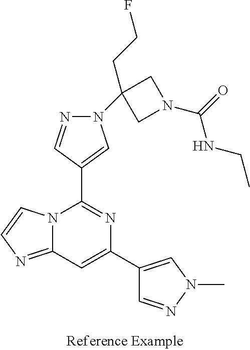 [SMILES: CCNC(=O)N1CC(CCF)(n2cc(-c3nc(-c4cnn(C)c4)cc4nccn34)cn2)C1] | N-ethyl-3-(2-fluoroethyl)-3-(4-(7- (1-methyl-1H-pyrazol-4- yl)imidazo[1,2-c]pyrimidin-5-yl)- 1H-pyrazol-1-yl)azetidine-1- carboxamide | m/z ES + APCI [M + 1] 438.1 |
| 93 | 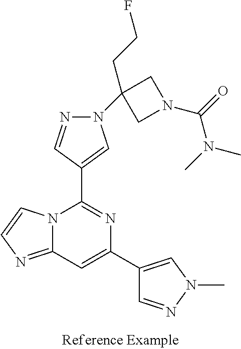 [SMILES: CN(C)C(=O)N1CC(CCF)(n2cc(-c3nc(-c4cnn(C)c4)cc4nccn34)cn2)C1] | 3-(2-fluoroethyl)-N,N-dimethyl-3- (4-(7-(1-methyl-1H-pyrazol-4- yl)imidazo[1,2-c]pyrimidin-5-yl)- 1H-pyrazol-1-yl)azetidine-1- carboxamide | m/z ES + APCI [M + 1] 438.1 |
| 94 | 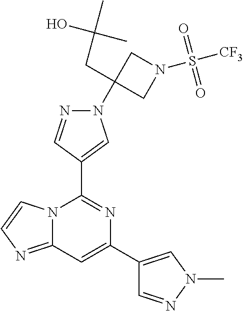 [SMILES: Cn1cc(-c2cc3nccn3c(-c3cnn(C4(CC(C)(C)O)CN(S(=O)(=O)C(F)(F)F)C4)c3)n2)cn1] | 2-methyl-1-(3-(4-(7-(1-methyl-1H- pyrazol-4-yl)imidazo[1,2- c]pyrimidin-5-yl)-1H-pyrazol-1- yl)-1- ((trifluoromethyl)sulfonyl)azetidin- 3-yl)propan-2-ol | m/z ES + APCI [M − 1] 523.1 |
| 95 | 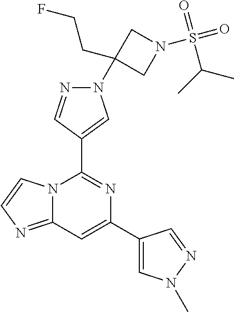 [SMILES: CC(C)S(=O)(=O)N1CC(CCF)(n2cc(-c3nc(-c4cnn(C)c4)cc4nccn34)cn2)C1] | 5-(1-(3-(2-fluoroethyl)-1- (isopropylsulfonyl)azetidin-3-yl)- 1H-pyrazol-4-yl)-7-(1-methyl-1H- pyrazol-4-yl)imidazo[1,2- c]pyrimidine | ES + APCI [M + 1] 473.1 |
| 96 | 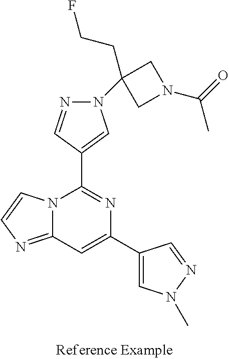 [SMILES: CC(=O)N1CC(CCF)(n2cc(-c3nc(-c4cnn(C)c4)cc4nccn34)cn2)C1] | 1-(3-(2-fluoroethyl)-3-(4-(7-(1- methyl-1H-pyrazol-4- yl)imidazo[1,2-c]pyrimidin-5-yl)- 1H-pyrazol-1-yl)azetidin-1- yl)ethanone | m/z ES + APCI [M + 1] 410.2 |
| 97 | 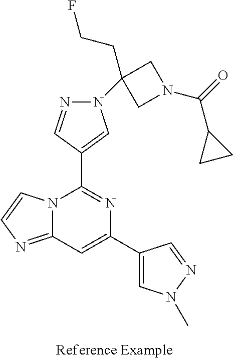 [SMILES: Cn1cc(-c2cc3nccn3c(-c3cnn(C4(CCF)CN(C(=O)C5CC5)C4)c3)n2)cn1] | cyclopropyl(3-(2-fluoroethyl)-3-(4- (7-(1-methyl-1H-pyrazol-4- yl)imidazo[1,2-c]pyrimidin-5-yl)- 1H-pyrazol-1-yl)azetidin-1- yl)methanone | m/z ES + APCI [M + 1] 435.2 |
| 98 | 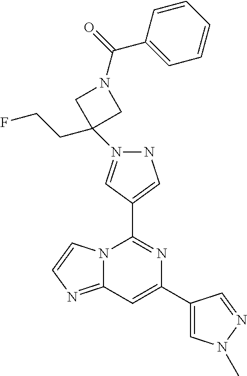 | (3-(2-fluoroethyl)-3-(4-(7-(1- methyl-1H-pyrazol-4- yl)imidazo[1,2-c]pyrimidin-5-yl)- 1H-pyrazol-1-yl)azetidin-1- yl)(phenyl)methanone | m/z ES + APCI [M + 1] 471.2 |
| 99 | 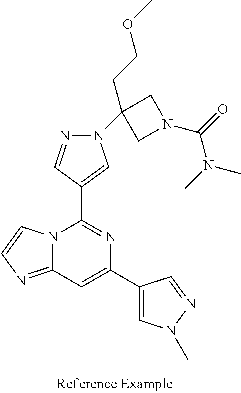 [SMILES: COCCC1(n2cc(-c3nc(-c4cnn(C)c4)cc4nccn34)cn2)CN(C(=O)N(C)C)C1] | 3-(2-methoxyethyl)-N,N-dimethyl- 3-(4-(7-(1-methyl-1H-pyrazol-4- yl)imidazo[1,2-c]pyrimidin-5-yl)- 1H-pyrazol-1-yl)azetidine-1- carboxamide | m/z ES + APCI [M + 1] 450.2 |
| 100 | 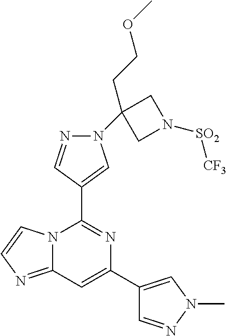 [SMILES: COCCC1(n2cc(-c3nc(-c4cnn(C)c4)cc4nccn34)cn2)CN(S(=O)(=O)C(F)(F)F)C1] | 5-(1-(3-(2-methoxyethyl)-1- ((trifluoromethyl)sulfonyl)azetidin- 3-yl)-1H-pyrazol-4-yl)-7-(1- methyl-1H-pyrazol-4- yl)imidazo[1,2-c]pyrimidine | ES + APCI [M + 1] 511.1 |
| 101 | 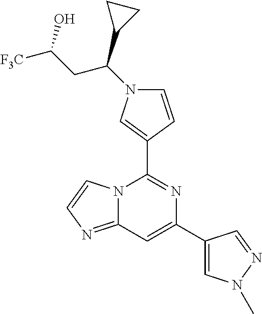 [SMILES: Cn1cc(-c2cc3nccn3c(-c3ccn([C@@H](C[C@@H](O)C(F)(F)F)C4CC4)c3)n2)cn1] | (2R,4S)-4-cyclopropyl-1,1,1- trifluoro-4-(3-(7-(1-methyl-1H- pyrazol-4-yl)imidazo[1,2- c]pyrimidin-5-yl)-1H-pyrrol-1- yl)butan-2-ol | ES − APCI [M − 1] 429.1 |
| 102 | 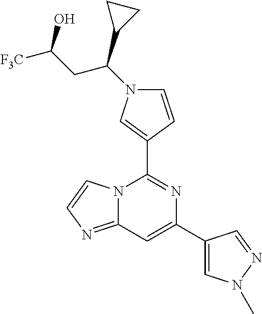 [SMILES: Cn1cc(-c2cc3nccn3c(-c3ccn([C@@H](C[C@H](O)C(F)(F)F)C4CC4)c3)n2)cn1] | (2S,4S)-4-cyclopropyl-1,1,1- trifluoro-4-(3-(7-(1-methyl-1H- pyrazol-4-yl)imidazo[1,2- c]pyrimidin-5-yl)-1H-pyrrol-1- yl)butan-2-ol | ES − APCI [M − 1] 429.1 |

1. A compound of the general Formula I 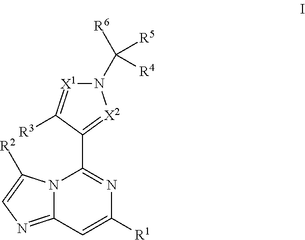
and stereoisomers and pharmaceutically acceptable salts and solvates thereof, wherein:
X¹ is N or CR^{3b};
X² is N or CR^{3a};
R¹ is hetAr¹, hetAr², hetAr³, Ar¹, Ar², (3-6C)cycloalkyl or N-(1-3C alkyl)pyridinonyl;
hetAr¹ is a 5 membered heteroaryl ring having 1-3 ring heteroatoms independently selected from N, O and S and optionally substituted with one or more substituents independently selected from halogen, (1-6C)alkyl, fluoro(1-6C)alkyl, difluoro(1-6C)alkyl, trifluoro(1-6C)alkyl, (1-4C alkoxy)(1-6C)alkyl, trimethylsilyl(1-4C alkoxy)(1-6C)alkyl, (3-6C)cycloalkyl, a 4-6 membered oxacyclic ring, hetCyc^{a}(1-2C)alkyl, hetAr^{a}(1-2C)alkyl and (1-4C alkylsulfonyl)(1-6C alkyl);
hetCyc^{a} is a 6 membered heterocycle having 1-2 ring heteroatoms independently selected from N and O and is optionally substituted with (1-6C)alkyl;
hetAr^{a} is a 6 membered heteroaryl having 1-2 ring nitrogen atoms;
hetAr² is a 9-membered bicyclic partially unsaturated or fully unsaturated heterocyclic ring having 3 ring nitrogen atoms and optionally substituted with one or more substituents independently selected from (1-6C)alkyl;
hetAr³ is a 6 membered heteroaryl having 1-2 ring nitrogen atoms and optionally substituted with one or more substituents independently selected from (1-6C)alkyl, hetCyc^{b} and (1-6C)alkoxy;
hetCyc^{b} is a 6-membered heterocycle having 1-2 ring nitrogen atoms and optionally substituted with one or more substituents independently selected from (1-6C)alkyl;
Ar¹ is phenyl substituted with a substituent selected from halogen, hetCyc^{c}, hetCyc^{d}, hetAr^{b}, trifluoro(1-6C)alkyl and (1-6C)alkoxy;
hetCyc^{c} is a 6 membered heterocycle having 1-2 ring heteroatoms independently selected from N and O and optionally substituted with one or more substituents independently selected from (1-6C)alkyl;
hetCyc^{d} is an 8-membered bridged heterocyclic ring having 1-2 ring heteroatoms independently selected from N and O;
hetAr^{b} is a 5-membered heteroaryl ring having 1-2 ring nitrogen atoms and optionally substituted with one or more substituents independently selected from (1-6C)alkyl;
Ar² is a benzo ring fused to a 5-6 membered azacyclic ring and is optionally substituted with one or more substituents independently selected from (1-6C)alkyl;
R² is hydrogen, halogen, (1-4C)alkyl, CF₃, CN, or (3-4C)cycloalkyl;
R³, R^{3a} and R^{3b} are independently hydrogen, (1-6C)alkyl, CF₃, F, Cl, CN or (3-6C)cycloalkyl;
R⁴ is hydrogen, and
R⁵ is hydrogen, (3-6C)cycloalkyl (optionally substituted by one or more halogens), (3-6C)cycloalkylCH₂— (optionally substituted by one or more halogens), (1-6C)alkyl, a 4-6 membered heterocycle having 1-2 ring heteroatoms independently selected from N, O and S, or phenyl optionally substituted with one or more halogens,
or R⁴ and R⁵ together with the carbon atom to which they are attached form a 4- or 5-membered azacyclic ring substituted with a substituent selected from fluoro(1-6C)alkyl, difluoro(1-6C)alkyl, trifluoro(1-6C)alkyl, (1-6Calkyl)C(═O)O—, —SO₂R^{c}, (1-6C)alkyl, (1-6Calkyl)C(═O)—, phenylC(═O)—, cyclopropyl-C(═O)—, (1-6C alkyl)NHC(═O)—, di(1-6C alkyl)NC(═O)—, or cyano(1-6Calkyl),
or R⁴ and R⁵ together with the carbon atom to which they are attached form a 3-6-membered carbocyclic ring optionally substituted with one or more substitutents independently selected from methyl and halogen;
R^{c} is H, fluoro(1-3C)alkyl, difluoro(1-3C)alkyl trifluoro(1-3C)alkyl, (3-6C)cycloalkyl, cyclopropylamino, cyclopropylmethyl, (1-6C)alkyl, or a 5-membered heteroaryl having 1-2 ring heteroatoms independently selected from N, O and S, wherein said 5-membered heteroaryl is optionally substituted with one or more substituents independently selected from (1-6C)alkyl; and
R⁶ is H, (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, hydroxy(1-6C)alkyl, (1-3C alkoxy)(1-6C)alkyl, (1-3C alkylsufanyl)(1-3C)alkyl, (1-3C alkyl)OC(═O)(1-3C)alkyl, carboxy(1-6C)alkyl, fluoro(2-6C)alkenyl, difluoro(2-6C)alkenyl or (1-6C)alkylC(═O)CH₂—.

2. (canceled)

3. A compound according to claim 1, wherein R¹ is hetAr¹.

4. A compound according to claim 3, wherein hetAr¹ is pyrazolyl, thiazolyl, oxazolyl, thiadiazolyl, imidazolyl, pyrrolyl or thiophenyl optionally substituted with one or more substituents independently selected from halogen, (1-6C)alkyl, fluoro(1-6C)alkyl, difluoro(1-6C)alkyl, trifluoro(1-6C)alkyl, (1-4C alkoxy)(1-6C)alkyl, trimethylsilyl(1-4C alkoxy)(1-6C)alkyl, (3-6C)cycloalkyl, a 4-6 membered oxacyclic ring, hetCyc^{a}(1-2C)alkyl, hetAr^{a}(1-2C)alkyl and (1-4C alkylsulfonyl)(1-6C alkyl).

5. (canceled)

6. A compound according to claim 4, wherein hetAr¹ is pyrazol-4-yl optionally substituted with a substituent selected from (1-6C)alkyl.

7-9. (canceled)

10. A compound according to claim 1, wherein R⁴ is hydrogen and R⁵ is hydrogen, (3-6C)cycloalkyl or (3-6C)cycloalkylCH₂—.

11. (canceled)

12. A compound according to claim 1, wherein R⁴ and R⁵ together with the carbon atom to which they are attached form a 4-membered azacyclic ring substituted with a substituent selected from fluoro(1-6C)alkyl, difluoro(1-6C)alkyl, trifluoro(1-6C)alkyl, (1-6Calkyl)C(═O)O— and —SO₂R^{c}.

13. A compound according to claim 12, wherein R⁴ and R⁵ together with the carbon atom to which they are attached form a 4-membered azacyclic ring substituted with a substituent selected from fluoro(1-6C)alkyl, difluoro(1-6C)alkyl and trifluoro(1-6C)alkyl.

14. (canceled)

15. A compound according to claim 12, wherein R⁴ and R⁵ together with the carbon atom to which they are attached form a 4-membered azacyclic ring substituted with —SO₂R^{c}.

16. A compound according to claim 15, wherein R⁴ and R⁵ together with the carbon atom to which they are attached form a 4-membered azacyclic ring substituted with —SO₂CH₃, —SO₂CH₂CH₃, —SO₂CH₂CH₂CH₃, —SO₂CH(CH₃)₂, —SO₂CH₂CH₂CF₃, —SO₂CF₃, —SO₂CF₂CF₃, SO₂CF₂H or —SO₂-cyclopropyl.

17-19. (canceled)

20. A compound according to claim 1, where R⁶ is (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl or (3-6C cycloalkyl)(1-3C)alkyl.

21. (canceled)

22. A compound according to claim 20, where R⁶ is fluoro(1-6C)alkyl, difluoro(1-6C)alkyl or trifluoro(1-6C)alkyl.

23. (canceled)

24. A compound according to claim 1, where R² is hydrogen.

25. A compound according to claim 1, where R³ and R^{3a} are independently selected from hydrogen, (1-6C alkyl), CF₃, F and Cl.

26. A compound according to claim 25, where R³ and R^{3a} are hydrogen.

27. A compound according to claim 1, wherein X¹ is N and X² is CR^{3a}.

28. A compound according to claim 1, wherein X¹ is CR^{3b} and X² is CR^{3a}.

29. A compound selected from any one of Examples 1-74, 76-83, 85-91, 94, 95, 98 and 100-102.

30. A pharmaceutical composition, which comprises a compound of Formula I as defined in claim 1 or a pharmaceutically acceptable salt or solvate thereof, and a pharmaceutically acceptable diluent or carrier.

31. A method for treating an autoimmune disease or inflammatory disease in a mammal, which comprises administering to said mammal a therapeutically effective amount of a compound of Formula I as defined in claim 1 or a pharmaceutically acceptable salt or solvate thereof.

32. A method for treating organ, tissue or cell transplant rejection in a mammal, which comprises administering to said mammal a therapeutically effective amount of a compound of Formula I as defined in claim 1 or a pharmaceutically acceptable salt or solvate thereof.

33. A method for treating a malignancy in a mammal, which comprises administering to said mammal a therapeutically effective amount of a compound of Formula I as defined in claim 1 or a pharmaceutically acceptable salt or solvate thereof.

34. (canceled)

35. A process for the preparation of a compound of claim 1 or a pharmaceutically acceptable salt thereof, which comprises:
(a) for a compound of Formula I where R⁴ is hydrogen; R⁵ is hydrogen, (3-6C)cycloalkyl (optionally substituted by one or more halogens) or (3-6C)cycloalkylCH₂-(optionally substituted by one or more halogens); and R⁶ is (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, and R¹, R², R³, X¹ and X² are as defined for Formula I, reacting a corresponding compound of formula II 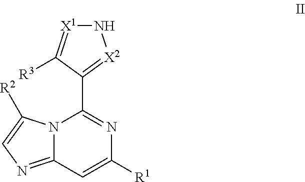
with a corresponding compound having the formula 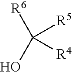
where R⁴ is hydrogen; R⁵ is hydrogen, (3-6C)cycloalkyl (optionally substituted by one or more halogens) or (3-6C)cycloalkylCH₂— (optionally substituted by one or more halogens); and R⁶ is (1-6C)alkyl, (2-6C)alkenyl, (2-6C)alkynyl, (3-6C)cycloalkyl, fluoro(1-6C)alkyl, difluoro(1-6C)alkyl, trifluoro(1-6C)alkyl, (3-6C cycloalkyl)(1-3C)alkyl, in the presence of triphenylphosphine and a coupling agent; or
(b) for a compound of Formula I where R⁶ is HOCH₂CH₂—; and R¹, R², R³, R⁴, R⁵, X¹ and X² are as defined for Formula I, treating a corresponding compound having the formula 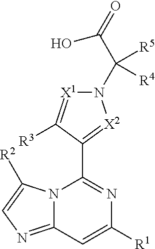
with a reducing agent; or
(c) for a compound of Formula I where R⁶ is methoxy(1-6C)alkyl; and R¹, R², R³, R⁴, R⁵, X¹ and X² are as defined for Formula I, treating a corresponding compound where R⁶ is hydroxy(1-6C)alkyl with methyl iodide in the presence of a base; or
(d) for a compound of Formula I where R⁶ is HOCH₂—; R⁵ is (3-6C)cycloalkyl; R⁴ is hydrogen; and R¹, R², R³, X¹ and X² are as defined for Formula I, reacting a compound of Formula II 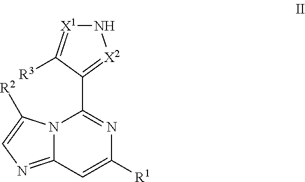
with a compound having the formula: 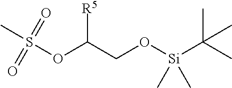
in the presence of a base; or
(e) for a compound Formula I where R⁶ is (1-3Calkyl)OC(═O)CH₂—; R⁵ is (3-6C)cycloalkyl; R⁴ is hydrogen; and R¹, R², R³, X¹ and X² are as defined for Formula I, reacting a compound of formula II 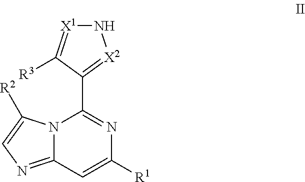
with a compound having the formula 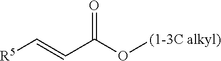
in the presence of 2,8,9-triisobutyl-2,5,8,9-tetraaza-1-phosphabicyclo[3.3.3]undecane; or
(f) for a compound of Formula I where R⁶ is fluoro(1-6C)alkyl; and R¹, R², R³, R⁴, R⁵, X¹ and X² are as defined for Formula I, reacting a corresponding compound of Formula I′ 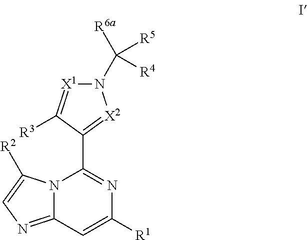
where R^{6a} is CH₃SO₃(1-6C)alkyl, and R¹, R², R³, R⁴, R⁵, X¹ and X² are as defined for Formula I, with tetrabutylammonium fluoride; or
(g) for a compound of Formula I wherein R⁴ and R⁵ form a 4-membered azacyclic ring substituted with fluoro(1-6C)alkyl, difluoro(1-6C)alkyl or trifluoro(1-6C)alkyl, and R¹, R², R³, R⁶, X¹ and X² are as defined for Formula I, coupling a corresponding compound having the formula III 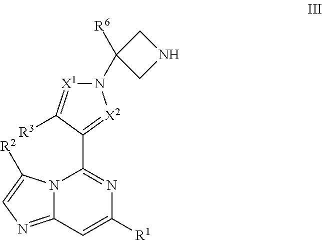
with a corresponding compound having the formula L³-R¹⁰, where L³ is a leaving group or atom and R¹⁰ is fluoro(1-6C)alkyl, difluoro(1-6C)alkyl or trifluoro(1-6C)alkyl, in the presence of a base; or
(h) for a compound of Formula I wherein R⁴ and R⁵ form a 4-membered azacyclic ring substituted with SO₂CF₃, and R¹, R², R³, R⁶, X¹ and X² are as defined for Formula I, reacting a corresponding compound having the formula III 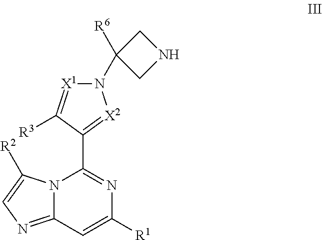
with trifluoromethanesulfonic anhydride in the presence of a base; or
(i) for a compound of Formula I wherein R⁴ and R⁵ form a 4-membered azacyclic ring substituted with SO₂R^{c}, wherein R^{c}, R¹, R², R³, R⁶, X¹ and X² are as defined for Formula I, coupling a corresponding compound having the formula III 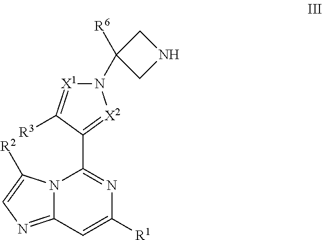
with a corresponding compound having the formula Cl—SO₂R^{c} in the presence of a base; or
(j) for a compound of Formula I wherein R² is Cl, and R¹, R³, R⁴, R⁵, R⁶, X¹ and X² are as defined for Formula I, reacting a corresponding compound of Formula I″ 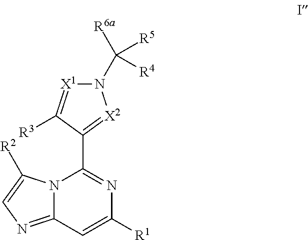
wherein R² is hydrogen, and R¹, R³, R⁴, R⁵, R⁶, X¹ and X² are as defined for Formula I, with 1-chloropyrrolidine-2,5-dione; or
(k) for a compound of Formula I wherein R² is CN, and R¹, R³, R⁴, R⁵, R⁶, X¹ and X² are as defined for Formula I, reacting a corresponding compound of Formula I″ 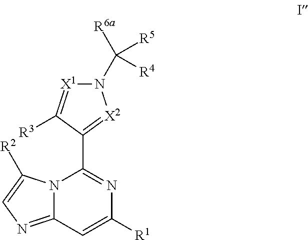
wherein R² is hydrogen, and R¹, R³, R⁴, R⁵, R⁶, X¹ and X² are as defined for Formula I, with 1-iodopyrrolidinine-2,5-dione followed by treatment of the resulting 3-iodo-substituted derivative of I′ with CuCN; or
(l) for a compound of Formula I wherein R² is F, and R¹, R³, R⁴, R⁵, R⁶, X¹ and X² are as defined for Formula I, reacting a corresponding compound of Formula I″ 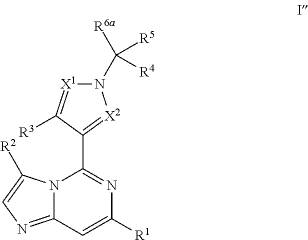
wherein R² is hydrogen, and R¹, R³, R⁴, R⁵, R⁶, X¹ and X² are as defined for Formula I, with an electrophilic fluorinating agent; or
(m) for a compound of Formula I wherein R² is F, and R¹, R³, R⁴, R⁵, R⁶, X¹ and X² are as defined for Formula I, reacting a corresponding compound of Formula I″′ 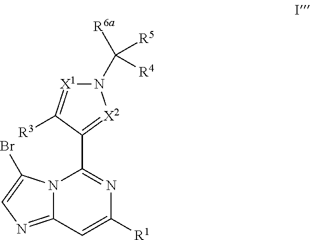
with an alkyl lithium or alkyl magnesium halide reagent, followed by treatment with an electrophilic fluorinating agent; and
optionally removing any protecting groups and optionally preparing a pharmaceutically acceptable salt thereof.
